SEQUENCE LISTING (11 sequences)

SEQ ID NO: 1 - DNA, 22 bp, Artificial Sequence
  synthetic oligo
ttcctgtcctccatgatcaaaa

SEQ ID NO: 2 - DNA, 21 bp, Artificial Sequence
  synthetic oligo
catccacctctgttgggttca

SEQ ID NO: 3 - DNA, 19 bp, Artificial Sequence
  synthetic oligo
cacacccgccaccagttcg

SEQ ID NO: 4 - DNA, 19 bp, Artificial Sequence
  synthetic oligo
caccatcacaccctggtgc

SEQ ID NO: 5 - DNA, 20 bp, Artificial Sequence
  synthetic oligo
acccgccagtctcctacatc

SEQ ID NO: 6 - DNA, 20 bp, Artificial Sequence
  synthetic oligo
gcatctaggcgcagggattg

SEQ ID NO: 7 - DNA, 20 bp, Artificial Sequence
  synthetic oligo
caggccaaggtctacaatcg

SEQ ID NO: 8 - DNA, 20 bp, Artificial Sequence
  synthetic oligo
ttgatcccacaggcattctt

SEQ ID NO: 9 - DNA, 20 bp, Artificial Sequence
  synthetic oligo
attgtcattcccaatgatgg

SEQ ID NO: 10 - DNA, 22 bp, Artificial Sequence
  synthetic oligo
catagttctcatacagatggac

SEQ ID NO: 11 - RNA, 32 nt, Artificial Sequence
  synthetic oligo
ucaaacaguccucgcaugccuauagugagucg

(19) United States
(12) Patent Application Publication
(10) Pub. No.: US 2020/0108091 A1
(43) Pub. Date: Apr. 9, 2020

(54) GUT-PROTECTIVE EFFECT OF RIG-1/MAVS AND STING ACTIVATION

(71) Applicants: MEMORIAL SLOAN-KETTERING CANCER CENTER, New York, NY (US); KLINIKUM RECHTS DER ISAR DER TECHNISCHEN UNIVERSITÄT MÜNCHEN, München (DE)

(72) Inventors: Marcel VAN DEN BRINK, New York, NY (US); Hendrik POECK, München (DE)

(73) Assignees: MEMORIAL SLOAN-KETTERING CANCER CENTER, New York, NY (US); KLINIKUM RECHTS DER ISAR DER TECHNISCHEN UNIVERSITÄT MÜNCHEN, München (DE)

(21) Appl. No.: 16/605,883

(22) PCT Filed: Apr. 17, 2018
(86) PCT No.: PCT/US2018/027961

§ 371 Date: Oct. 17, 2019

Related U.S. Application Data
(60) Provisional application No. 62/589,845, filed on Nov. 22, 2017.
(60) Provisional application No. 62/486,213, filed on Apr. 17, 2017.

(51) Int. Cl. A61K 31/7105; A61K 31/711; A61P 1/00; A61P 37/06

(52) U.S. Cl. CPC A61K 31/7105; A61P 37/06; A61P 1/00; A61K 31/711

(57) ABSTRACT

Disclosed herein are methods for inhibiting irradiation- and chemo-induced impact on intestinal barrier function and graft versus host disease following allogeneic hematopoietic stem cell transplantation by targeting the RIG-I or STING signaling pathways.

### CROSS-REFERENCE TO RELATED APPLICATIONS

This application claims priority to U.S. provisional application No. 62/486,213 filed Apr. 17, 2017 and U.S. provisional application No. 62/589,845 filed Nov. 22, 2017; the contents of both are hereby incorporated by reference in their entirety into the present disclosure.

### STATEMENT OF RIGHTS UNDER FEDERALLY-SPONSORED RESEARCH

This invention was made with government support under HL069929, AI100288, AI080455, AI101406, CA023766 and CA008748 awarded by the National Institutes of Health. The government has certain rights in the invention.

### SEQUENCE LISTING

The instant application contains a Sequence Listing, created on Jul. 24, 2017; the file, in ASCII format, is designated 3314087AWO_SequenceListing_ST25.txt and is 2.26 KB in size. The file is hereby incorporated by reference in its entirety into the instant application.

### TECHNICAL FIELD

The present disclosure relates generally to activation of retinoic acid-inducible gene 1 (RIG-I) or STING signaling pathways, which guards against irradiation- and chemo-induced impact on intestinal barrier function and more particularly to the ability of RIG-I agonists to reduce/prevent graft versus host disease (GVHD).

### BACKGROUND OF THE DISCLOSURE

Allogeneic hematopoietic stem cell transplantation is a treatment of choice for a range of malignant and nonmalignant disorders. Pre-transplant conditioning requires ablation of the patient's own hematopoietic cells either by total body irradiation (TBI) or chemotherapeutic agent followed by introduction of the allogeneic HSCs into the patient. One complication of the conditioning is graft versus host disease (GVHD) in which the transplanted stem cells become T lymphocytes and start attacking the host's own cells.

During conditioning, the drug treatment and irradiation damage the epithelial cells that form part of the intestinal mucosal. Loss of the intestinal epithelial layer is believed to be the trigger for GVHD.

In an effort to identify a mechanism for protecting the intestinal epithelium, the inventors explored the role of two proteins in the body known for their role in fighting bacteria and viruses: RIG-I and STING. RIG-I belongs to the pattern recognition family of cytoplasmic RIG-I-like receptors. Its primary function is to detect double-stranded 5′-triphosphate RNA (3pRNA) during viral or bacterial infection (1-3). In contrast, the cytosolic DNA receptor cyclic guanosine monophosphate-adenosine monophosphate (cGAMP) synthase (cGAS) and its adapter protein STING (STimulator of INterferon Genes, TMEM173), recognize DNA in various contexts e.g. microbial DNA or nuclear DNA released into the cytosol by necrotic cells (4).

Upon binding of ligand, RIG-I recruits the adaptor mitochondrial antiviral-signaling protein (MAVS) to induce pro-inflammatory cytokines, type-I interferons (IFN-I) and inflammasome activation (1, 5-8), orchestrating a diverse innate and adaptive immune response. cGAS binds to double-stranded (ds) DNA and catalyzes the formation of cyclic dinucleotides. The latter can form cyclic GAMP (cGMP-AMP=2′3 cGAMP) which activates STING to trigger innate immune gene transcription and IFN-I production (4). Whereas the role of IFN-I in initiating host defense against pathogens is well established, recent work highlights the regenerative function of this cytokine family, particularly at epithelial surfaces. IFN-I produced by plasmacytoid dendritic cells (pDC) promotes skin repair upon mechanical barrier disruption (9) and increases intestinal epithelial turnover and repair of chemically damaged tissue. The effects of IFN-I on gut epithelial turnover have been attributed to both macrophage-dependent mechanisms (10) and Toll-like receptor (TLR) stimulation of pDC (11). However, the role of cytosolic nucleic acid sensors in this context is poorly understood.

Similarly, the involvement of IFN-I in the repair of acute tissue damage by genotoxic insults has not been addressed. Unlike chemical injury of intestinal mucosa, irradiation-induced or chemotherapy-induced intestinal barrier dysfunction is a problem clinically.

Mucosal barriers like the intestinal epithelial cell (IEC) layer protect sterile microenvironments from physical, chemical and microbial challenge. Epithelial integrity depends on constant and inducible IEC renewal by pluripotent intestinal stem cells (ISCs) which reside in the stem cell niche at the base of each intestinal crypt (12). Genotoxic stress by total body irradiation (TBI) or chemotherapy affects ISC and results in damage to the intestinal epithelium, ultimately causing translocation of microbes to sterile compartments and subsequent immune activation (13).

During allogeneic hematopoietic stem cell transplantation (allo-HSCT), alteration of intestinal barrier function by chemotherapy or TBI administered pre-transplant has detrimental consequences: “misplaced” bacterial components together with endogenous danger signals released during epithelial cell death are sensed by pattern recognition receptors on antigen-presenting cells, which then produce pro-inflammatory cytokines and prime donor-derived T cells (13). These alloreactive T cells attack and destroy host tissues primarily the gastrointestinal (GI) tract, liver and skin, causing morbidity and mortality in a process called acute graft-versus-host disease (GVHD). GVHD is the leading complication after allo-HSCT and occurs in as many as 50% of transplant recipients.

Thus, investigating molecular mechanisms that promote intestinal epithelial integrity and repair during tissue injury is fundamental to the development of new approaches to prevent treatment-associated inflammation and GVHD. The RIG-I-MAVS and STING signaling pathways are important regulators of IFN-I production, and IFN-I can initiate epithelial repair. Thus, we hypothesized that activation of these pathways during pre-transplant bone marrow ablative therapy in mice may protect epithelial integrity and could be exploited to promote intestinal barrier function and prevent GVHD.

### SUMMARY OF THE DISCLOSURE

The present disclosure shows that properly timed therapeutic activation of RIG-I or cGAS/STING, for example by administration of a RIG-I agonist such as 3pRNA reduces gut epithelial barrier dysfunction, promotes epithelial integrity and prevents thymic damage during acute tissue damage caused by chemotherapy or TBI, providing a mechanism to prevent the development of GVHD.

In one aspect, therefore, the disclosure relates to a method for attenuating or inhibiting treatment-associated inflammation and GVHD comprising administering to a subject in need thereof a therapeutically effective amount of a RIG-I agonist, a STING agonist or a combination thereof.

In another aspect, the disclosure relates to a method for attenuating or inhibiting acute intestinal injury during allogeneic hematopoietic stem cell transplantation (HSCT or allo-HSCT) comprising administering to a subject in need thereof a therapeutically effective amount of a RIG-I agonist, a STING agonist or a combination thereof. In one embodiment the agonist is 3pRNA or double-stranded DNA including interferon stimulatory DNA (ISD). In some embodiments, administration of agonist occurs prior to allo-HSCT, e.g., agonist may be administered from 2-3 days prior to transplantation up until transplantation.

In yet another aspect, the disclosure relates to a method for inhibiting GVHD following allo-HSCT comprising administering to a subject in need thereof a therapeutically effective amount of a RIG-I agonist, a STING agonist or a combination thereof.

In yet another related aspect, the disclosure relates to a method to promote growth of intestinal organoids in vitro comprising contacting said intestinal organoids with a RIG-I agonist.

### BRIEF DESCRIPTION OF THE DRAWINGS

FIGS. 1A-1G show that endogenous RIG-I/MAVS signaling reduces intestinal tissue damage in mice. (A) Representative images of tissue damage in H&E stained small intestine biopsies from mice after 11Gy of a TBI conditioning regimen: white asterisk, villus blunting, black arrowhead, crypt apoptosis; black arrow, granulocyte infiltration. (B) Histopathological score from panel A; pooled data from 2 independent experiments. (C) The number of leukocytes infiltrating the mouse gut lamina propria after TBI (11Gy) analyzed by flow cytometry. Pooled data from 4 independent experiments. (D) Survival and (E) weight loss in mouse recipients after allo-HSCT with 5×10⁶ bone marrow (BM)−/+2×10⁶ T cells (donor BALB/c wild-type (WT), recipient C57BL/6 MAVS^{+/+} or MAVS^{-/-}). Pooled data of 4 independent experiments. (F) FITC-dextran concentrations in plasma after allo-HSCT as in D) and E). Pooled data of 2 independent experiments. (G) Survival of Rig-I^{-/+} and Rig-I^{-/-} mouse recipients after allo-HSCT with 5×10⁶ bone marrow (BM) and 1×10⁶ T cells (donor C57BL/6 wild-type (WT); recipient 129/sv Rig-I^{-/+} or Rig-I^{-/-}). Animal numbers per group (n) are depicted in the figure panels. Survival was analyzed using the Log-rank test. All other experiments were analyzed using two-tailed unpaired t test. Significance was set at p values <0.05, p<0.01 and p<0.001 and was then indicated with asterisks (*, ** and ***). Data are presented as mean±S.E.M. n.s., not significant.

FIGS. 2A-2D show that MAVS signaling in non-hematopoietic cells maintains intestinal barrier function. (A) Average relative abundance of bacterial genera in the intestinal microbiota of cohoused Mavs^{+/+} (n=5) and Mavs^{-/-} (n=5) littermates. One representative of 2 independent experiments. (B) Survival of C57BL/6 bone marrow (BM) chimeric mice which were Mavs-deficient either in the hematopoietic or in the non-hematopoietic compartment, analyzed after a second allo-HSCT with bone marrow and T cells from B10.BR wild-type (WT) donors. (C) Histopathological examination of small intestine biopsies and GVHD scores of C57BL/6 bone marrow chimeric mice which were Mavs-deficient either in the hematopoietic or in the non-hematopoietic compartment, analyzed after a second allo-HSCT with bone marrow (BM) and T cells from Balb/c wild-type (WT) donors. Pooled data from 2 independent experiments. (D) qPCR of ltgb6 and Reglllγ expression in the small intestine after allo-HSCT with bone marrow (BM) and T cells from BALB/c wild-type (WT) mouse donors into C57BL/6 mouse recipients. Pooled data of 5 independent experiments Animal numbers per group (n) are depicted above panels. Survival was analyzed using the Log-rank test. Other experiments were analyzed using ordinary one-way Anova for multiple comparisons or two-tailed unpaired t test. Significance was set at p values <0.05, p<0.01 and p<0.001 and was then indicated with asterisks (*, ** and ***). Data are presented as means±S.E.M.

FIGS. 3A-3K show that RIG-I/MAVS pathway activation protects from intestinal tissue damage after a TBI conditioning regimen. (A) Survival and (B) weight loss after allo-HSCT involving transplant of 5×10⁶ bone marrow (BM)−/+1×10⁶ T cells from C57BL/6 wild-type (WT) donor mice into BALB/c wild-type recipients, with or without treatment with 3pRNA on day −1. Pooled data of 4 independent experiments. (C) Histopathological score of small intestine tissue after allo-HSCT from C57BL/6 wild-type (WT) donor mice to BALB/c wild-type mouse recipients, with or without treatment with 3pRNA on day −1. Pooled data from 2 independent experiments. (D) Weight loss in mouse recipients from panel C after allo-HSCT in the presence or absence of 3pRNA treatment on day 0 or +1 (d0 or d+1). Pooled data from 3 independent experiments. (E) FITC-dextran concentrations in plasma after allo-HSCT from C57BL/6 wild-type (WT) donor mice to BALB/c wild-type recipient mice in the presence or absence of 3pRNA treatment on d −1. (F) qPCR of Reglllγ expression in mouse gut epithelial cells after allo-HSCT from C57BL/6 wild-type (WT) donor mice into BALB/c wild-type recipient mice in the presence or absence of 3pRNA treatment on d −1. Pooled data from 3 independent experiments. (G) Bacterial colony forming units (CFUs) in sera from mouse recipients after allo-HSCT as in (F). Pooled data from 3 independent experiments. (H) Leukocytes in the small intestine lamina propria of BALB/c mice after TBI (9Gy) analyzed by flow cytometry. Pooled data from 2 independent experiments. (I) Leukocytes in the small intestine lamina propria of C57BL/6 mice after treatment with chemotherapy (doxorubicin) analyzed by flow cytometry. Pooled data from 3 independent experiments. (J) Weight loss of C57BL/6 mice that received doxorubicin (20 mg/kg). Pooled data from 6 independent experiments. (K) FITC-dextran concentrations in plasma from C57BL/6 mice after doxorubicin treatment (20 mg/kg). One representative experiment of 4 independent experiments is shown. Animal numbers per group (n) are depicted above figure panels. 3pRNA treatment was always performed on day −1 (d−1) in indicated groups except for (D). All experiments were analyzed using one-tailed (G) or two-tailed unpaired t test or ordinary one-way Anova for multiple comparisons. Survival was analyzed using the Log-rank test. Significance was set at p values <0.05, p<0.01 and p<0.001 and was then indicated with asterisks (*, ** and ***). Data are presented as mean±S.E.M.

FIGS. 4A-4G show that RIG-I-induced type I IFN signaling mediates intestinal tissue protection. (A) Survival and percent of initial weight after allo-HSCT: 5×10⁶ bone marrow plus 1×10⁶ T cells of C57BL/6 wild-type (WT) mice transplanted into BALB/c recipient mice with or without treatment with 3pRNA on day −1 (d−1) before allo-HSCT, IFNaR1 blocking antibody (α-IFNAR1) or IgG1 Isotype control (IC) on day −2 (d−2) before allo-HSCT. Pooled data from 3 independent experiments. (B) Weight loss after allo-HSCT: 5×10⁶ bone marrow (BM) plus 1×10⁶ T cells of C57BL/6 wild-type (WT) mice transplanted into BALB/c recipient mice with or without treatment with 3pRNA on day −1 (d−1) before allo-HSCT, IFNaR1 blocking antibody (α-IFNAR1) 48 hours before allo-HSCT (−48 h before TX), at the time of allo-HSCT (0 h before TX) or 24 hours after allo-HSCT (24 h after TX). (C) FITC-dextran concentrations in plasma in BALB/c mouse recipients after allo-HSCT, with or without treatment with 3pRNA on day −1 (d−1) before allo-HSCT, IFNaR1 blocking antibody (α-IFNAR1) or IgG1 Isotype control (IC) on day −2 (d−2) before allo-HSCT. Pooled data of 3 independent experiments. (D, E) qPCR of Reglllγ and ltgb6 expression in small intestine biopsies from BALB/c recipient mice after allo-HSCT, with or without treatment with 3pRNA on day −1 (d−1) before allo-HSCT, IFNaR1 blocking antibody (α-IFNAR1) or IgG1 Isotype control (IC) on day −2 (d−2) before allo-HSCT. Pooled data from 3 (D) and 6 (E) independent experiments. (F) Leukocyte infiltration into the small intestine lamina propria of BALB/c mice after TBI (9Gy), with or without treatment with 3pRNA on day −1 (d−1) before TBI, IFNaR1 blocking antibody (α-IFNAR1) or IgG1 Isotype control (IC) on day −2 (d−2) before TBI, analyzed by flow cytometry. Pooled data of 2 independent experiments. (G) Survival and weight loss in BALB/c mouse recipients lacking IL-22 (II-22^{-/-}) after allo-HSCT with bone marrow (BM) and T cells from C57BL/6 wild-type (WT) mice, with or without treatment with 3pRNA on day −1 (d−1) before allo-HSCT. Pooled data from 2 independent experiments. Animal numbers per group (n) are depicted above panels. 3pRNA and α-IFNaR1 antibody treatment time points are indicated. All experiments were analyzed using two-tailed unpaired t test or ordinary one-way Anova for multiple comparisons. Survival was analyzed using the Log-rank test. Significance was set at p values <0.05, p<0.01 and p<0.001 and was then indicated with asterisks (*, ** and ***). Data are presented as mean±S.E.M.

FIGS. 5A-5F show that RIG-I-induced type I IFN signaling in non-hematopoietic cells promotes regeneration of the intestinal stem cell compartment. (A) Weight loss after allo-HSCT: 5×10⁶ bone marrow cells plus 5×10⁶ T cells of BALB/c donor mice transplanted into C57BL/6 recipient mice (genotypes are indicated in the figure) in the presence and absence of 3pRNA on day −1 (d −1). Pooled data from 2 independent experiments. (B) Organoid numbers after 5 days in culture from C57BL/6 IFNAR1^{+/+} or IFNAR1^{-/-} mice with or without the addition of 3pRNA (2 μg/ml) on day 1 of culture. One representative experiment is shown of 3 independent experiments. (C) Representative images of organoids from C57BL/6 wild-type (WT) mice after 5 days in culture with or without the addition of 3pRNA (2 μg/ml) on the first day of culture. Organoid area is shown. (D) Measurement of organoids from C57BL/6 wild-type (WT) mice after 5 days in culture with or without the addition of 3pRNA (2 μg/ml) or α-IFNaR1 blocking antibody (10 ug/ml) on the first day of culture. One representative experiment of 3 independent experiments. (E) Organoid size from C57BL/6 wild-type (WT) mice after 7 days in culture with or without the addition of recombinant murine IFN-β (20 U/ml) on day 1 of culture. One representative experiment of 3 independent experiments. (F) qPCR of Reglllγ expression in organoids 24 hours after stimulation with indicated combinations of 3pRNA, α-IFNaR1 blocking antibody and IgG1 Isotype control (IC). Pooled data from 3 independent experiments. All experiments were analyzed using two-tailed unpaired t test or ordinary one-way Anova for multiple comparisons. Survival was analyzed using the Log-rank test. Significance was set at p values <0.05, p<0.01 and p<0.001 and was then indicated with asterisks (*, ** and ***). Data are presented as mean±S.E.M.

FIGS. 6A-6E show that RIG-I activation protects intestinal stem cells after allo-HSCT. (A) qPCR expression of Lysozyme P and Lgr5 in small intestine biopsies from MAVS^{+/+} or MAVS^{-/-} C57BL/6 recipient mice after allo-HSCT with 5×10⁶ bone marrow cells and 2×10⁶ T cells from BALB/c wild-type (WT) donor mice. Pooled data from 5 independent experiments. (B) Analysis of allo-HSCT BALB/c recipients in the presence or absence of 3pRNA treatment on day −1 (d−1). Immunohistochemistry showing lysozyme staining of the small intestine of BALB/c recipients 8 days after allo-HSCT. Lysozyme-positive gut Paneth cells are indicated by black arrowheads. Histogram shows number of Paneth cells per crypt. Representative images and pooled data from 3 independent experiments. (C) qPCR showing expression of Lysozyme P and Lgr5 in intestinal epithelial cells from small intestine of Balb/c recipients 8 days after allo-HSCT, with or without treatment with 3pRNA on day −1 (d−1) before allo-HSCT. Pooled data from 3 independent experiments. (D) qPCR of Lysozyme P and Lgr5 in the small intestine of BALB/c recipients after allo-HSCT with or without 3pRNA treatment on day +1 (d +1). Data from one experiment. (E) Number of organoids harvested from C57BL/6 recipient mice on day 8 (d8) after allo-HSCT with or without treatment of the mouse with 3pRNA on day −1 (d −1) before allo-HSCT. Pooled data from 4 independent experiments. Animal numbers per group (n) are depicted above panels. All experiments were analyzed using two-tailed unpaired t test or ordinary one-way Anova for multiple comparisons. Significance was set at p values <0.05, p<0.01 and p<0.001 and was then indicated with asterisks (*, ** and ***). Data are presented as mean±S.E.M.

FIGS. 7A-7I show that STING pathway protects mouse recipients from GVHD after allo-HSCT. (A) Survival of cohoused C57BL/6 wild-type (WT) or Sting^{gt/gt} mice which received allo-HSCT from wild-type (WT) B10.BR donor mice with 5×10⁶ bone marrow cells plus 1×10⁶ T cells. Pooled data from 2 independent experiments. (B) Relative abundance of bacterial genera in the intestinal microbiota of cohoused WT and Sting^{gt/gt} mice. One representative experiment of 2 independent experiments. (C) Survival and (D) weight loss of C57BL/6 mouse recipients after allo-HSCT from B10.BR donor mice in the presence or absence of interferon stimulatory DNA (ISD) treatment on day −1 (d−1). Pooled data of 2 (C) or 3 (D) independent experiments. (E) FITC-dextran concentrations in plasma of BALB/c mouse recipients after allo-HSCT from C57BL/6 donors in the presence or absence of calf thymus DNA (CT DNA) or interferon stimulatory DNA (ISD) treatment on day −1 (d−1). Pooled data from 2 independent experiments. (F) Number of organoids harvested from WT or Sting^{gt/gt} C57BL/6 mouse small intestine after 5 days in culture with or without the addition of interferon stimulatory DNA (ISD 2 μg/ml) on the first day of culture. Pooled data from 3 independent experiments. (G) Organoids harvested from C57BL/6 mouse small intestine. Measurement of organoid size after 5 days in culture in the presence or absence of interferon stimulatory DNA (ISD 2 μg/ml), α-IFNaR1 blocking antibody (10 ug/ml) or IgG1 Isotype control (IC) added on the first day of culture. The experiment was performed 3 times and images of one representative experiment are shown. (H) qPCR of Reglllγ expression in organoids harvested from C57BL/6 wild-type mice 24 hours after stimulation with indicated combinations of interferon stimulatory DNA (ISD), α-IFNaR1 blocking antibody and IgG1 Isotype control (IC). Pooled data from 3 independent experiments. (I) DNA in plasma of BALB/c mice 24 hours after TBI (9Gy) or allo-HSCT. Pooled data from 3 independent experiments. Animal numbers per group (n) are depicted above panels. All experiments were analyzed using two-tailed unpaired t test or ordinary one-way Anova for multiple comparisons. Survival was analyzed using the Log-rank test. Significance was set at p values <0.05, p<0.01 and p<0.001 and was then indicated with asterisks (*, ** and ***). Data are presented as mean±S.E.M.

FIGS. 8A-8E shows that Endogenous RIG-I/MAVS signaling reduces intestinal tissue damage by conditioning therapy and attenuates GVHD. (A) LPL isolated from small intestines of Mavs^{+/+}/Mavs^{+/-} and Mavs^{-/-} mice were analyzed by flow cytometry on day 3 after treatment with doxorubicin (20 mg/kg). Pooled data of 3 independent experiments. Animal numbers per group (n) are depicted. (B) Weight loss of Mavs^{+/+} and Mavs^{-/-} animals after TBI+5×10⁶ BM alone or BM with 2×10⁶ T cells (donor BALB/c into recipient C57BL/6). Pooled data of 4 independent experiments. Animal numbers per group (n) are depicted. (C) Weight loss on day 8 after allo-HSCT of Mavs^{+/+} and Mavs^{-/-} animals after TBI+5×10⁶ BM alone (donor BALB/c into recipient C57BL/6). Pooled data of (B) and one additional independent experiment. Animal numbers per group (n) are depicted. (D) FITC-dextran concentrations in the serum of Mavs^{+/+} and Mavs^{-/-} recipients on d7 after TBI+5×10⁶ BM alone (donor BALB/c into recipient C57BL/6). Animal numbers per group (n) are depicted. (E) Weight loss of Rig-I^{-/+} and Rig-I^{-/-} animals after TBI+5×10⁶ BM alone or BM with 1×10⁶ T cells (donor C57BL/6 into recipient 129/sv). Animal numbers per group (n) are depicted. Experiments were analyzed using two-tailed unpaired t test. Significance was set at p values <0.05, p<0.01 and p<0.001 and was then indicated with asterisks (*, ** and ***). Data are presented as mean±S.E.M.

FIGS. 9A-9C show that donor-derived T cells show enhanced allo-reactivity in Mavs^{-/-} allo-HSCT recipients. (A) CD45.2⁺ C57BL/6 recipients (n=3) received TBI (11Gy) and 5×10⁶ syngeneic CD45.1⁺ BM and were analyzed 43 days after BMT and compared to CD45.2⁺ animals that did not receive TBI and BMT (n=3). Shown is the analysis of small intestine live lamina propria leukocytes (LPL) of one representative animal per group. (B) Mavs^{+/+} and Mavs^{-/-} littermates received TBI+5×10⁶ BM cells and 15×10⁶ CFSE labeled T cells (donor BALB/c into recipient C57BL/6). On day 4 after allo-HSCT splenic cells were analyzed by flow cytometry to identify proliferating CFSE labeled donor T-cells. Pooled data of 2 independent experiments. Animal numbers per group (n) are depicted. (C) LPL isolated from small intestines of Mavs^{+/+} and Mavs^{-/-} mice that received TBI+5×10⁶ BM cells and 2×10⁶ T cells (donor BALB/c into recipient C57BL/6) were analyzed on day 8 after allo-HSCT by flow cytometry. Pooled data of 2 independent experiments. Animal numbers per group (n) are depicted. All experiments were analyzed using two-tailed unpaired t test. Significance was set at p values <0.05, p<0.01 and p<0.001 and was then indicated with asterisks (*, ** and ***). Data are presented as mean±S.E.M.

FIGS. 10A-10J show that RIG-I ligands have to be applied before or during allo-HSCT to exert their protective effects and do not impact on GVL. (A) Weight loss of animals after 9Gy TBI+5×10⁶ BM alone with 1×10⁶ T cells (donor C57BL/6 into recipient BALB/c). Indicated mice were either left untreated or treated with 3pRNA on day −1. Pooled data of 4 independent experiments. Animal numbers per group (n) are depicted. (B) Histopathological analysis of crypt apoptosis of small intestines from allo-HSCT recipients (donor C57BL/6 into recipient BALB/c). Indicated mice were either left untreated or treated with 3pRNA on day −1. Pooled data of 3 independent experiments. Animal numbers per group (n) are depicted. (C) Survival and weight loss of allo-HSCT recipients (donor C57BL/6 into recipient BALB/c). Indicated mice were either left untreated or treated with 3pRNA on day 0 or d+1. Pooled data of 3 independent experiments. Animal numbers per group (n) are depicted. (D) Survival and weight loss of allo-HSCT recipients (donor C57BL/6 into recipient BALB/c, 9Gy TBI+5×10⁶ BM+1×10⁶ T cells). Indicated mice were left untreated or treated with 3pRNA or non-triphosphorylated control RNA (synRNA) on day −1. Pooled data of 2 independent experiments. Animal numbers per group (n) are depicted. (E) Measurement of serum cytokines of BALB/c mice on day 3 after TBI (9 Gy) using cytometric bead array (CBA). Indicated mice were left untreated or treated with 3pRNA on d−1. Pooled data of 3 independent experiments. Animal numbers per group (n) are depicted. (F) Weight loss of WT mice (C57BL/6) receiving doxorubicin (20 mg/kg). Indicated animals were treated with 3pRNA on d−1 or were left untreated. Pooled data of 6 independent experiments. Animal numbers per group (n) are depicted. (G) BALB/c mice received TBI+5×10⁶ BM cells and 15×10⁶ CFSE labeled T cells (donor C57BL/6 into recipient BALB/c). On day 4 after allo-HSCT splenic cells were analyzed by flow cytometry to identify proliferated CFSE labeled donor T cells. Shown is one representative of 2 independent experiments. Animal numbers per group (n) are depicted. (H) Lamina propria leukocytes (LPL) isolated from small intestines of BALB/c mice on day 8 after allo-HSCT (donor C57BL/6 into recipient BALB/c) were analyzed by flow cytometry. Indicated mice were left untreated or treated with 3pRNA on d−1. Pooled data of 3 independent experiments. Animal numbers per group (n) are depicted. (I) Survival of BALB/c mice that received 8.5Gy TBI+5×10⁶ BM alone or 5×10⁶ BM and 0.5×10⁶ T cells (donor C57BL/6 into recipient BALB/c) and that were inoculated with 0.25×10⁶ A20 tumor cells. Pooled data of 2 independent experiments. Animal numbers per group (n) are depicted. (J) Allo-HSCT recipients were inoculated with A20-TGL and in vivo bioluminescence imaging was conducted to determine tumor burden. Bioluminescence of one representative experiment on d21 after allo-HSCT is shown. All experiments were analyzed using two-tailed unpaired t test or ordinary one-way Anova for multiple. Survival was analyzed using the Log-rank test. Significance was set at p values <0.05, p<0.01 and p<0.001 and was then indicated with asterisks (*, ** and ***). If not otherwise indicated, significance was calculated compared to untreated groups. Data are presented as mean±S.E.M.

FIGS. 11A-11E show that S4. RIG-I-induced treatment effects are mediated by IFN-Is. (A) Serum Type I IFN levels of untreated or 3pRNA treated BALB/c WT mice were determined 4 h after i.v. injection of 25 ug 3pRNA or vehicle control (jetPEI). Animal numbers per group (n) are depicted. (B) Left panel: Albino C57BL/6 mice carrying an IFN-β^{Δβ-luc} allele were injected i.v. with 25 μg 3pRNA. 24 hours later, luciferin was injected i.v., and luciferase activity of isolated intestines was determined by bioluminescence imaging (one representative image is shown). Right Panel: The luciferase activity was quantified in a region of interest covering the small intestine (n=4 mice in untreated group; n=3 mice in the 3pRNA treated group). One representative experiment is shown. (C) Rig-I mRNA transcript expression (left panel) and M×1 mRNA transcript expression (right panel) was determined in small intestinal epithelial cells of untreated or 3pRNA-treated BALB/c mice at the indicated time points. Relative transcript levels were normalized to the housekeeping gene β-Actin. Shown are pooled data of 2 independent experiments. Animal numbers per group (n) are depicted. (D) Heatmap depicting interferon regulated genes of Balb/c mice that were solely irradiated (9Gy) (n=3, left lane), (ii) pretreated with 3pRNA prior (d−1) to irradiation (n=3, middle lane) or (iii) pre-treated with 3pRNA (d−1)+α-IFNaR1 blocking antibody (d−2) prior to irradiation (n=3; right lane). RNA from small intestines was isolated 12 h after irradiation and used for RNA sequencing. The heatmap shows all genes listed in the interferome database that show significantly changed gene expression of 3pRNA pretreated and irradiated mice compared to both the other groups simultaneously. (E) Weight loss of allo-HSCT recipients. Indicated mice received 3pRNA on d−1 and/or α-IFNaR1 blocking. Both upper and lower panel show pooled data of the same 3 independent experiments. The lower panel shows mice that received combination treatment of 3pRNA and α-IFNaR1 blocking Ab at indicated time points. Animal numbers per group (n) are depicted. All experiments were analyzed using two-tailed unpaired t test or ordinary one-way Anova for multiple comparisons. Survival was analyzed using the Log-rank test. Significance was set at p values <0.05, p<0.01 and p<0.001 and was then indicated with asterisks (*, ** and ***). If not otherwise indicated, significance was calculated compared to untreated groups. Data are presented as mean±S.E.M.

FIGS. 12A-12G show that RIG-I-induced IFN-Is enhance epithelial regeneration through stimulation of the intestinal stem cell compartment. (A) 90 days after syngeneic bonemarrow transplantation between Ifnar1^{-/-} and Ifnar1^{+/+} mice (C57BL/6), bonemarrow chimera were lethally irradiated and transplanted with 5×10⁶ BM cells with 1×10⁶ T cells from donor B10.BR mice and monitored for survival. Indicated mice were either left untreated or treated with 3pRNA on d−1. Pooled data of 2 independent experiments. Animal numbers per group (n) are depicted. (B) Survival of either Ifnar1^{fl/fl} or Ifnar1^{fl/fl} CD11cCre allo-HSCT recipients (donor BALB/c into recipient C57BL/6) in the presence or absence of 3pRNA (d−1). Pooled data of 2 independent experiments. Animal numbers per group (n) are depicted (C) Measurement of organoid size (perimeter) of C57BL/6 small intestinal organoids after 5 days in culture. Indicated crypts were treated with 3pRNA (2 μg/ml), α-IFNaR1 blocking Ab (10 ug/ml) or IgG1 Isotype control (IC). The experiment was performed 3 times and one representative experiment is shown. (D) IFN-β mRNA transcript expression 24 hours after 3pRNA stimulation of Mavs^{+/+} or Mavs^{-/-} small intestinal organoids. The experiment was performed 3 times and resulting data were pooled. (E) Measurement of organoid size (perimeter) of C57BL/6 small intestinal organoids after 5 days in culture. Indicated crypts were treated with rec. IFN-β (20 U/ml). The experiment was performed 3 times and one representative experiment is shown. (F) Number of organoids of C57BL/6 small intestinal organoids after 7 days in culture. Indicated Crypts were treated with rec. IFN-β (20 U/ml). (G) Number of C57BL/6 small intestinal organoids treated as in (C) after 7 days in culture. Survival was analyzed using the Log-rank test. All other experiments were analyzed using two-tailed unpaired t test or ordinary one-way Anova for multiple comparisons. Significance was set at p values <0.05, p<0.01 and p <0.001 and was then indicated with asterisks (*, ** and ***). Data are presented as mean±S.E.M.

FIGS. 13A-13C shows that MAVS-deficient mice do not display an inherent defect in organoid formation or in the number of Paneth cells. (A) Number of organoids grown ex vivo from small intestinal crypts of Mavs^{+/+} and Mavs^{-/-} mice after 5 days in culture. Pooled data of 7 independent experiments. Animal numbers per group (n) are depicted. (B) Determination of Lysozyme⁺ paneth cells per crypt in the ileum of untreated Mavs^{+/+} and Mavs^{-/-} mice using immunohistochemistry (IHC). Pooled data of 2 independent experiments. Animal numbers per group (n) are depicted. (C) RNA of small intestines from Balb/c WT mice isolated 24 hours after irradiation (9Gy). Gene expression was determined by qPCR. Relative transcript levels of Lysozyme P and Lgr5 were normalized to the housekeeping gene β-Actin. Pooled data of 2 independent experiments. Animal numbers per group (n) are depicted. All experiments were analyzed using two-tailed unpaired t test or ordinary one-way Anova for multiple comparisons or. Significance was set at p values <0.05, p<0.01 and p<0.001 and was then indicated with asterisks (*, ** and ***). Data are presented as mean±S.E.M.

FIGS. 14A-14G show that total body irradiation and interferon stimulatory DNA induce a systemic IFN-I response and feces-derived RNA triggers a RIG-I-dependent IFN-I response in intestinal epithelial cells. (A) Serum levels of IFN-α and IFN-β were determined at the indicated time points (hours) after i.v. injection of 50 μg interferon stimulatory DNA (ISD) into C57BL/6. Pooled data of n=2 independent experiments (n=2-5 per time point). (B) Serum levels of IL-6 (left panel) and TNF-α (right panel) of untreated or irradiated (9Gy), ISD or TBI+ISD stimulated BALB/c mice. Serum levels of the indicated proteins were determined by cytometric bead array (CBA). Pooled data of 2 independent experiments. Animal numbers per group (n) are depicted. (C) Left panel: Albino C57BL/6 mice carrying an IFN-β^{Δβ-luc} allele received TBI (11Gy). 24 hours later, luciferin was injected i.v. and luciferase activity of isolated intestine was determined. Right Panel: The luciferase activity was quantified in a region of interest covering the small intestine (n=4 mice in untreated group; n=3 mice in the TBI group). (D) Rig-I mRNA transcript expression and (E) IFN-β protein expression in murine MODE-K cells 18 hours after treatment with feces-derived RNA or 3pRNA. Number of pooled experiments depicted. (F) Ifn-β mRNA transcript expression 18 hours after stimulation of control- or Rig-I-siRNA transfected murine MODE-K cells with mouse feces-derived RNA or 3pRNA. Number of pooled experiments depicted. (G) RIG-I protein expression in control- or Rig-I-siRNA transfected murine MODE-K cells was analyzed using western blot. Number of pooled experiments depicted. The experiments were analyzed using two-tailed unpaired t test or ordinary one-way Anova for multiple comparisons. Significance was set at p values <0.05, p<0.01 and p<0.001 and was then indicated with asterisks (*, ** and ***). Data are presented as mean±S.E.M.

### DETAILED DESCRIPTION

All patents, publications, published applications and other references cited herein are hereby incorporated in their entirety into the present application.

Generally, unless otherwise specified, terms used herein are intended to be interpreted consistently with the meaning of those terms as they are known to those of skill in the art.

The term “treatment-associated” when used to characterize inflammation, GVHD, tissue damage or injury which the disclosed method seeks to minimize, prevent or otherwise ameliorate means that the inflammation, GVHD, tissue damage or injury is the result of preconditioning treatment prior to allo-HSCT. Non-limiting examples are radiation, typically total body irradiation (TBI) and administration of a chemotherapeutic agent.

RIG-I Agonists

In practicing the disclosed method, RIG-I agonists known to those of skill in the art include: 3pRNA, e.g., in vitro transcribed 3pRNA, a selective RIG-I agonist; small endogenous non-coding RNAs (sncRNAs, U1/U2)-RIG-I agonist induced via irradiation of tumor cells; double stranded RNA, e.g. ImOI-100 (Rigontec)-minimal RNA mimic of PPP-RNA, selective RIG-I agonist; Kineta small molecule RIG-I agonist (KIN1148), RIG-I agonist; SB-9200 (Spring Bank Pharmaceuticals): not specific for RIG-I, but also NOD2); double stranded RNA MCT-465 (Multicell Technologies): not specific for RIG-I, also activates MDA5 and TLR3; double stranded RNA PolyICLC (Hiltonol)-targets both RIG-I and TLR3. Oncolytic viruses that target RIG-I signaling may also be used.

cGAS/STING Agonists

cGAS/STING agonists known to those of skill in the art include: Interferon stimulatory DNA (ISD); ADU-S100 (Aduro and Novartis): Cyclical dinucleotides (e.g. cyclic diguanylate (c-di-GMP)=>less active in human STING activation; better synthetic CDN agonist, ML RR-S2 CDA=>better human STING activation, 2′,3′ CDNs); oncolytic viruses, e.g alimogene laherparepvec (T-Vec), a recombinant engineered herpes simplex virus-1 (HSV-1)—a double-stranded DNA virus encoding granulocyte-macrophage CSF (GM-CSF); 5,6-dimethyllxanthenone-4-acetic acid (DMXAA); Dispiro diketopiperzine (DSDP).

Administration of a RIG-I agonist, cGAS/STING agonist or combination thereof is most efficacious when administered prior to allo-HSCT transplantation, for example from 72 hours (3 days before) prior to transplantation until about 1 hour or immediately prior to transplantation. In some embodiments, administration may be between 48 hours (2 days before) and immediately prior to transplantation; in another embodiment administration may be between 24 hours (1 day before) and immediately prior to transplantation; in another embodiment administration may be between 12 hours and immediately prior to transplantation.

The molecular pathways that regulate the tissue repair function of type I interferon (IFN-I) during acute tissue damage are poorly understood. Described herein is a protective role for IFN-I and the RIG-I/MAVS and STING signaling pathways during acute tissue damage in mice.

Mice lacking mitochondrial antiviral-signaling protein (MAVS) were more sensitive to total body irradiation (TBI)-induced and chemotherapy-induced intestinal barrier damage. They developed worse graft-versus-host disease (GVHD) in a preclinical model of allogeneic hematopoietic stem cell transplantation (allo-HSCT) than did wild-type mice. This phenotype was not associated with changes in the intestinal microbiota, but was associated with reduced gut epithelial integrity. Conversely, targeted activation of the RIG-I pathway during tissue injury promoted gut barrier integrity and reduced GVHD.

Recombinant IFN-I or IFN-I induced by RIG-I promoted growth of intestinal organoid cultures in vitro and expression of the antimicrobial peptide regenerating islet-derived protein 3 gamma (Reglllγ). Importantly, these findings were not confined to RIG-I/MAVS signaling, as targeted engagement of the STING (STimulator of INterferon Genes) pathway also protected gut barrier function and reduced GVHD. Consistent with this, STING-deficient animals suffered worse GVHD in the allo-HSCT model compared to wild-type mice. Overall, these data suggested that activation of either RIG-I/MAVS or STING during allo-HSCT in mice resulted in IFN-I signaling that maintained gut epithelial barrier integrity. Targeting these pathways provides a novel mechanism to prevent acute intestinal injury during allogeneic transplantation.

Previous studies have proposed a protective function of IFN-I in the setting of allo-HSCT (32) and of stromal MAVS signaling in a dextran sodium sulfate (DSS)-induced mouse model of colitis (18), but the mechanisms by which IFN-I contributes to this protection remain ill defined. Chen and coworkers used a model of low-dose DSS to induce chronic tissue damage and demonstrated that MAVS signaling in stromal cells controlled tissue homeostasis by monitoring commensal bacteria (18). However, erosive epithelial damage by DSS is an artificial experimental approach that does not mirror common clinical scenarios, in which patients suffer from tissue damage after cytotoxic chemotherapy or radiation therapy or through immune activation.

A series of genetically modified (Ddx58^{-/-}, Mavs^{-/-}, Sting^{gt/gt}) and chimeric mice were used to analyze clinically relevant models of injury to the intestinal stem-cell compartment (TBI, chemotherapy) or immune-mediated acute tissue damage (allo-HSCT/GVHD), respectively. It demonstrated the role of the RIG-I/MAVS/IFN-I and STING/IFN-I pathways for the maintenance of intestinal barrier function and prevention of GVHD. Specifically, it is shown that defective MAVS or STING signaling leads to breakdown of intestinal barrier function and increased GVHD pathology. Given that cohoused wild-type and Mavs^{-/-} or Sting^{gt/gt} mice harbored similar intestinal bacterial populations, it is unlikely that differences in bacterial composition contributed to the protective role of MAVS or STING during GVHD development, unlike what has been proposed for IFN-I-mediated control of Paneth cell function (33). Rig-I^{-/-} mouse recipients of allogeneic bone marrow and T cells similarly suffered from worse GVHD, and there was a non-significant trend towards higher mortality and more weight loss in Rig-I^{-/-} mouse recipients receiving allogeneic bone marrow only. This is reminiscent of the colitis-like phenotype of Rig-I^{-/-} mice and their increased susceptibility to DSS-induced colitis (34). However, since Rig-I^{-/-} mice seemed to be viable only on the 129/sv background, their higher susceptibility to external insults may be attributable to strain-specific differences.

Exogenous stimulation of the RIG-I and STING pathways with 3pRNA or interferon stimulatory DNA in a preventive setting (one day before allo-HSCT) promoted intestinal barrier function (as measured by FITC-dextran translocation) and Paneth cell function (measured by expression of Lysozyme P), Lgr5 marker expression and the production of mucosal homeostatic factors (expression of ltgb6 and Reglllγ), ultimately protecting the recipient from the lethal consequences of systemic GVHD. Application of RIG-I agonists one day after allo-HSCT did not result in protection and even decreased expression of Lysozyme P and Lgr5. As we also noticed reduced expression of Lysozyme P and Lgr5 in the gut post-TBI and allo-HSCT, we deduced that this lack of therapeutic efficacy of 3pRNA could at least in part be explained by the loss of targetable intestinal epithelial cells following pre-transplant conditioning. Importantly, we elucidated the temporal requirements for effective IFN-I dependent signaling: IFNAR needed to be activated at the time of tissue damage, as early blockade of IFNAR before allo-HSCT but not late blockade after allo-HSCT totally abolished the protective effect of 3pRNA. An earlier study has reported reduction of GVHD if recombinant IFN-α was applied one day prior to allo-HSCT (32). Emphasizing the non-redundant role of the RIG-I/MAVS/IFN-I pathway in epithelial protection, RIG-I ligand-mediated protection was independent of IL-22, a cytokine that enhanced intestinal barrier integrity during allo-HSCT via protection of the intestinal stem cell compartment (23, 35).

Mechanistically, IFN-I (both RIG-I−/STING-induced and recombinant IFN-β) triggered growth of primary intestinal crypt cultures, an effect that was abrogated by blocking IFNAR. Growth of these epithelial “mini-guts” relies on sufficient expansion of Lgr5⁺ intestinal stem cells that eventually give rise to transient amplifying cells and, ultimately, to mature intestinal epithelial cells. Here, we found that organoid formation capacity and production of Paneth cell-derived signals (Lysozyme P) were both reduced in Mavs^{-/-} allo-HSCT recipients compared to Mavs^{+/+} allo-HSCT recipients. In contrast, we could not detect any differences in organoid formation or Paneth cell numbers between Mavs^{+/+} and Mavs^{-/-} mice in the steady-state, suggesting that MAVS and IFN-I might exert their protective functions during acute damage by activation of the intestinal stem cell compartment. Along these lines, Sting^{gt/gt} and Ifnar1^{-/-} mice also showed defects in organoid formation.

Given that Paneth cells constitute the intestinal stem cell niche and produce factors that are critical for homeostasis of Lgr5⁺ intestinal stem cells and self-renewal in the small intestine (27, 36) including WNT, EGF and Notch ligands, accurately timed RIG-I-induced or STING-induced IFN-I signaling could modulate the production of these Paneth cell-derived signals during acute tissue damage. We observed that RIG-I ligands protected Paneth cells in allo-HSCT mouse recipients and enhanced expression of Lysozyme P and Lgr5. Moreover, RIG-I-induced and STING-induced IFN-I enhanced the production of Reglllγ that could contribute to limiting intestinal tissue damage by sustaining a protective shield against bacterial colonisation and translocation (21, 37). Finally, we found that administration of 3pRNA prior to allo-HSCT allowed retrieval of more organoids from the small intestine of treated recipients compared to untreated control recipients and required the RIG-I adaptor MAVS to induce epithelial regeneration. Engagement of RIG-I in vivo thus augmented intestinal stem cell function and epithelial regeneration during allo-HSCT. Given that expression of RIG-I, MAVS and STING have previously been identified in Lgr5⁺ intestinal stem cells in a proteomic screen (38), future studies will clarify whether endogenous RIG-I and STING ligands and IFN-I enhance organoid growth by directly acting on Lgr5⁺ intestinal stem cells. Alternatively, Bmi⁺ intestinal stem cells, considered to be injury-inducible cells with full potential for epithelial regeneration shortly after irradiation damage (39), could be targets for RIG-I/MAVS, STING or IFN-I dependent signals.

Under conditions of chronic viral challenge and chronic IFN-I signaling, myeloid cells are the main targets of IFN-I signals, controlling epithelial barrier integrity through secretion of apolipoproteins L9a/b (10). In addition, NK cells (both donor or recipient) reduce inflammation after irradiation-induced gut epithelial barrier loss and GVHD in several mouse models (40) and are activated by IFN-I after 3pRNA injection (22). Non-intestinal epithelial cell IFN-I targets could contribute to the 3pRNA-induced protection against gut barrier loss and GVHD. 3pRNA increased expression of apolipoproteins L9a/b in the small intestine of irradiated wild-type mice, an effect that was entirely dependent on IFN-I signaling. In contrast, weight loss during GVHD in Ifnar1^{fl/fl} CD11cCre mice was higher, but reduction of GVHD-associated weight loss by RIG-I activation was not affected. This suggested that although IFN-I signaling through DCs appears to be important for limiting tissue damage under certain conditions, protection from tissue injury in GVHD via RIG-I activation is not mediated by IFN-I signaling in DCs.

Our data suggested that endogenous RIG-I/MAVS and STING signaling resulted in protective IFN-I signaling to maintain epithelial barrier integrity, specifically in the context of tissue damage induced by TBI, chemotherapy and GVHD. In this respect, identifying endogenous ligands that engage these pathways and mediate protection is of particular interest.

### EXAMPLES

### Example 1: Materials and Methods

The goal of this study was to evaluate the impact of RIG-I/MAVS and STING signaling on gut integrity during acute tissue injury and GVHD. To assess this, acute tissue damage was induced by total body irradiation (TBI), cytotoxic chemotherapy and mouse models of allogeneic hematopoietic stem cell transplantation (allo-HSCT). GVHD intensity was quantified using survival, weight loss, histopathology and immunohistochemistry. Intestinal barrier function was analyzed by using FITC-Dextran translocation, expression of antimicrobial peptides and neutrophil influx into the lamina propria. Bacteremia was measured in the serum by counting colony-forming units (CFUs). Organoid cultures of small intestinal crypts were used as an indicator for epithelial regeneration. Damage-associated DNA release was quantified using total DNA isolated from mouse plasma. Quantitative PCR was performed for gene expression analysis of interferon signaling, antimicrobial peptides and small intestine stem or Paneth cell markers. 16S rRNA sequencing was performed to detect potential differences in the intestinal bacterial composition of wild-type, Mavs^{-/-} or Sting^{gt/gt} mice. For animal studies, sample sizes were chosen according to the power of the statistical test of each experiment. For all studies, animal numbers are depicted in the figures and the number of independent experiments is listed in the figure legends. Wild-type and genetically modified mice were randomized into experimental groups and randomly assigned to different cages. Experienced GVHD-pathologists performed histopathologic scoring of intestinal damage after allo-HSCT in a blinded fashion. All mouse antibodies used were validated for use with flow cytometry by the supplier, either eBioscience, BD Biosciences or BioLegend. Mouse antibodies and their clones are listed in Supplementary Table 1 (Clone number, 1DegreeBio, Reference ID). Cell lines are tested for mycoplasma at frequent intervals. We did not exclude outliers in any experiment. For all results, statistical tests are described in the figure legend.

Mice

C57BL/6 (H-2k^{b}, Thy-1.2), BALB/c (H-2k^{d}, Thy-1.2) were purchased from Janvier Labs (France). Mavs^{-/-} (C57BL/6) were provided by the late J. Tschopp. Ifnar1^{-/-} (C57BL/6) mice were provided by Joseph C. Sun (MSKCC), II-22^{-/-} (Balb/c) mice were provided by Genentech. Rig-I^{-/-} mice (129/sv) were provided by Zhu-gang Wang (State Key Laboratory of Medical Genomics, Shanghai Jiao Tong University School of Medicine, Shanghai 200025, P.R.China) (34). Ifn-β-β^{Δβ-luc} mice used for in vivo imaging were backcrossed to C57BL/6 albino background (42). Floxed Ifnar1 mice (C57BL/6) crossed with CD11c-Cre mice (C57BL/6) were provided by U. Kalinke (Twincore, Hannover, Germany). Sting^{gt/gt} mice were from Jackson (Stock number 017537). Mice were used between 6 and 12 weeks of age at the onset of experiments and were maintained in specific pathogen free conditions. We used littermates derived from heterozygous breeding pairs (Mavs^{-/-}, Mavs^{+/+}; Ifnar1^{fl/fl} CD11c-Cre⁺, Ifnar1^{fl/fl} CD11c-Cre⁻; Rig-I^{-/-}, Rig-I^{+/-}) or cohoused m ice as indicated in the results or figure legends. Animal studies were approved by the local regulatory agencies (Regierung von Oberbayern, Munich, and Landesamt für Verbraucherschutz and Lebensmittelsicherheit (LAVES), Oldenburg, Germany) and by the Memorial Sloan-Kettering Cancer Center (MSKCC) Institutional Animal Care and Use Committee (IACUC).

Bone Marrow Transplantation (BMT) Model

Allogeneic bone marrow transplants were performed as previously described (43). Briefly, recipients were given 5×10⁶ BM cells directly after lethal total body irradiation (TBI) with 2×4.5Gy (BALB/c), 2×5.5Gy (C57BL/6) or 2×5Gy (129/sv). T cell doses (CD4/CD8 or CD5 MACS enrichment, Miltenyi) varied depending on the transplant model: Donor C57BL/6 into recipient BALB/c (0.5×10⁶ or 1×10⁶ when indicated), donor BALB/c into recipient C57BL/6 (2×10⁶), donor C57BL/6 into recipient 129/sv (1×10⁶), donor B10.BR into C57BL/6 (1×10⁶). We used T cell depleted BM in all allo-HSCT experiments with BM only controls. T cell depletion of BM cells was performed as previously described (44).

Generation of Chimeric Recipients with Mavs or Ifnar1 Deficiency of Hematopoietic or Non-Hematopoietic Tissues

WT, Mavs^{-/-} and Ifnar1^{-/-} recipients (C57BL/6J) were injected as syngeneic bone marrow transplantation (BMT) with 5×10⁶ WT or Mavs^{-/-} or Ifnar1^{-/-} BM cells (C5761/6) intravenously directly after TBI with 2×5.5Gy. Between 45 and 90 days after first syngeneic BMT, allogeneic HSCT (donor BALB/c into recipient C5761/6: T cell dose 2×10⁶, TBI 2×5.5Gy; donor B10.BR into recipient C57BL/6: T cell dose 1×10⁶, TBI 2×4.5 Gy) was performed.

In Vivo Permeability Assay

FITC-dextran assay was performed as previously described (35). Mice were kept without food and water for 8 hours and then FITC-dextran (# FD4-1G, Sigma) was administered by oral gavage at a concentration of 50 mg/ml in water (750 mg/kg). 4.5 h later, plasma was collected from peripheral blood (8800rcf, 10 min), then mixed 1:1 with PBS and analyzed on a plate reader at an excitation wavelength of 485 nm and an emission wavelength of 535 nm.

Determination of Bacteremia

To determine bacteremia, peripheral blood was collected and centrifuged at 400 g for 5 min, supernatant (blood plasma) was collected, plated and incubated at 37° C. under anaerobic conditions using Columbia Agar plates. After 48 h CFUs were counted and bacteremia was quantified in CFUs per ml blood plasma.

Isolation of Lamina Propria Leukocytes and Intestinal Epithelial Cells (IEC) from the Small Intestine

Isolation was performed as previously described (43). Briefly, Peyer's patches were excised from ileum (defined as distal 1/3 of small intestine) and ileums were flushed with cold PBS and cut into 2 cm pieces. Longitudinally opened intestines were washed and incubated with HBSS solution containing 2 mM EDTA, 10 mM HEPES, 10% FCS (Hyclone), 1% Penicillin-Streptomycin, 1% L-Glutamine and 1 mM DTT (all Sigma-Aldrich). After incubation on a shaker (225 rpm) at 37° C. for 2×15 min, tissues were washed and filtered through a 100 μm strainer (BD 352360). The flow-through were centrifuged for 5 min at 1,500 r.p.m and the remaining pellet was lysed in TRIzol (Ambion) for subsequent RNA extraction. Next, intestines were incubated for 45 min in PBS^{+Ca+/+Mg} supplemented with FCS (10%), Collagenase 11 (200 U/ml; Worthington), and DNase I (0.05 mg/ml; Roche) on a shaker at 37° C. Lamina Propria Leukocytes (LPL) in suspension were then purified on a 40/80% Percoll gradient (Biochrom).

In Vivo Analysis of Neutrophil Infiltration

Phenotypical analysis of neutrophils was performed as previously described (45). For assessment of neutrophil infiltration after TBI or doxorubicin treatment, 6-12 weeks old mice were irradiated with 9Gy (Balb/c) or 11Gy (C57BL/6) or treated with doxorubicin injected intraperitoneally (i.p.) (7.5 mg/KG body weight, unless indicated otherwise). On day 3 after intervention mice were sacrificed, Lamina Propria Leukocytes were isolated, counted and neutrophils within the LPLs were analyzed by flow cytometry and normalized to the absolute number of averagely isolated cells (1×10⁶).

Flow Cytometry

Cell suspensions were stained in PBS with 3% FCS. Fluorochrome-coupled antibodies were purchased from eBioscience or BioLegend and are listed in Table 1. For intracellular cytokine staining (ICS), T cells were activated with 80 nM Phorbol-12-myristat-13-acetat (PMA; Sigma), 1 μM ionomycin (Merck Millipor) and Brefeldin A for 4 hours. For ICS, the Foxp3 Transcription Factor Fixation/Permeabilization Kit (eBioscience) was used according to manufacturer's instructions. Data were acquired on a FACS Canto II (BD Biosciences) and analyzed using FlowJo software (TreeStar).

Analysis of T Cell Proliferation In Vivo

In vivo T cell analysis was performed as previously described (46). T cell and BM preparation was performed as described above. T cells were stained with 3.5 μM carboxyfluorescein diacetate succinimidyl ester (CFSE, eBioscience) for 12 minutes at 37° C., washed and counted. 15×10⁶ stained cells were transplanted into lethally irradiated allogeneic recipients as described above. Spleens were harvested on day 3 and analyzed with FACS.

Crypt Isolation

Isolation of intestinal epithelial crypts was performed as previously described (26). Briefly, after harvesting small intestines, the organs were opened longitudinally and washed. Small intestine was incubated in 10 mM ethylenediamine-tetraacetatic acid (EDTA) for 25 min (4° C.) to dissociate the crypts. The supernatant containing crypts was collected.

Organoid Culture

250 crypts per well were suspended in growth factor reduced Matrigel (Corning) (33% ENR-medium; 66% growth factor reduced Matrigel) at 4° C. Then, they were plated in delta-surface Nunc 24-well plates in 30 μL drops, each containing approximately 250 crypts. After the Matrigel drops polymerized, 500 ul complete crypt culture medium was added to small intestine crypt cultures (ENR-medium: advanced DMEM/F12 (Life technologies), 2 mM L-glutamine (Sigma), 10 mM HEPES (Life technologies), 100 U/ml penicillin/100 μg/ml streptomycin (Life technologies), 1.25 mM N-acetyl cysteine (Sigma), 1×B27 supplement (Life technologies), 1×N2 supplement (Life technologies), 50 ng/ml mEGF (Peprotech), 100 ng/ml rec. mNoggin (Peprotech), 5% human R-spondin-1 conditioned medium of hR-spondin-1-transfected HEK 293T cells). Together with the crypt culture medium, 2 μg/ml of 3pRNA or 2 μg/ml of ISD complexed with Lipofectamine 2000 (Invitrogen) or recombinant murine (rm) IFN-β (20 U/ml; PBL (12400-1)) was added. All plates were incubated at 37° C./5% CO2 and medium was replaced every 2-3 days. IFN-β was added again with every medium change. For IFNαR1 blockade, 10 ug/ml of antibody were added to the matrigel before polymerization and with every medium change (MAR1-5A3 anti-mIFNalphaR1 antibody or MOPC-21 Mouse IgG1 as isotype control (BioXCell)).

Histopathologic Analysis

Intestines were harvested 8 days after allo-HSCT or 72 hours after TBI for histopathologic assessment of intestinal tissue injury. Samples were formalin-preserved, paraffin-embedded, sectioned, and stained with hematoxylin and eosin (H&E). For evaluation of intestinal GVHD after allo-HSCT, blinded scoring was performed by experienced pathologists (C.L. or M.R.) as previously described (47). For evidence of intestinal tissue damage after TBI, tissues were examined by four established criteria in a blinded fashion by a pathologist (S.M.): crypt apoptosis (% of crypt containing at least 1 apoptotic cell), crypt abscesses (Absent (0), Present (1)), granulocytic infiltrates (Absent (0), minimal (1), mild (2), moderate (3), marked (4)) and villus atrophy (absent (0), minimal (1), mild (2), moderate (3), marked (4)). Each mouse was given an individual cumulative score (histopathology score) based on the above criteria.

Immunohistochemistry

Intestines of mice 8 days after allo-HSCT were harvested, formalin-fixed, paraffin embedded. The immunohistochemical detection of Lysozyme was performed using Discovery XT processor (Ventana Medical Systems). The tissue sections were deparaffinized with EZPrep buffer (Ventana Medical Systems), antigen retrieval was performed with CC1 buffer (Ventana Medical Systems) and sections were blocked for 30 minutes with Background Buster solution (Innovex). Slides were incubated with anti-Lysozyme antibodies (DAKO; cat # A099; 2 ug/ml) for 5 h, followed by 60 minutes incubation with biotinylated goat anti-rabbit IgG (Vector labs, cat # PK6101) at 1:200 dilution. The detection was performed with DAB detection kit (Ventana Medical Systems) according to manufacturer's instruction. Slides were counterstained with hematoxylin (Ventana Medical Systems) and coverslipped with Permount (Fisher Scientific). To quantify Lysozyme⁺ Paneth cells, the number of positive cells per crypt was evaluated over a 5000 μm length of intestinal mucosa. Lysozyme⁺ Paneth cells are depicted as mean Paneth cell number/crypt. Quantification was performed in a blinded fashion by S.M.

Detection of Bioluminescence and In Vivo Imaging

Ifn-β^{Δβ-luc} mice were injected i.v. with 100 μl luciferin (30 mg/ml in PBS)/20 g mouse weight and anesthesized using isoflurane. Within 10 min after luciferin injection, mice or isolated organs were analyzed with an in vivo imaging instrument (IVIS 200; PerkinElmer). The acquired images were analyzed using Living Image 4.4. software.

Quantitative PCR

RNA was isolated from cells lysed in TRIzol (ambion) or from whole tissue homogenates. Tissue homogenates were prepared as follows: 1 cm large or small intestine was flushed and longitudinally opened pieces were frozen in 500 ul TRIzol reagent using liquid nitrogen. After thawing, samples were supplemented with stainless stell beads 5 mm (Qiagen) and homogenized using a Tissuelyser II (Qiagen) 1 min with 30 Hz (1800 oscillations/minute). Total RNA was isolated and transcribed using standard methods and kits according to manufacturer's protocols (RNeasy Mini Kit, Qiagen; SuperScript III Reverse Transcriptase, invitrogen). The specific primer pairs were as follows: mReglllγ fwd TTCCTGTCCTCCATGATCAAAA (SEQ ID NO: 1), rev CATCCACCTCTGTTGGGTTCA (SEQ ID NO: 2); mActin fwd CACACCCGCCACCAGTTCG (SEQ ID NO: 3), rev CACCATCACACCCTGGTGC (SEQ ID NO: 4); mLgr5 fwd ACCCGCCAGTCTCCTACATC (SEQ ID NO: 5) rev GCATCTAGGCGCAGGGATTG (SEQ ID NO: 6); mLysozymeP fwd CAG GCCAAGGTCTACAATCG (SEQ ID NO: 7), rev TTGATCCCACAGGCATTCTT (SEQ ID NO: 8); mltgb6 fwd ATTGTCATTCCCAATGATGG (SEQ ID NO: 9), rev CATAGTTCTCATACAGATGGAC (SEQ ID NO: 10). The qPCR Core kit for SYBR Green I (Eurogentec) and a LightCycler 480 II (Roche) Real-Time PCR System were used as indicated by the manufacturer. The relative transcript level of each gene was calculated according to the 2-Ct, for unnormalized genes, and the 2-ΔΔCt method, for the genes normalized to β-Actin. Alternatively, the following Taqman Expression Assay IDs were used: BETA-ACTIN Mm01205647_g1; IFNB1 Mm00439552_s1; REG3G Mm00441127_m1.

Measurement of Cytokines

TNF and IL-6 were analyzed using the Cytometric Bead Array Enhanced Sensitivity Flex Set System (BD) according to manufacturer's instructions. IFNα and IFNβ were analyzed by ELISA (PBL Assay Science) according to manufacturer's instructions.

Assessment of Epithelial Regeneration in Intestinal Organoid Cultures

To determine the effect of 3pRNA/interferon stimulatory DNA/rmIFN-β on organoid size and morphology, bright-field microscopy images were taken using a Zeiss Axiovision Observer microscope with a 5× objective lens after 5 or 7 days in culture. 2D area and perimeter were analyzed using border perimeter tracing of organoids found in four representative fields of each well using Image J software. For assessment of gene expression by quantitative (q) PCR, organoids were subjected to RNA extraction 24 hours after culture using Trizol reagent (Invitrogen) according to manufacturer's protocol. Isolated RNA was reverse-transcribed using the Quantitect Reverse Transcription Kit (Qiagen). Gene expression was assessed by quantitative real-time PCR using Taqman Expression Assay pre-designed probes (Applied Biosystems). Signals were normalized to β-Actin. mRNA expression. Normalized values were used to calculate relative expression by ΔΔCt analysis or absolute expression by ΔCt. Taqman IDs are depicted below (qPCR).

Reagents

OptiMEM reduced-serum medium was from Invitrogen. Double-stranded in vitro-transcribed 3pRNA (sense, 5′-UCA AAC AGU CCU CGC AUG CCU AUA GUG AGU CG-3′ (SEQ ID NO: 11) was generated as described (22). Synthetic dsRNA with the same sequence but lacking the 5′-triphosphate (synRNA) was purchased from Eurofins (Ebersberg, Germany). Interferon stimulatory DNA was purchased from Invivogen.

Drug Treatment

Mice were treated on indicated time points with 3pRNA or interferon stimulatory DNA (25 μg if not indicated otherwise). 3pRNA or interferon stimulatory DNA was complexed in 3.5 μl in vivo-jetPEI (Polyplus) and injected intravenously. In some experiments mice were treated i.p. with 500 ug IFNaR1 blocking antibody (Clone: MAR1-5A3, BioXCell, West Lebanon, N.H.) or IgG1 Isotype control (Clone: MOPC-21, BioXCell, West Lebanon, N.H.) as indicated.

16S RNA Gene Sequencing

Stool specimens were stored at −80° C. DNA was purified using a phenol-chloroform extraction technique with mechanical disruption (bead-beating) based on a previously described protocol (48) and analyzed using the Illumina MiSeq platform to sequence the V4-V5 region of the 16S rRNA gene. Sequence data were compiled and processed using mothur version 1.34(49), screened and filtered for quality (50), then classified to the species level (51) using a modified form of the Greengenes reference database (52), screened and filtered for quality (50), then classified to the species level (51) using a modified form of the Greengenes reference database (52).

Quantification of Plasma DNA Levels

Mouse plasma was collected from peripheral blood (8800rcf, 10 min). Plasma samples of 3-4 mice were combined to a final volume of 400-500 μl and DNA extracted using the QIAamp Circulating Nucleic Acids Kit (Qiagen). dsDNA was quantified using a Qubit 2.0 Fluorometer with the Qubit dsDNA HS Assay Kit (Thermo Fisher Scientific).

GVT Model and Bioluminescence Imaging

A20-TGL (H-2^{d}), a BALB/c B-cell lymphoma, were generated as described previously (53). A20-TGL tumor cells were inoculated via separate intravenous injection on the day of allo-BMT (54). To visualize and quantify tumor burden, A20-TGL inoculated mice were administered D-luciferin (Goldbio), anesthetized, and imaged using in vivo bioluminescence imaging systems (Caliper Life Sciences)

Cell Lines, Culture and RNA Transfection, Feces RNA Isolation

Mode-K cells were purchased from Dominique Kaiserlian (French Institute of Health and Medical Research, Unit of Immunity Infection Vaccination, France) and cultured as previously described (31). Cell lines were tested as mycoplasma negative. Where indicated, MODE-K cells were transfected with mouse RIG-I siRNA (100 μM, Eurofins Genomics) or control siRNA (Qiagen) using Lipofectamine 2000 (Life Technologies) according to manufacturer's instructions. After 48 h, cells were transfected with 3pRNA (0.8 μg/mL) or mouse feces-derived RNA complexed to Lipofectamine 2000. Supernatants were collected and RNA was extracted 18 h after transfection followed by IFN-β measurement with ELISA (PBL Assay Science) or by assessment of IFN-β mRNA by qPCR. Mouse feces from healthy WT mice was diluted (RNAprotect Reagent, Quiagen) and homogenized with Glass beads (Sigma) and a Tissuelyser II (Qiagen). After centrifugation, supernatant was subtracted and total feces RNA was isolated using standard methods and kits according to manufacturers' protocols.

Gene Expression Profiling Analysis

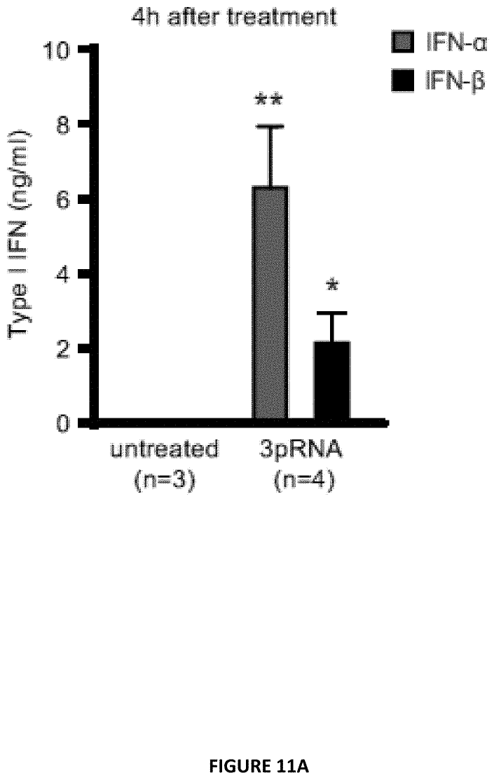

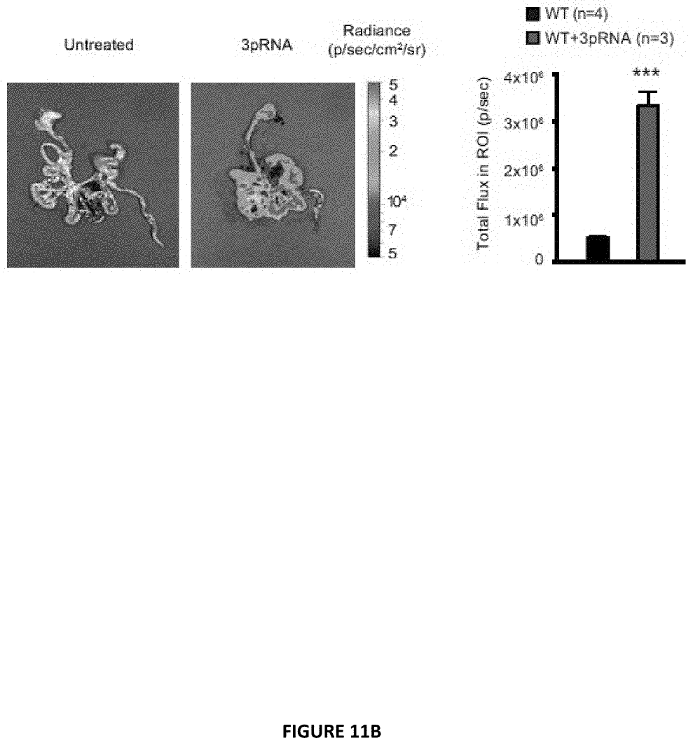

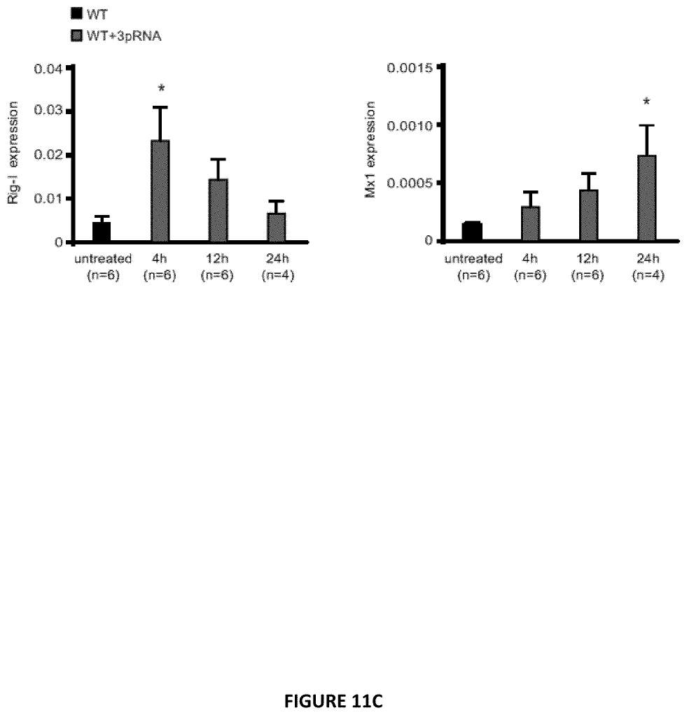

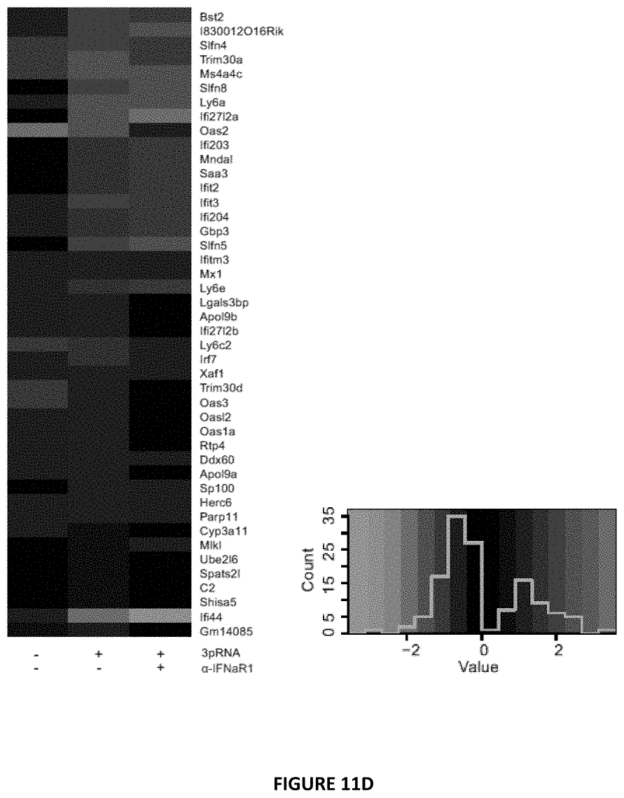

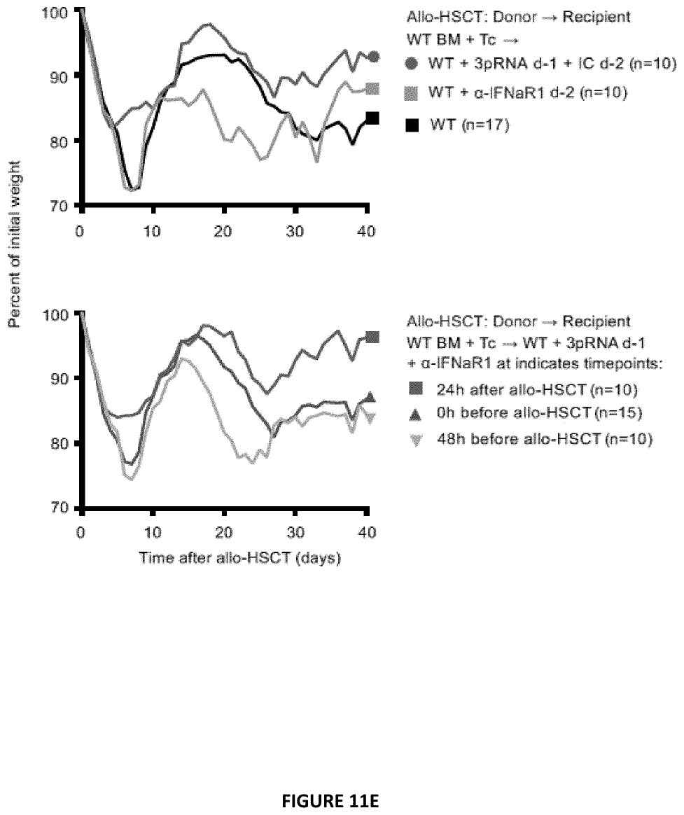

For gene expression profiling analysis, (i) Balb/c mice were solely irradiated (9Gy) (n=3), (ii) pretreated with 3pRNA prior (d−1) to irradiation (n=3) or (iii) pre-treated with 3pRNA (d−1)+α-IFNαR1 blocking antibody (d−2) prior to irradiation (n=3). RNA from small intestines was isolated 12 h after irradiation and used for RNA sequencing. Poly(A) RNA sequencing was performed with three biological replicates for each group and analyzed with an Illumina HiSeq2500 platform. The heatmap depicted in FIG. 11D shows all genes listed in the interferome database (55) that show significantly changed gene expression of 3pRNA pretreated and irradiated mice compared to both the other groups simultaneously.

Data Analysis

The output data (FASTQ files) were mapped to the target genome using the rnaStar aligner that maps reads genomically and resolves reads across splice junctions. We used the 2 pass mapping method in which the reads are mapped twice. The first mapping pass used a list of known annotated junctions from Ensemble. Novel junctions found in the first pass were then added to the known junctions and a second mapping pass was done. After mapping we computed the expression count matrix from the mapped reads using HTSeq (www-huber.embl.de/users/anders/HTSeq) and one of several possible gene model databases. The raw count matrix generated by HTSeq was then processed using the R/Bioconductor package DESeq (www-huber.embl.de/users/anders/DESeq) which was used to both normalize the full dataset and analyze differential expression between sample groups.

A heatmap was generated using the heatmap.2 function from the gplots R package. The data plot was the mean centered normalized log 2 expression of the top 100 significant genes. For simple hierarchical clustering the correlation metric was used (Dij=1−cor(Xi,Xj)) with the Pearson correlation on the normalized log 2 expression values.

Statistics

Animal numbers per group (n) are depicted in the figure legends. Technical replicates were never used. GraphPad Prism version 6 was used for statistical analysis. Survival was analyzed using the Log-rank test. Differences between means of experimental groups were analyzed using two-tailed unpaired t test or ordinary one-way Anova correspondingly to the distribution shape of our observations. We used ordinary one-way Anova for multiple comparisons and always performed Dunnett's test for Multiple-test corrections. Applied statistical tests are indicated in the figure legends. Significance was set at p values <0.05, p<0.01 and p<0.001 and was then indicated with asterisks (*, ** and ***). Data are presented as mean±S.E.M.

TABLE S1
Antibodies

| Target structure | Clone# | 1DegreeBio ID |
|---|---|---|
| CD11b | M1/70 | 1DB-001-0001021785 |
| CD11c | N418 | 1DB-001-0000839554 |
| CD3 | 17A2 | 1DB-001-0001110661 |
| CD4 | GK1.5 | 1DB-001-0000263404 |
| CD45.1 | A20 | 1DB-001-0000839250 |
| CD45.2 | 104 | 1DB-001-0000839196 |
| CD8a | 53-6.7 | 1DB-001-0000263247 |
| IFNaR1 | MAR1-5A3 | 1DB-001-0000840263 |
| IFN γ | XMG1.2 | 1DB-001-0001110823 |
| Ly-6G/Ly-6C (Gr-1) | RB6-8C5 | 1DB-001-0000839101 |

### Example 2. Endogenous RIG-I/MAVS Signaling Reduces Intestinal Tissue Damage Due to Conditioning Therapy and Attenuates GVHD in Mice

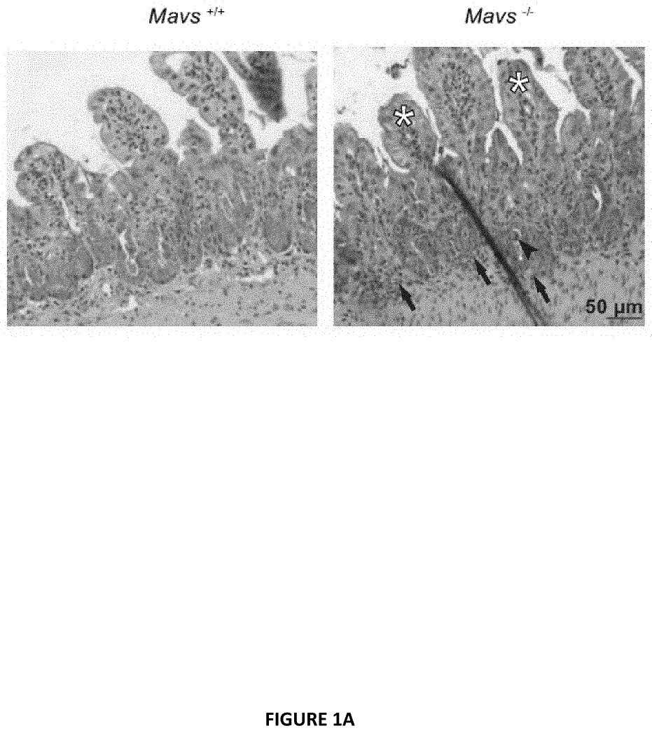

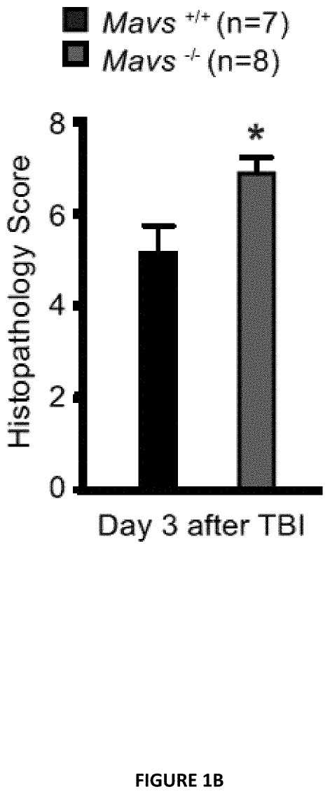

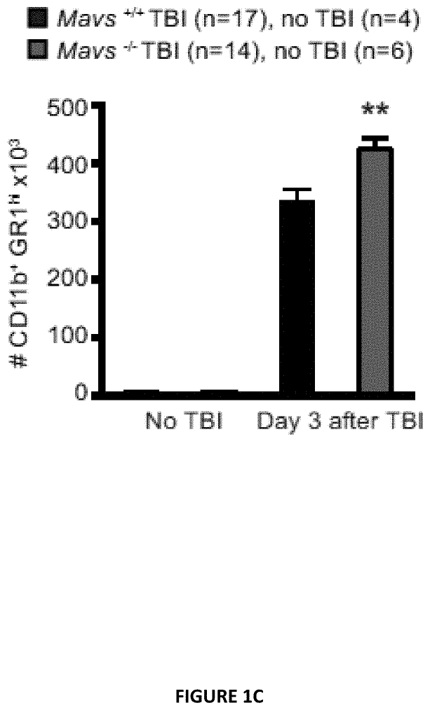

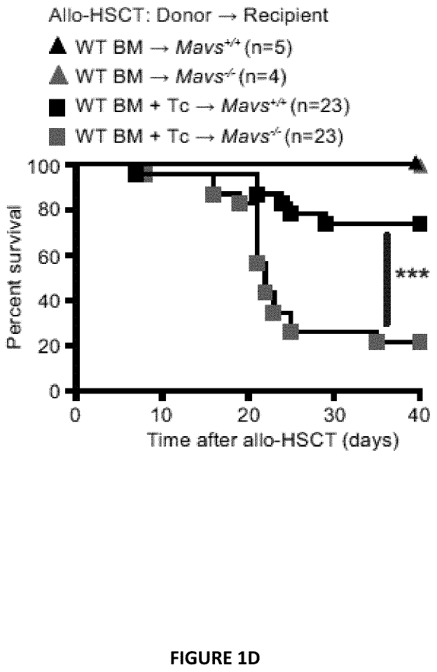

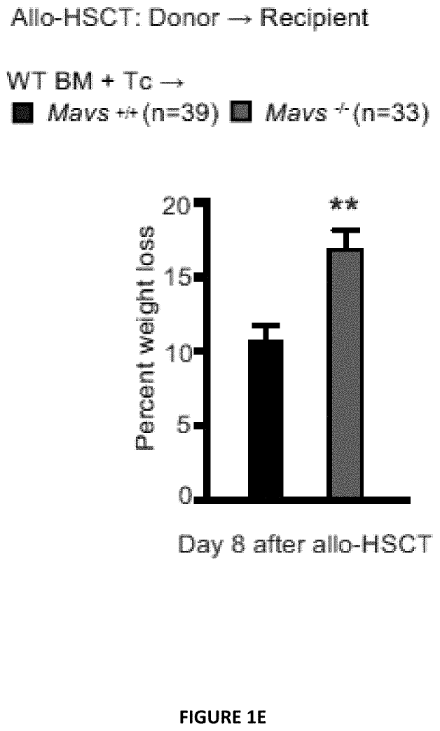

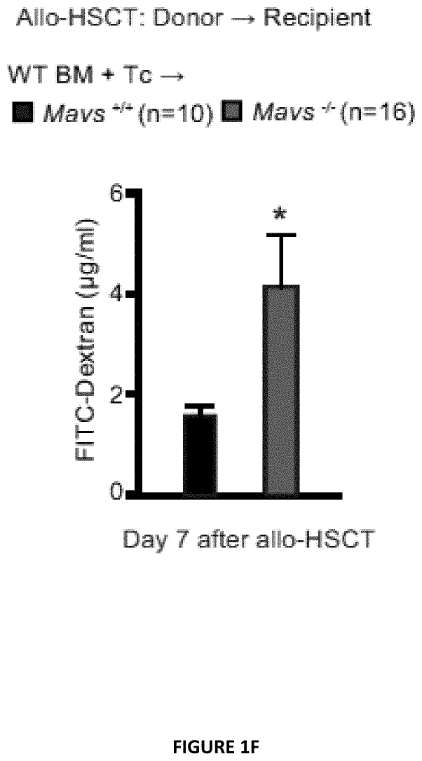

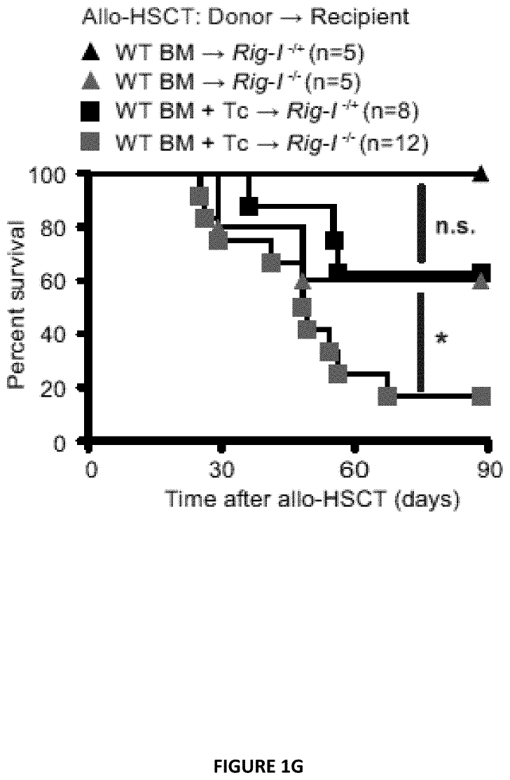

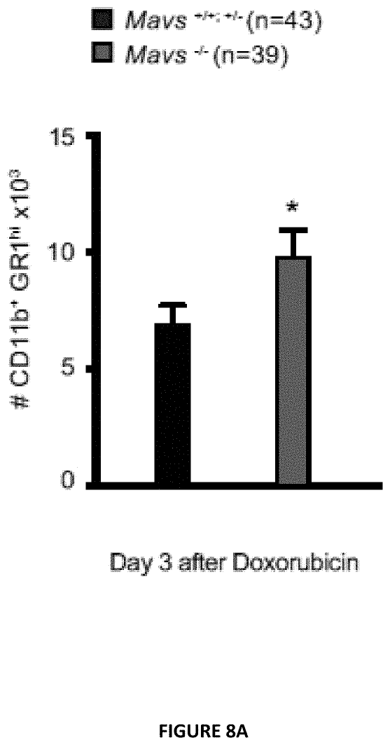

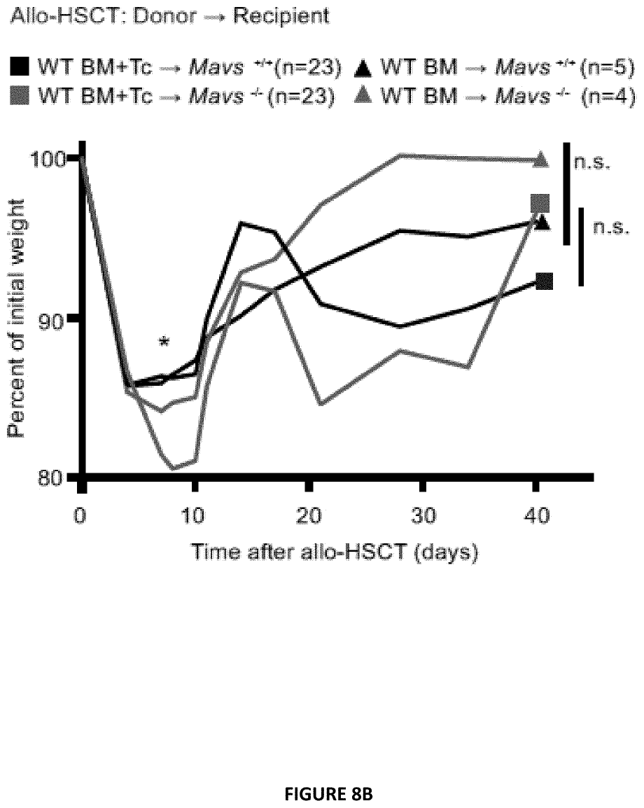

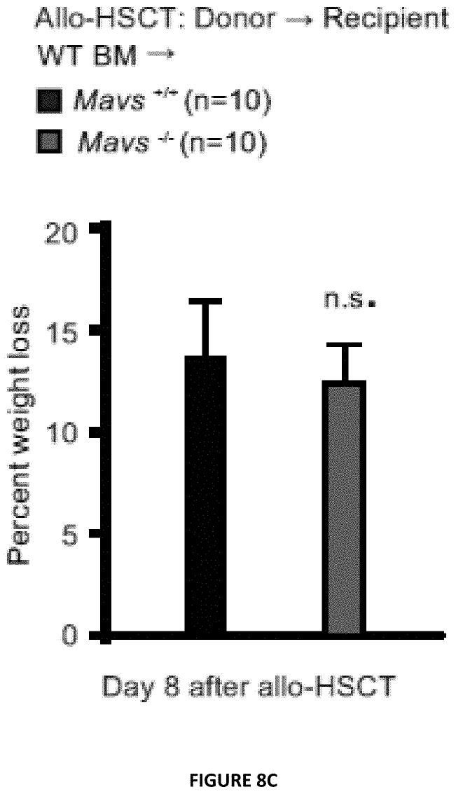

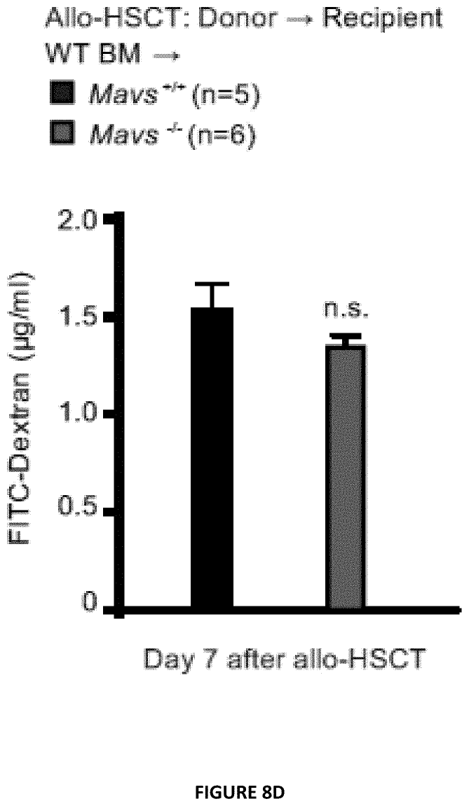

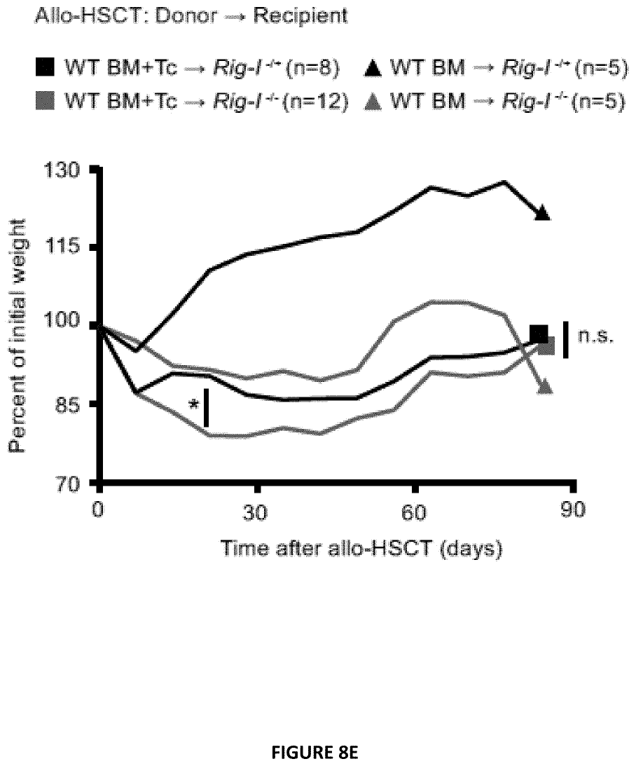

First, genotoxic tissue damage and regeneration in wild-type (WT) mice and mice genetically deficient for MAVS (Mavs^{-/-}) was assessed. Mice were exposed to lethal TBI, which causes damage to dividing cells and induces loss of intestinal epithelial barrier function (14, 15). Compared to Mavs^{+/+} littermates, Mavs^{-/-} mice exhibited worse mucosal damage in the small intestine with increased crypt apoptosis, villus atrophy, crypt abscesses and granulocytic infiltrates (FIG. 1A, B). Neutrophil influx into the gut mucosa, a surrogate marker for intestinal integrity (16), was higher in Mavs^{-/-} compared to Mavs^{+/+} littermates following TBI (FIG. 1C) or chemotherapy with doxorubicin (17) (FIG. 8A). Consequently, in an acute GVHD model where conditioning-associated intestinal damage is crucial for subsequent allogeneic T cell mediated pathology, we observed that Mavs^{-/-} recipients of allogeneic donor bone marrow and T cells had increased mortality compared to Mavs^{+/+} littermates (FIG. 1D). In addition, Mavs^{-/-} allo-HSCT recipients exhibited greater weight loss (FIG. 1E and FIG. 8B, C) and reduced intestinal barrier integrity as measured by translocation of intraluminal FITC-dextran into the systemic circulation on day 7 after HSCT (FIG. 1F and FIG. 8D). Rig-I (Ddx58)^{-/-} allo-HSCT recipients of donor bone marrow and T cells also displayed increased mortality and weight loss compared to Rig-I littermates (FIG. 1G and FIG. 8E). We observed a non-significant trend towards higher mortality and more weight loss of Rig-I^{-/-} bone marrow recipients (FIG. 1G and FIG. 8E).

### Example 3

MAVS Signaling in Non-Hematopoietic Cells Attenuates GVHD and Maintains Intestinal Barrier Function in Mice

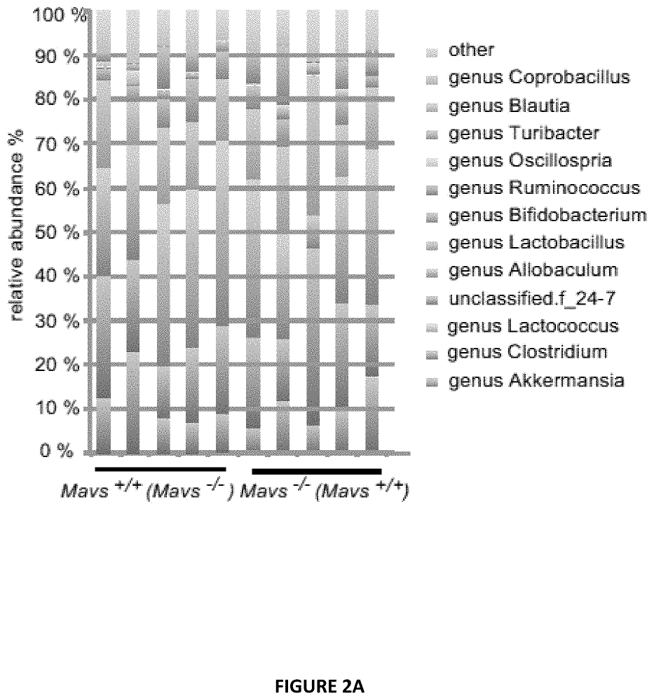

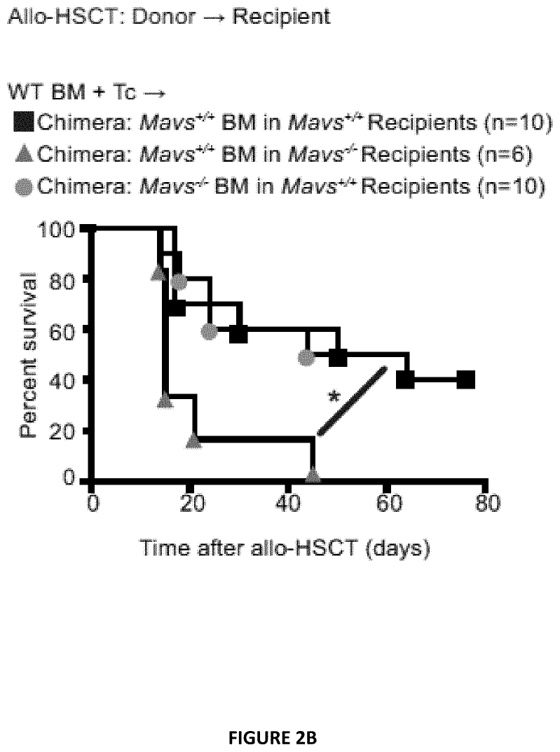

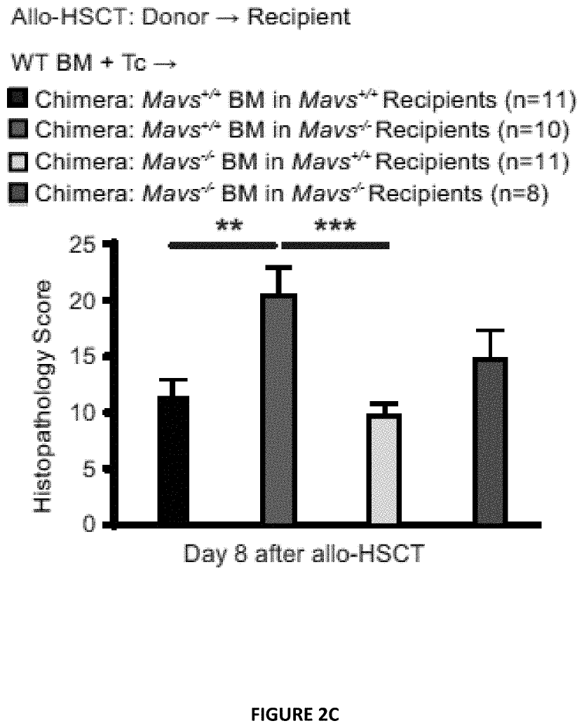

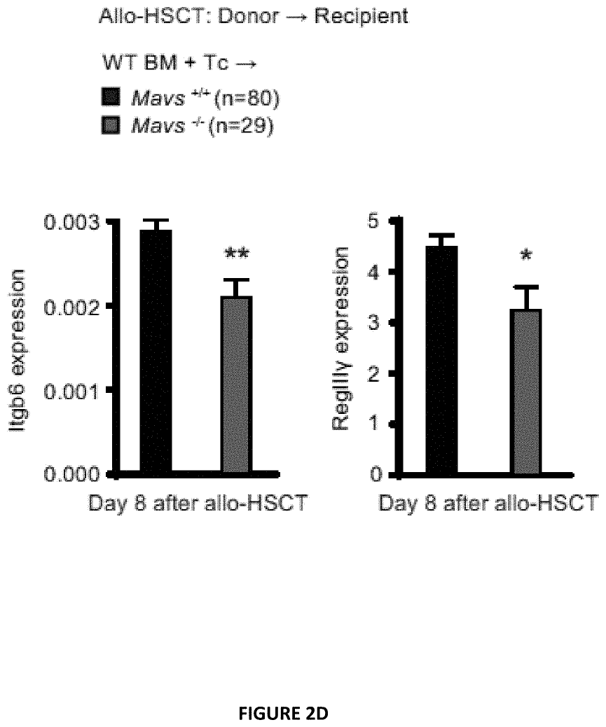

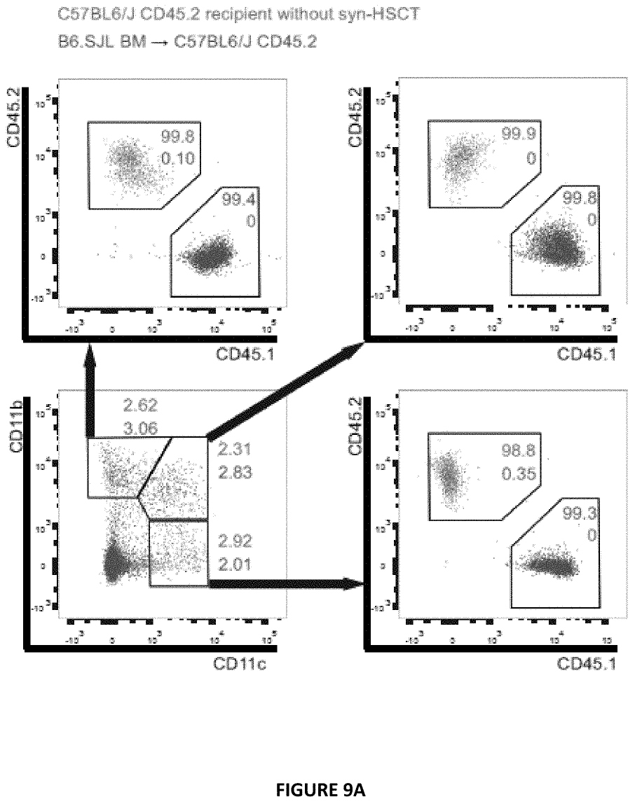

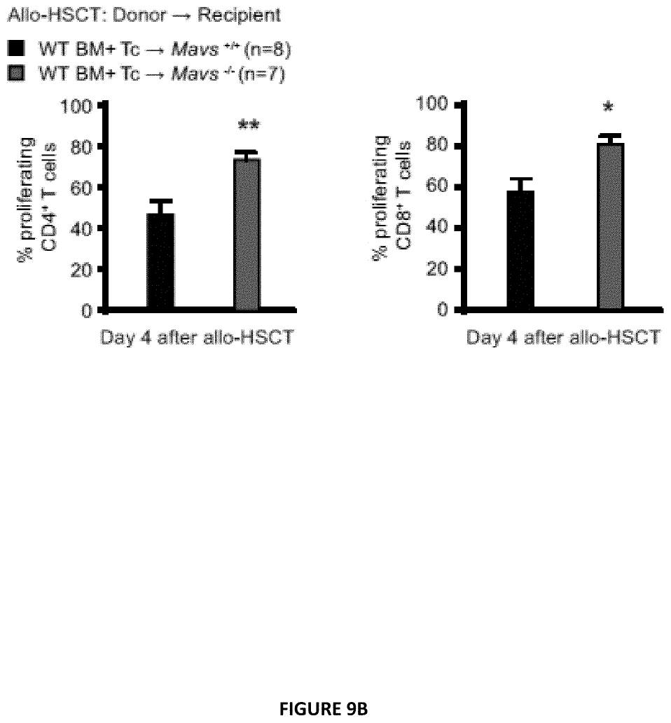

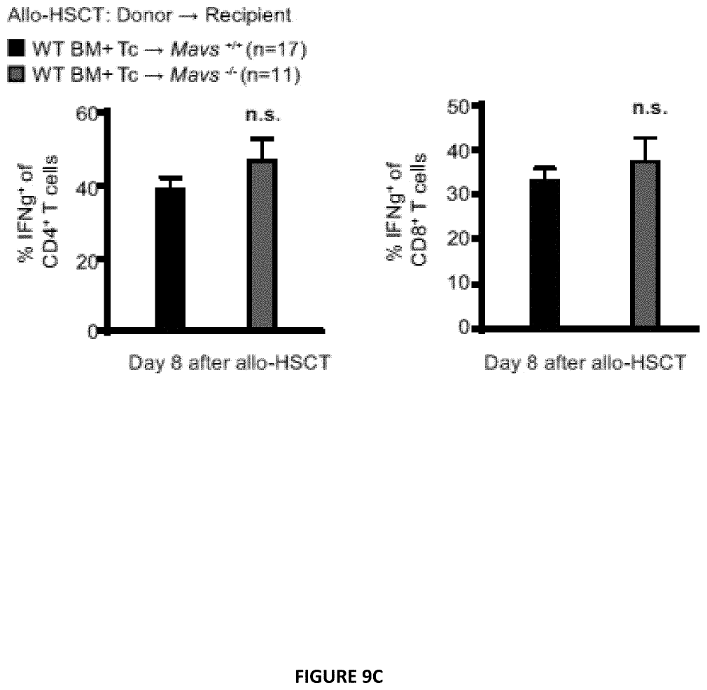

Given that the RIG-I/MAVS pathway senses bacterial RNA (18), one hypothesis to explain our findings was that there may be mouse strain-specific differences in the intestinal bacterial microbiota. We could not detect differences between the intestinal bacterial composition of cohoused Mavs^{-/-} and Mavs^{+/+} littermates as assessed by 16S rRNA sequencing (FIG. 2A). To define the effects of RIG-I/MAVS deficiency in a compartment-specific manner, we generated bone marrow chimeras with either a MAVS-deficient or MAVS-sufficient hematopoietic system or non-hematopoietic system, respectively. This approach yielded donor chimerism of >99% among intestinal myeloid cells (FIG. 9A). Bone marrow chimeras with MAVS deleted in the non-hematopoietic system (MAVS^{+/+} bone marrow transplanted into Mavs^{-/-} recipients) showed higher mortality after allo-HSCT (FIG. 2B) and more intestinal GVHD pathology in the small intestine (FIG. 2C) compared to wild-type recipients of wild-type bone marrow (MAVS^{+/+} bone marrow transplanted into Mavs^{+/+} recipients) or wild-type recipients with MAVS deleted in the hematopoietic system (MAVS^{-/-} bone marrow transplanted into Mavs^{+/+} recipients). We next analyzed gene expression of integrin beta 6 (ltgb6) in small intestine RNA isolates as an indicator of epithelial cell integrity after damage (19, 20) and of the anti-microbial peptide Reglllγ, which is produced by Paneth cells and protects the inner mucus layer from bacterial colonisation (21). Both ltgb6 and Reglllγ gene expression were reduced in Mavs^{-/-} allo-HSCT recipients compared to Mavs^{+/+} littermates (FIG. 2D). As reduced gut epithelial barrier function may promote allogeneic T cell reactivity, we next analyzed donor-derived CD4 and CD8 T cell expansion and IFN-γ production of Mavs^{+/+} versus Mavs^{-/-} allo-HSCT recipient mice. We observed increased T cell proliferation in the spleen of Mavs^{-/-} recipients early after allo-HSCT (day 4 after transplant), but similar T cell effector function at later time points in the small intestine (day 8 after transplant) (FIG. 9B, C).

### Example 4

### Activation of the RIG-I/MAVS Pathway Protects Mice from Intestinal Tissue Damage after Conditioning Therapy

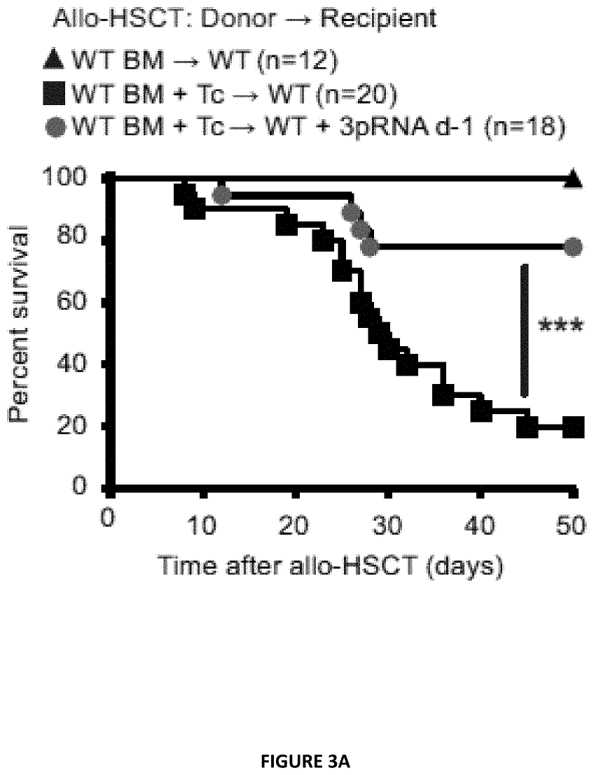

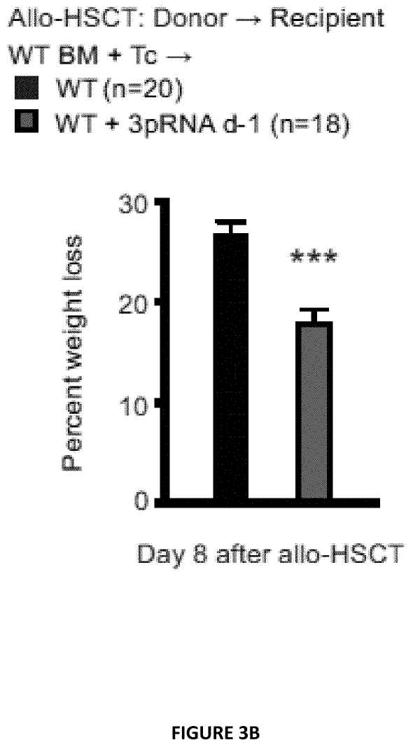

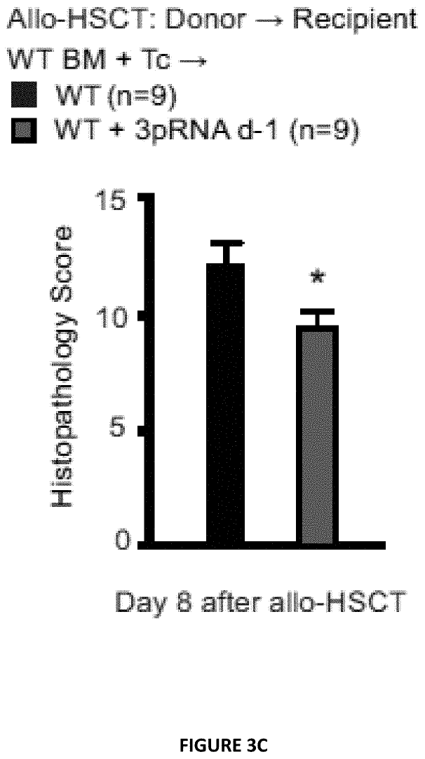

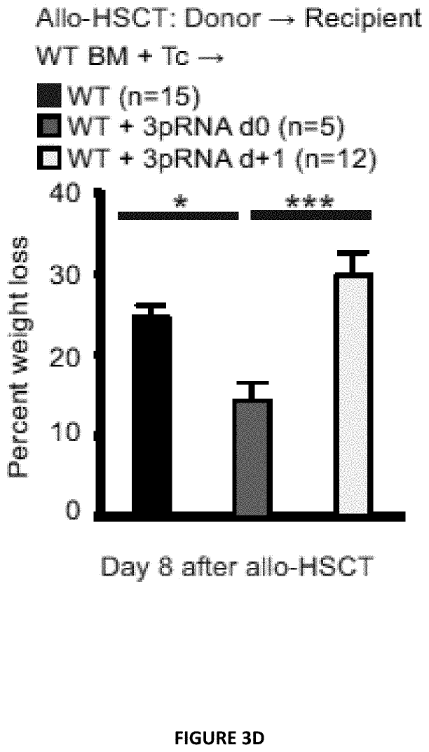

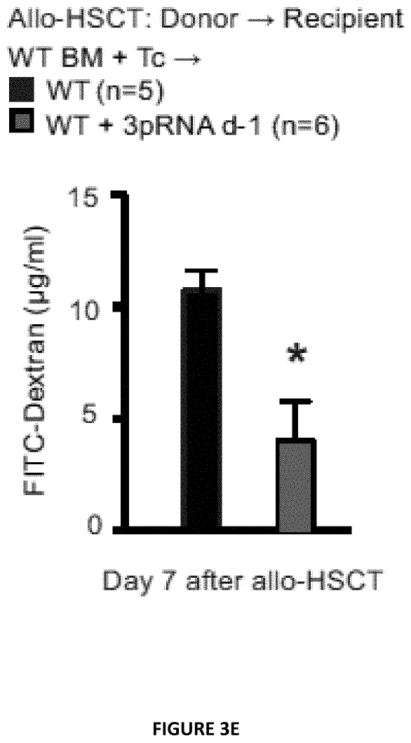

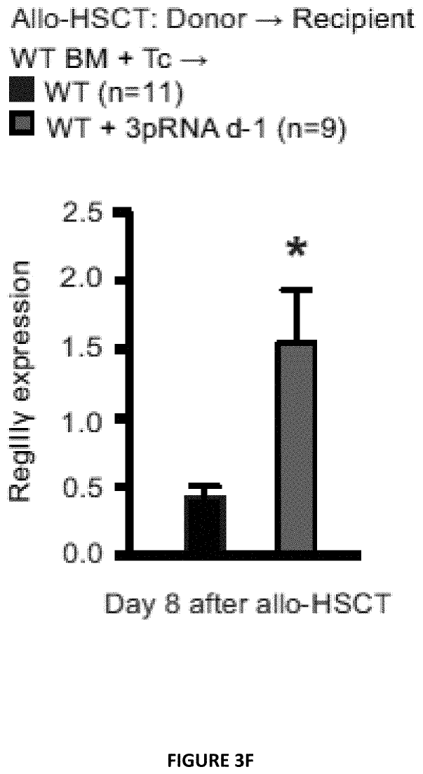

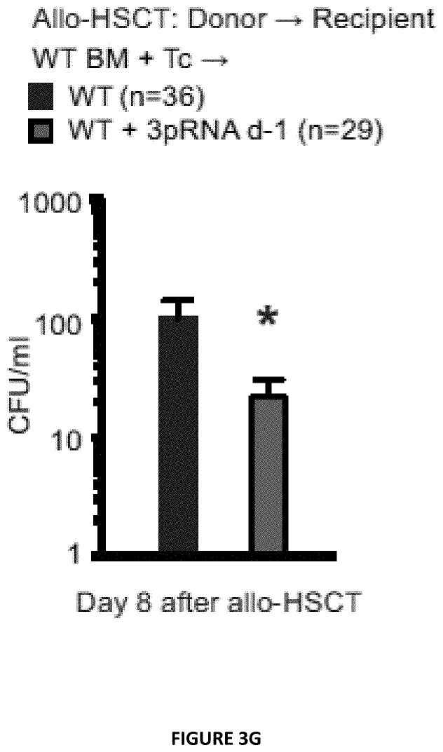

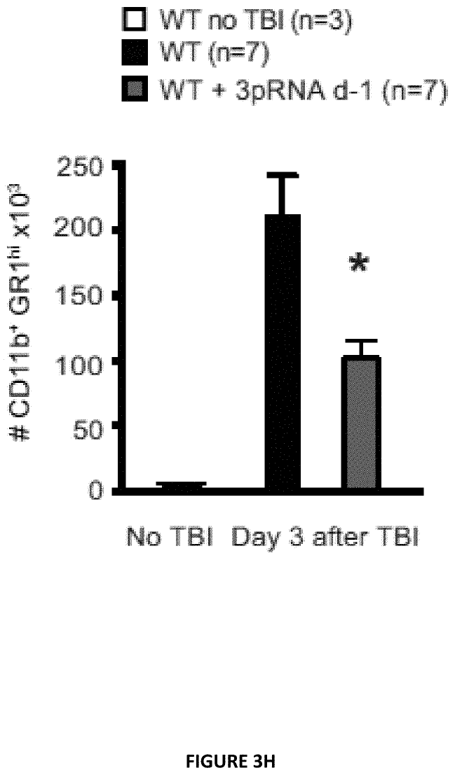

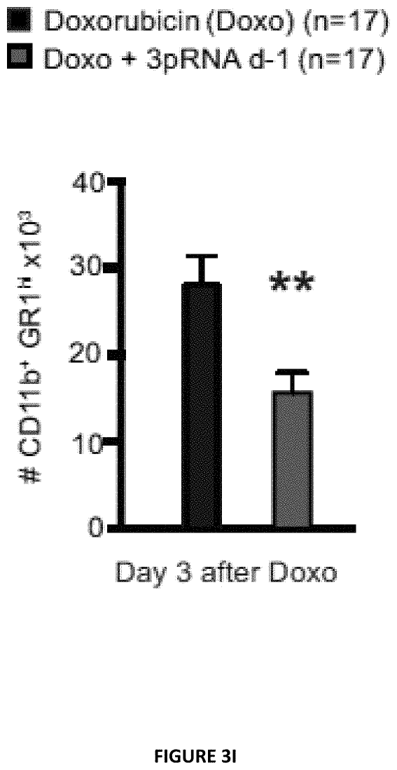

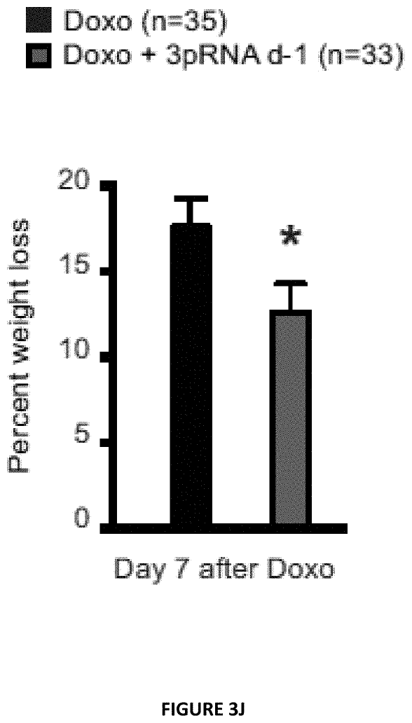

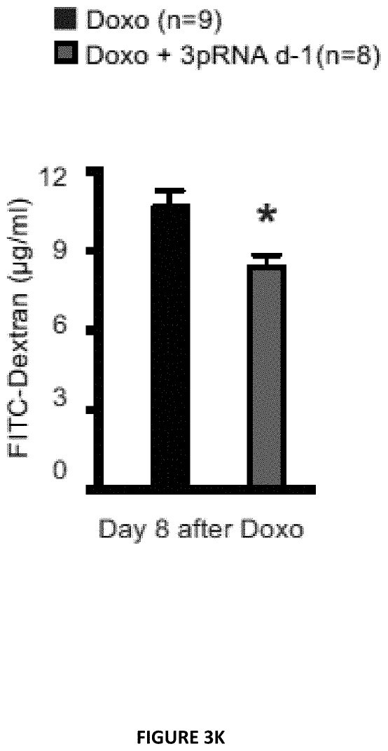

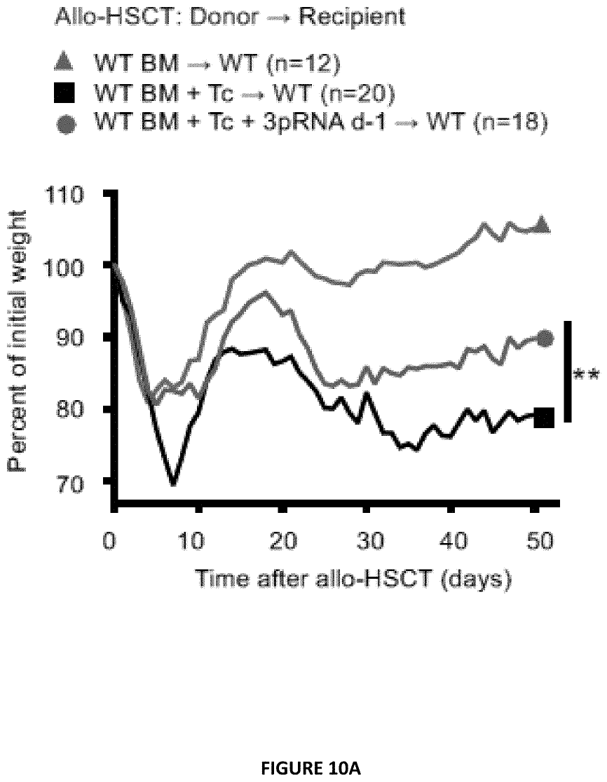

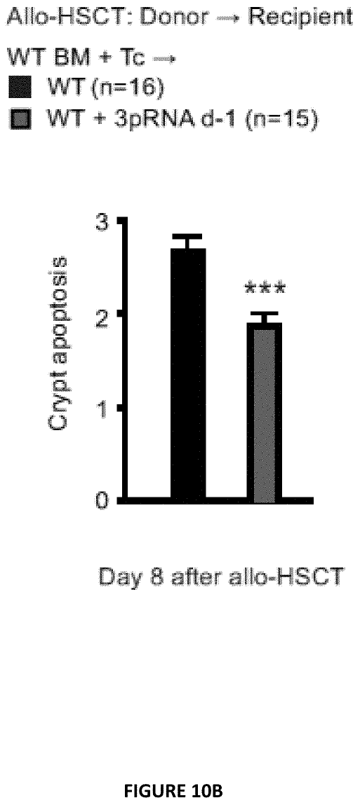

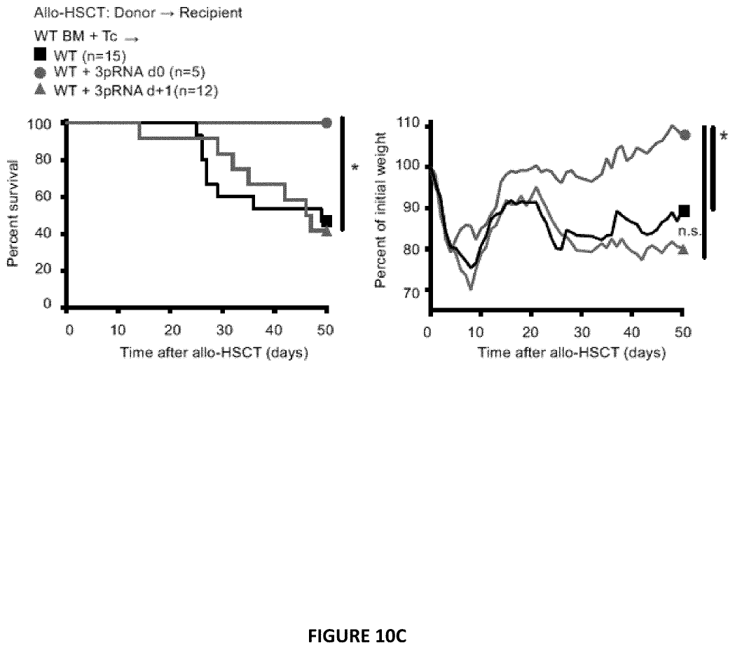

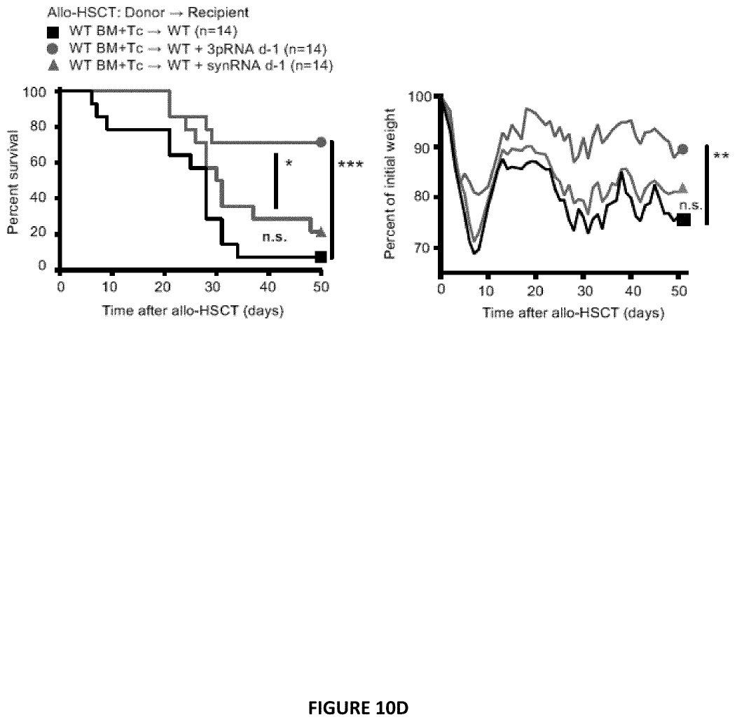

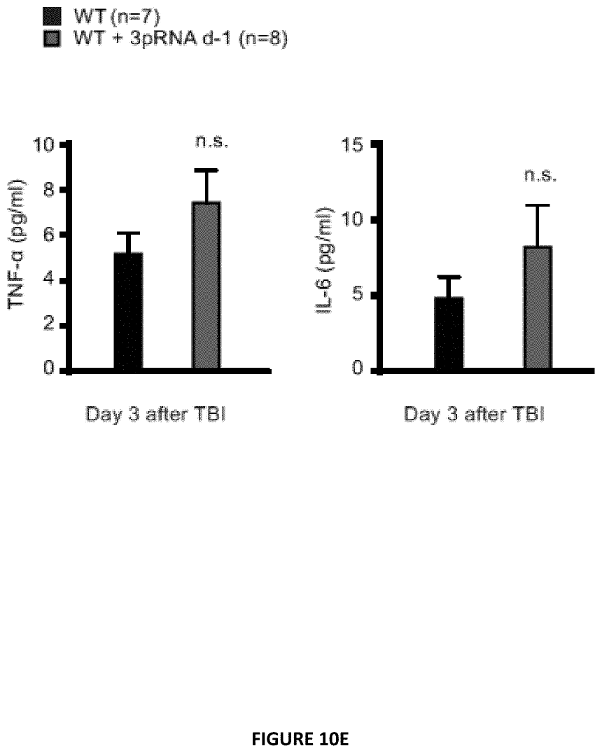

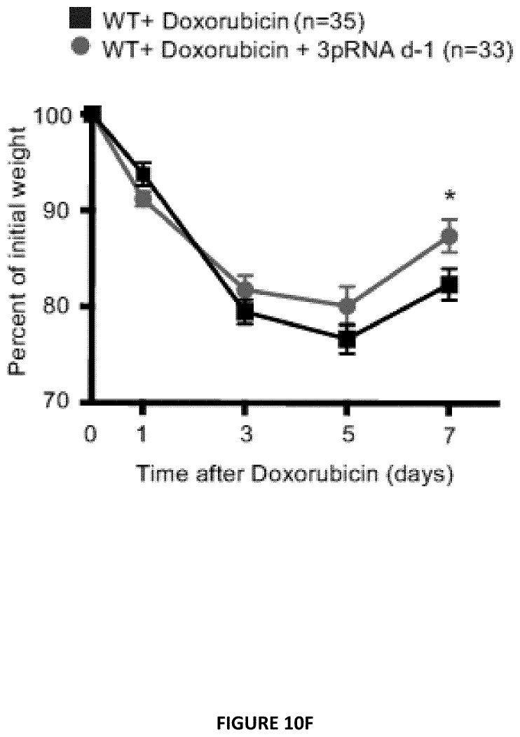

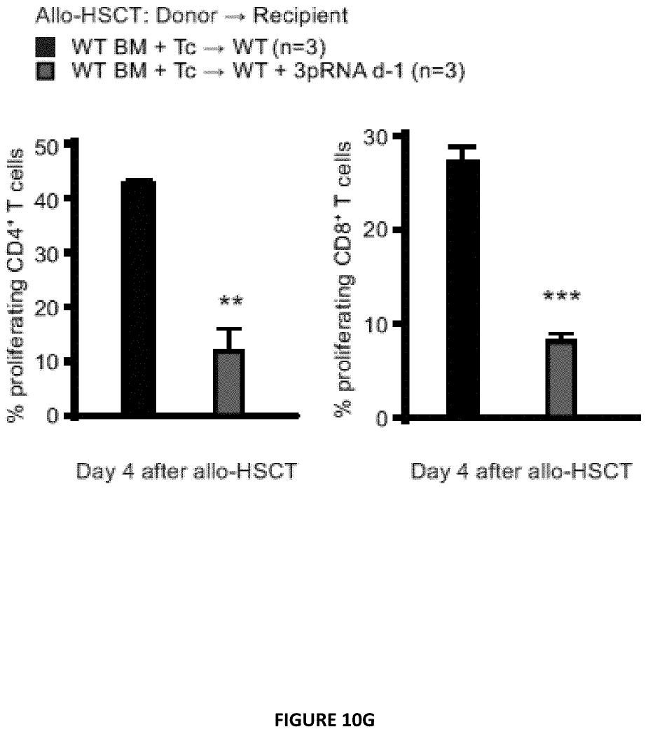

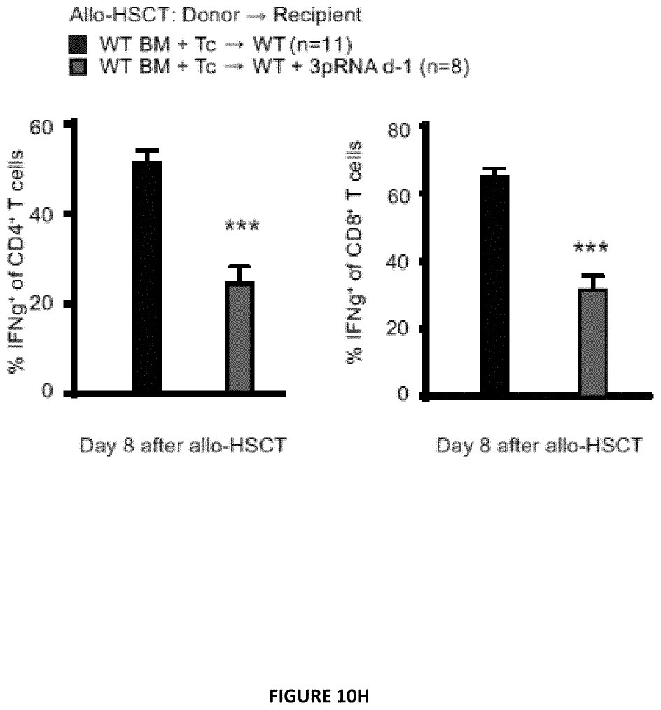

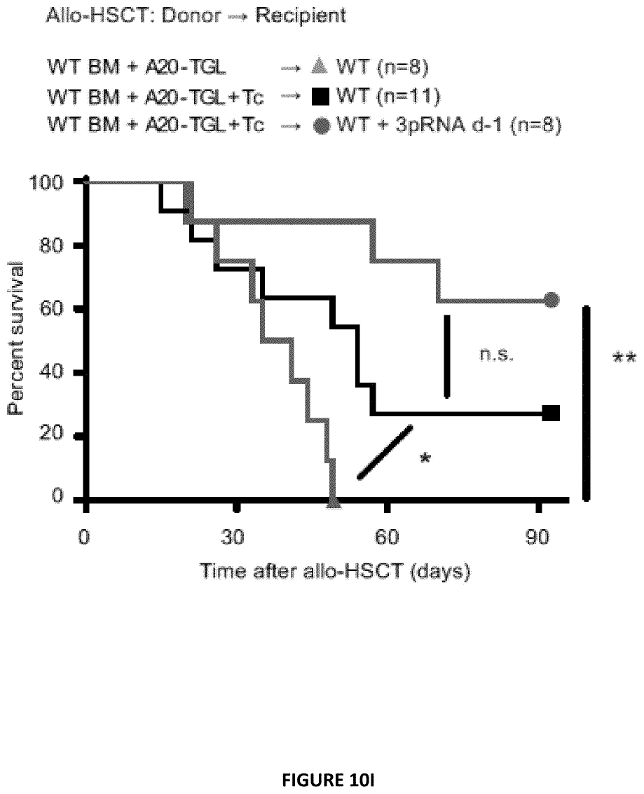

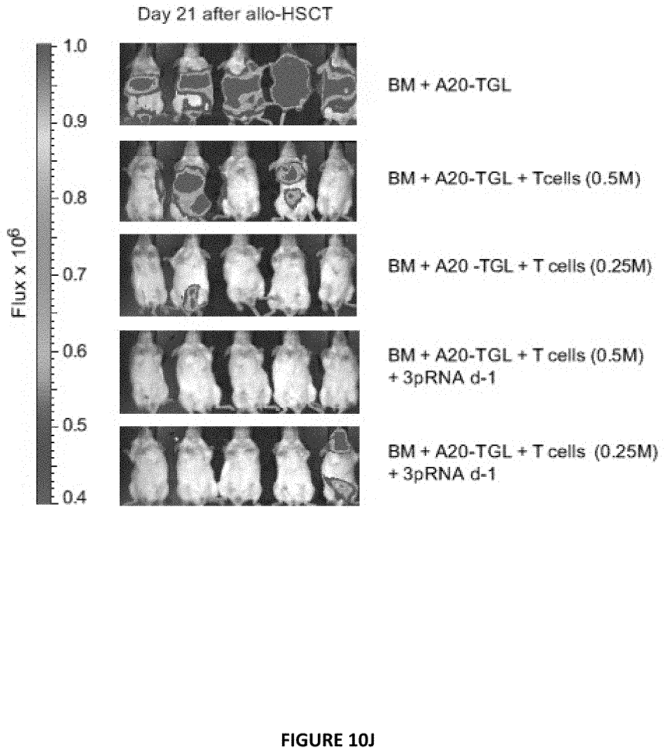

We observed that a single dose of intravenous 3pRNA, a RIG-I agonist, one day before allo-HSCT reduced mortality (FIG. 3A), weight loss (FIG. 3B, and FIG. 10A) and damage to the small intestine compared to control wild-type recipients who did not receive 3pRNA (FIG. 3C, FIG. 10B). It was critical that RIG-I agonists were administered before (day −1) or at the same time as allo-HSCT (day 0), given that administration after allo-HSCT (day +1) failed to achieve a benefit (FIG. 3D, FIG. 10C). Non-triphosphorylated RNA that does not activate RIG-I (22) did not reduce weight loss or GVHD survival (FIG. 10D). This suggested that activation of the RIG-I/MAVS pathway protects from intestinal damage. Administration of 3pRNA (day −1) led to decreased gut mucosal permeability as measured by FITC-dextran translocation (FIG. 3E) and to enhanced intestinal expression of Reglllγ in the small intestine after allo-HSCT (FIG. 3F). Similarly, pretreatment with 3pRNA lowered systemic bacteremia after allo-HSCT (FIG. 3G) and reduced neutrophil infiltration after TBI (FIG. 3H), but did not result in altered production of the pro-inflammatory cytokines IL-6 and TNF-α as compared to TBI alone (FIG. 10E). 3pRNA treatment before barrier disrupting chemotherapy with doxorubicin also reduced neutrophil infiltration, and decreased weight loss and translocation of FITC-dextran (FIGS. 31-3K and FIG. 10F). Consistent with the concept that avoiding breaching the epithelial barrier could prevent GVHD, 3pRNA pretreatment reduced allogeneic T cell activation in the spleen and intestine of allo-HSCT recipient wild-type mice (FIG. 10G, 10H). Decreased allogeneic T cell activity and GVHD may be accompanied by a reduction in the beneficial graft-vs-leukemia (GVL) response. However, application of 3pRNA on day −1 before allo-HSCT along with A20-Luc transduced lymphoma cells did not diminish GVL activity against the latter compared to control allo-HSCT recipient wild-type mice who did not receive 3pRNA (FIGS. 101, 10J).

### Example 5

### RIG-I-Induced Type I IFN Signaling Mediates Intestinal Tissue Protection and Prevents GVHD in Mice

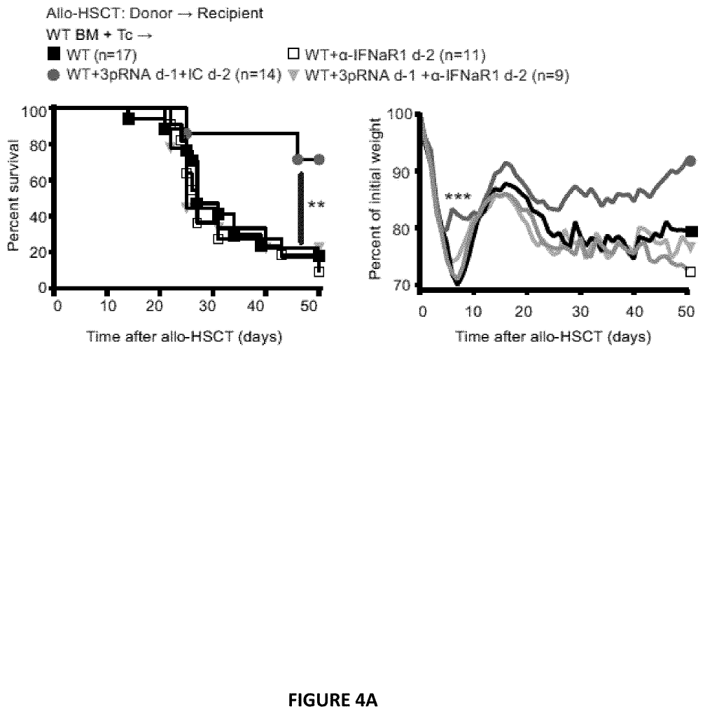

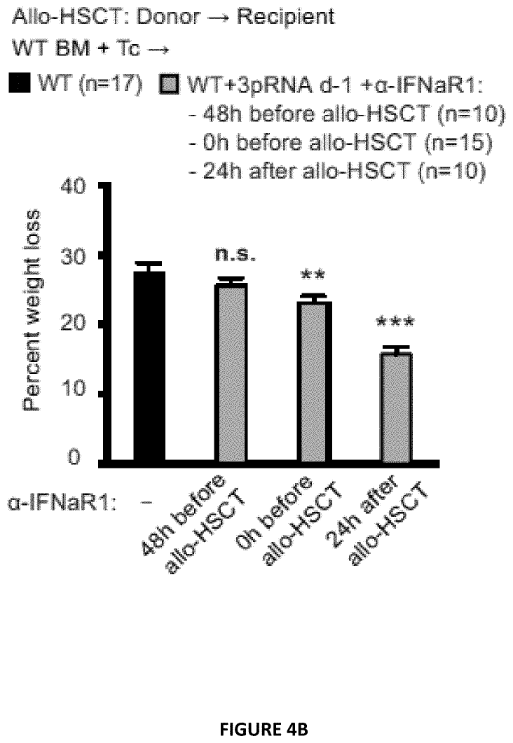

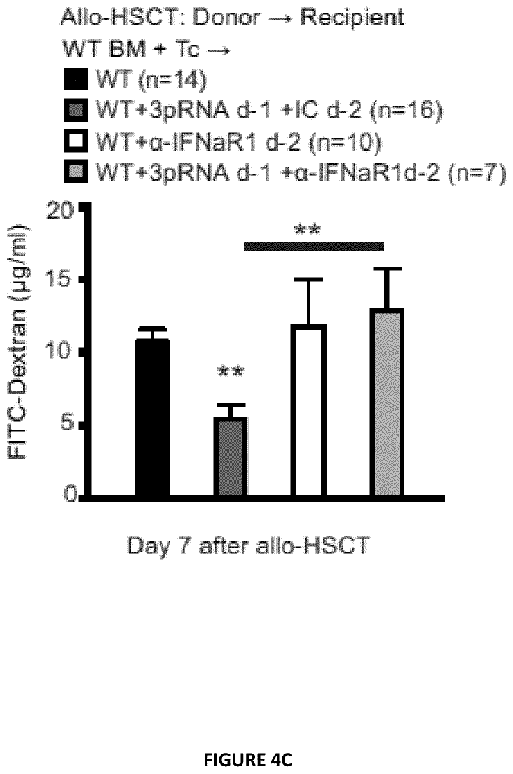

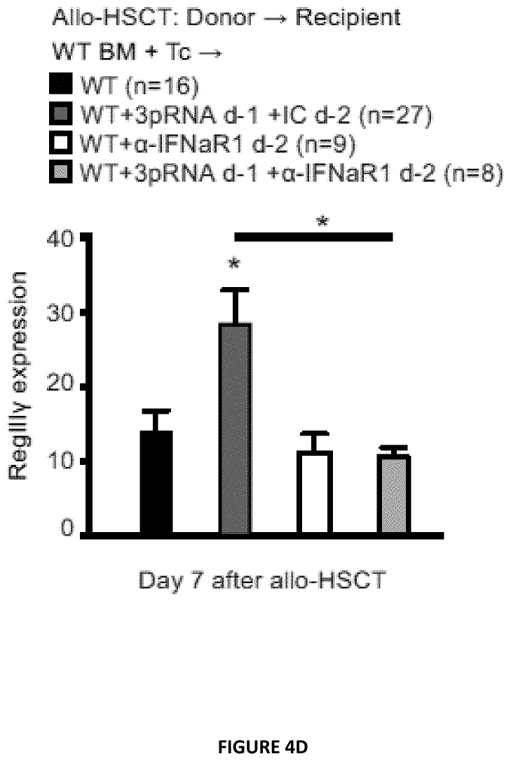

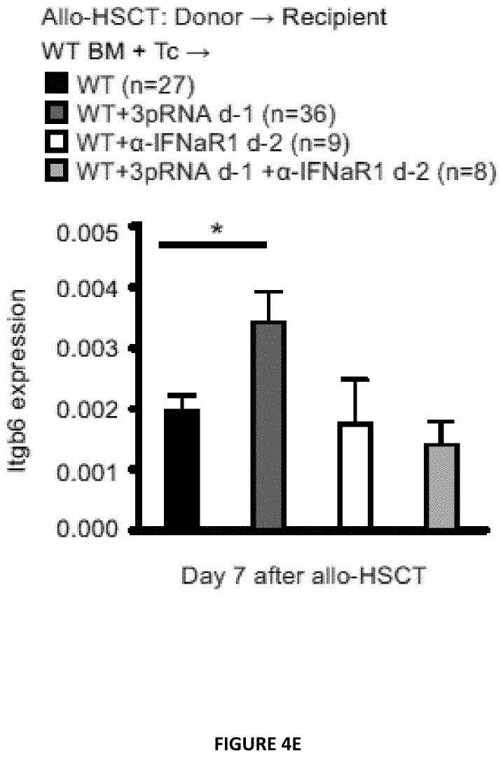

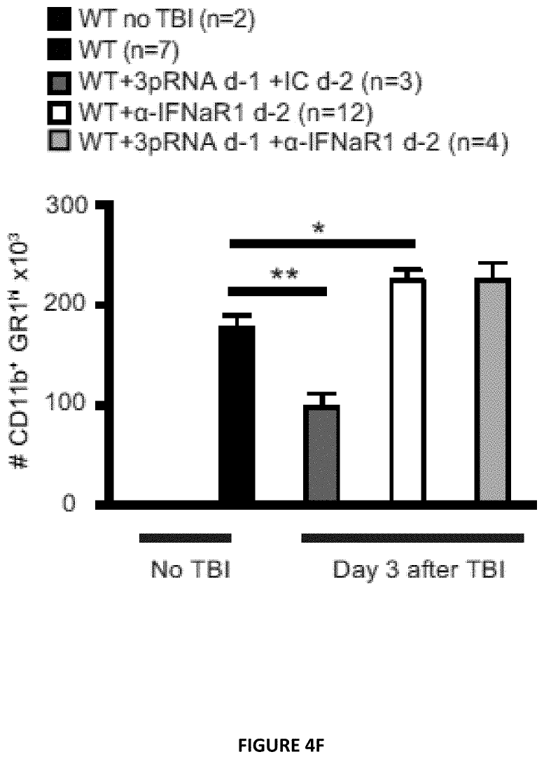

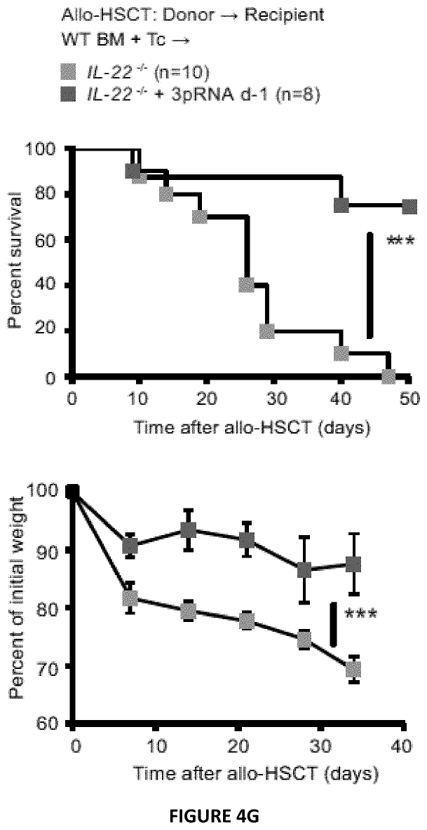

We next analyzed the role of IFN-I in intestinal tissue protection and prevention of GVHD. Systemic application of 3pRNA led to a rapid increase in IFN-α and IFN-β in the serum (FIG. 11A), enhanced IFN-β luciferase reporter activity in the intestine of IFN-β luciferase reporter mice (22) (FIG. 11B) and increased expression of IFN-induced genes including RIG-I (Ddx58) and Mx1 in intestinal epithelial cells isolated from the small intestine (FIG. 11C). We then performed RNA sequencing with tissue samples from the small intestine of wild-type mice that received TBI prior to 3pRNA treatment and antibody-mediated blockade of IFNAR (interferon-α/β receptor) (FIG. 11D). Application of 3pRNA before TBI resulted in increased IFN-inducible genes in the small intestine. Blockade of IFNAR signaling abrogated 3pRNA-mediated upregulation of IFN-induced genes, demonstrating that RIG-I-induced gene-regulation depends on IFN-I. Upon temporary blockade of IFNAR signaling directly before 3pRNA treatment, RIG-I agonists failed to improve overall mouse survival and early weight loss after allo-HSCT (FIG. 4A). This indicated that RIG-I agonists required IFN-I signaling to be protective. In line with our findings with 3pRNA (FIGS. 3B, 3D, FIG. 10C), the induction of IFN-I was only effective before TBI-induced damage. Blocking IFNAR 48 h before tissue damage abrogated the effects of 3pRNA, whereas blockade of IFNAR 24 h after damage did not (FIG. 4B and FIG. 11E). Consistent with these results, blockade of IFN-I signaling two days before TBI-induced damage abrogated the 3pRNA-induced increase in barrier function (FIG. 4C) and the increase in Reglllγ and ltgb6 expression during GVHD (FIGS. 4D, 4E), and reversed the inhibition of neutrophil influx into the mucosa of the small intestine after TBI-induced intestinal damage in wild-type mice (FIG. 4F). Notably, recipient-derived IL-22 has been shown to protect against tissue damage due to conditioning therapy and GVHD (23, 24) by protecting intestinal epithelial integrity. However, 3pRNA was effective in reducing GVHD and weight loss in allo-HSCT recipients (FIG. 4G). This suggested that IFN-I, but not IL-22, may be the mediator of RIG-I-induced protection.

### Example 6

RIG-I-Induced Type I IFN Signaling in Non-Hematopoietic Cells Promotes Proliferation of the Intestinal Stem Cell Compartment

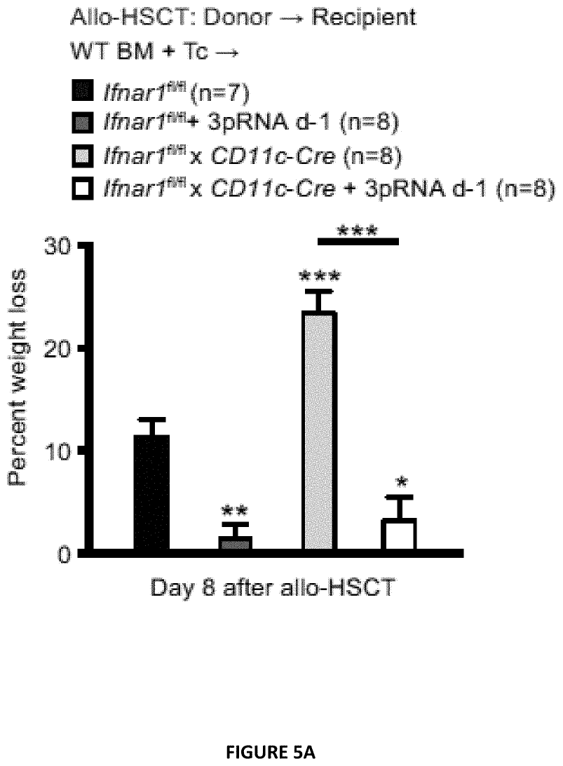

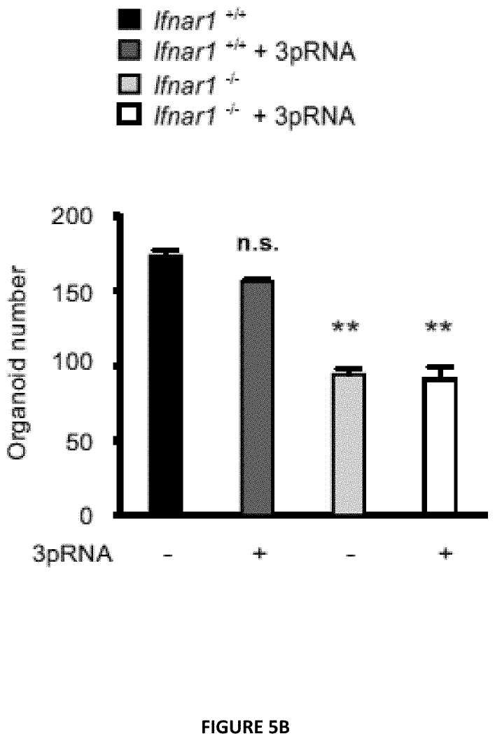

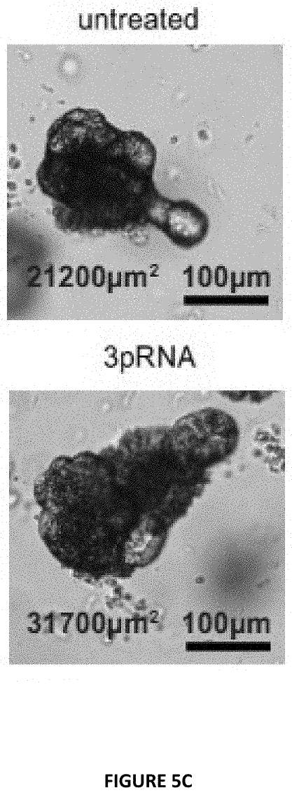

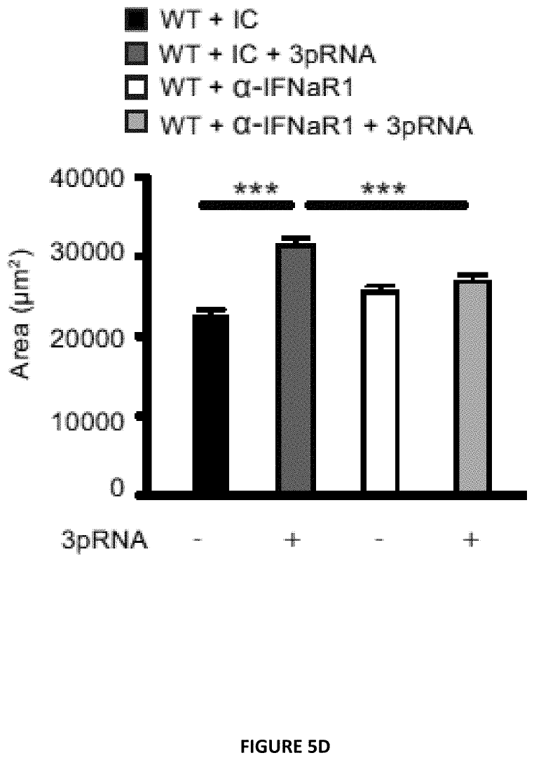

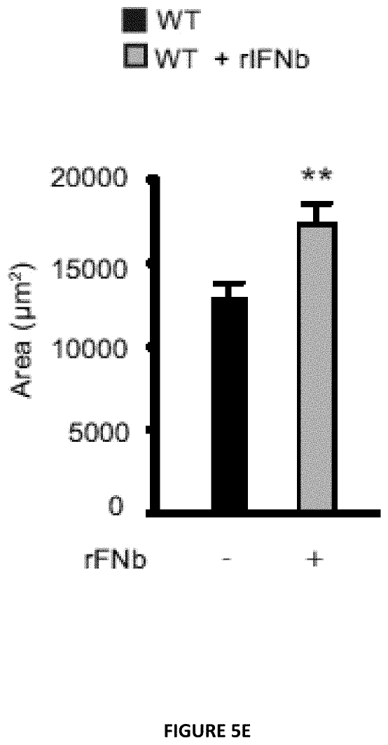

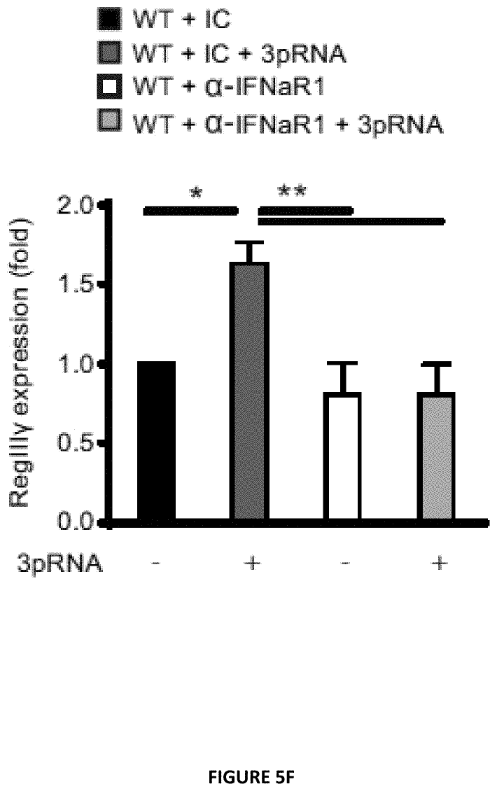

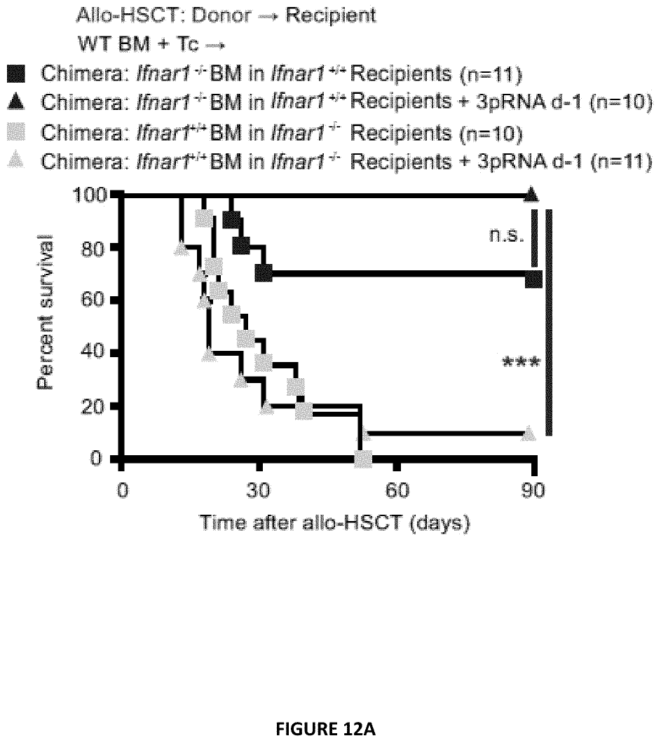

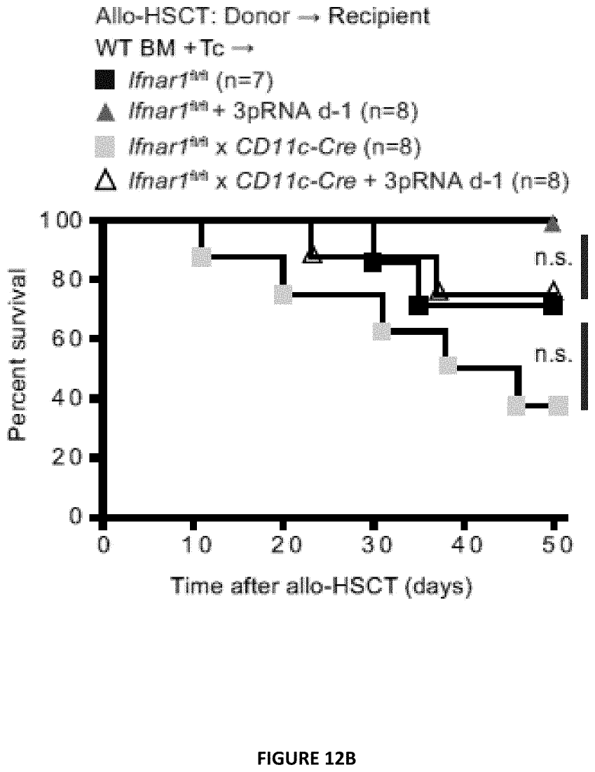

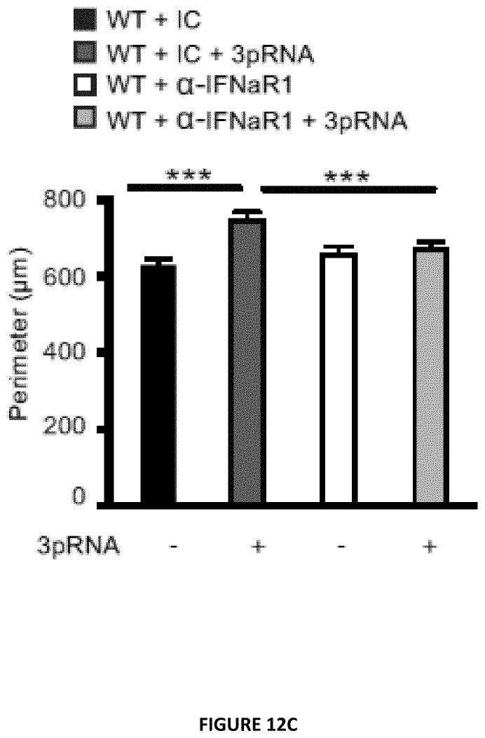

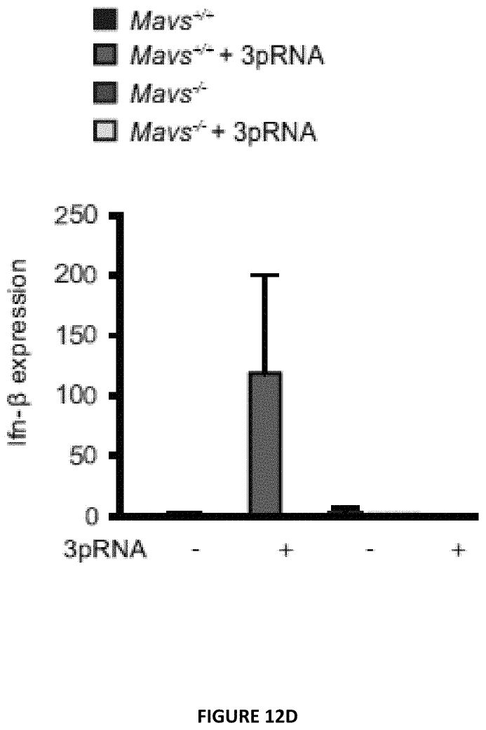

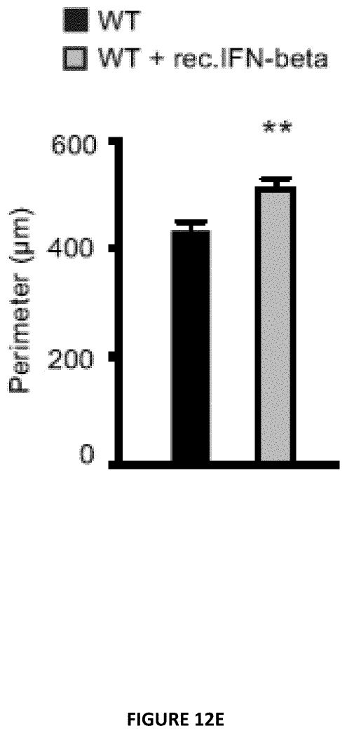

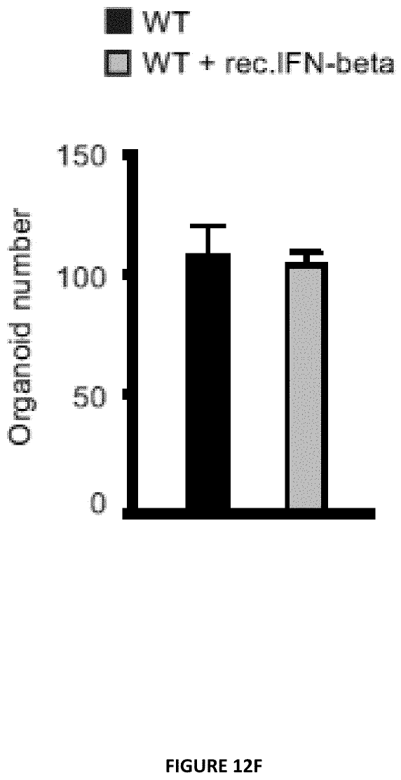

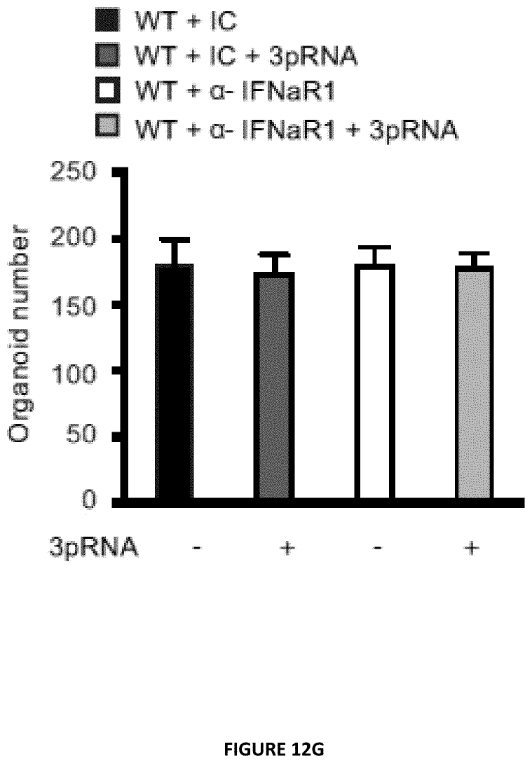

The observed increase in gut barrier function and increased IFN-β production in the intestine after 3pRNA administration (FIG. 11B) suggested a prominent role for the non-hematopoietic system including intestinal epithelial cells as IFN-I targets in vivo. We therefore generated bone marrow chimera with either IFNAR1 (Interferon-alpha/beta receptor alpha chain)-deficient hematopoietic or IFNAR1-deficient non-hematopoietic systems as allo-HSCT recipients for GVHD experiments with or without prior 3pRNA administration. Mice with IFNAR1 deficiency in the non-hematopoietic system developed more severe GVHD than did those with a hematopoietic system-specific deficiency (FIG. 12A). There was a non-statistically significant trend towards improved survival after RIG-I agonist treatment in allo-HCST recipients with an IFNAR1-deficient hematopoietic system, but not in allo-HCST recipients with an IFNAR1-deficient non-hematopoietic system (FIG. 12A). Although these data may suggest a prominent role for IFNAR1, in the non-hematopoietic system, the origin and target of IFNs produced in vivo remain unclear. DCs are a main hematopoietic target population of IFN-I activity in vivo (25), which prompted us to analyze the effects of 3pRNA on the course of GVHD in mice in which CD11c⁺ DCs did not express IFNAR1. We found more early weight loss during GVHD in CD11cCre Ifnar1^{fl/fl} mice, compared to cohoused Ifnar1^{fl/fl} mice (FIG. 5A), whereas overall survival was not significantly different (FIG. 12B). Yet, 3pRNA-mediated prevention of early weight loss was unchanged (FIG. 5A), confirming a predominant role of the non-hematopoietic system in mediating the effects of IFN-I. We thus postulated that prophylactic RIG-I-triggered protection from tissue injury could be mediated by IFN signaling in intestinal epithelial cells. To assess the direct impact of RIG-I signaling and IFN-I on intestinal epithelial cells, we used an ex vivo organoid culture system of mouse primary small intestine crypts (26). Each of these epithelial “mini-guts” contained a functional intestinal stem cell compartment that consisted of LGR5⁺ intestinal stem cells and supportive niche cells (Paneth cells) (26). Crypts cultured ex vivo grew into organoids with crypt buds that recapitulated the in vivo intestinal organization of crypt-villus structures and central lumen markers (27). Interestingly, fewer epithelial organoids were derived from Ifnar1^{-/-} compared to Ifnar1^{+/+} mice suggesting a crucial role for type I IFN signaling in epithelial regeneration (FIG. 5B). Although ex vivo stimulation with 3pRNA did not increase the number of intestinal organoids (FIG. 5B) it did increase organoid size (FIGS. 5C, 5D, FIG. 12C), suggesting that RIG-I activation stimulated the intestinal stem cell compartment resulting in epithelial tissue regeneration.

### Example 7

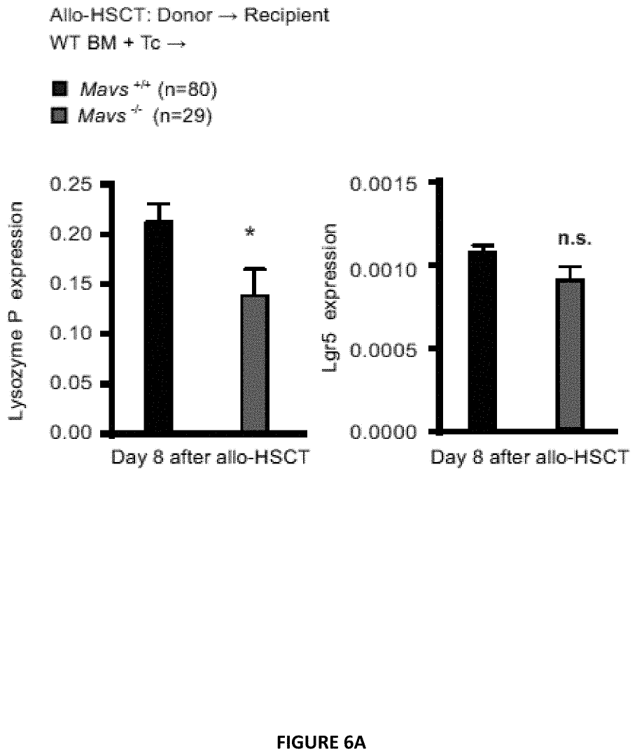

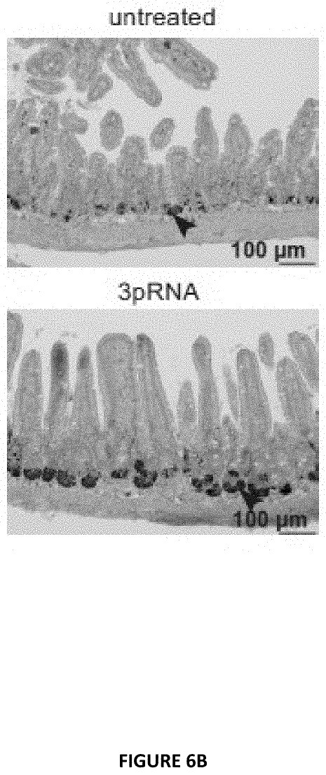

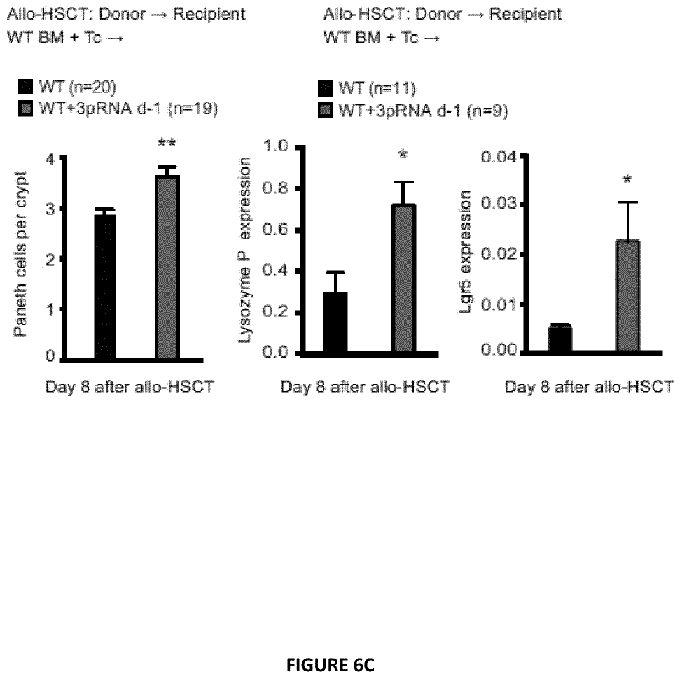

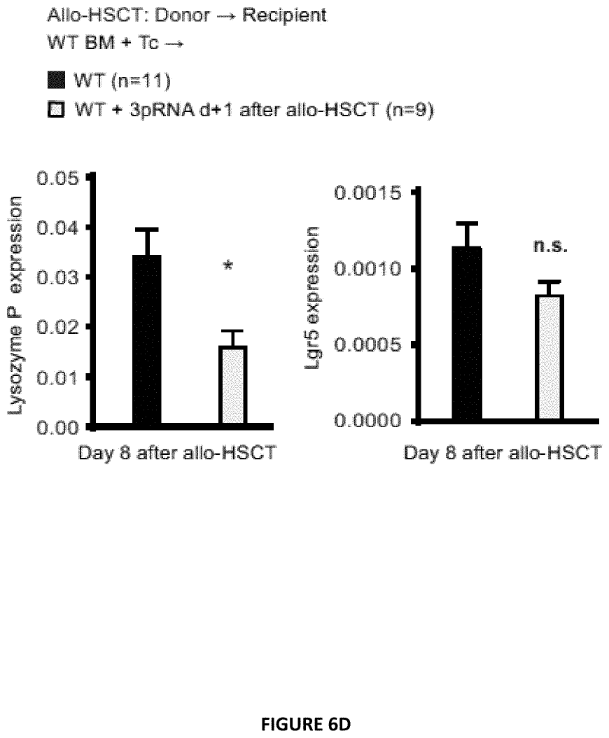

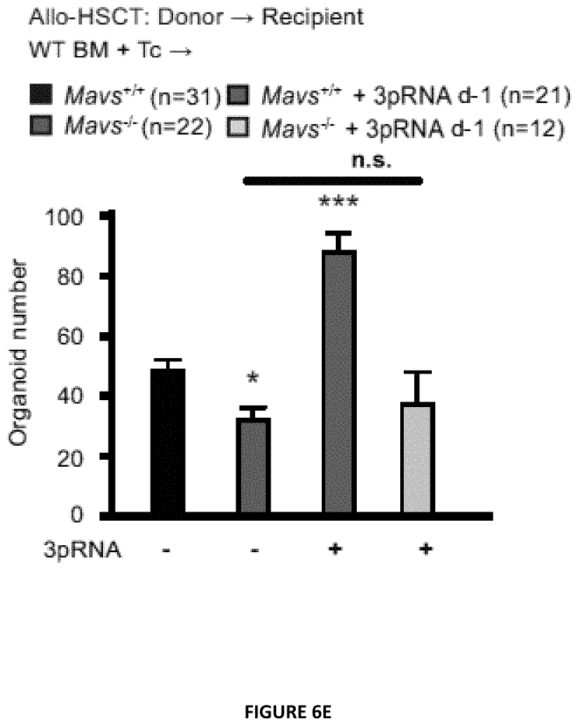

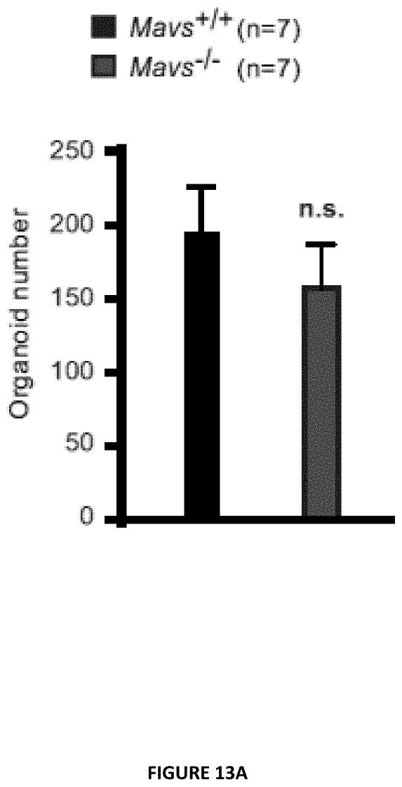

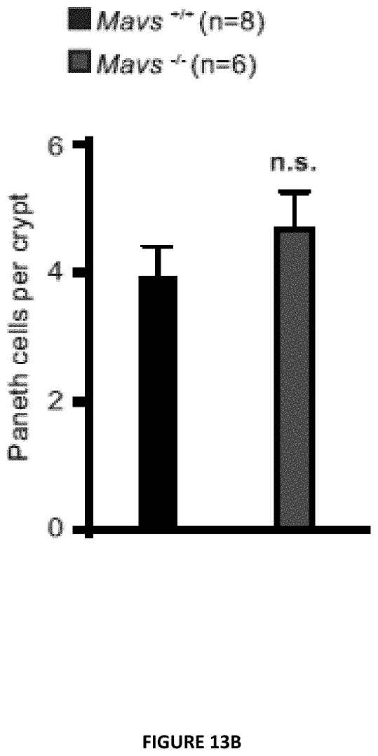

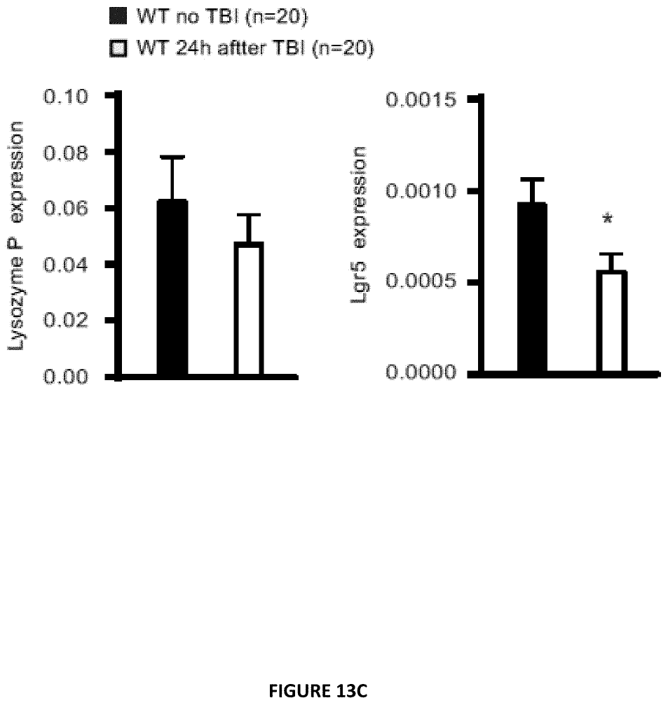

Similar to our in vivo findings demonstrating that 3pRNA-induced augmentation of gut barrier function was mediated by IFNAR signaling (FIGS. 4C-4F), organoid growth after ex vivo 3pRNA stimulation was dependent on IFN-I produced via MAVS (FIG. 12D). IFNAR blockade abrogated 3pRNA-mediated increase in organoid size (FIG. 5D, FIG. 12C), whereas addition of recombinant IFN-β increased organoid size (FIG. 5E, FIG. 12E). Neither recombinant IFN-β nor IFNAR blockade could influence the number of organoids (FIGS. 12F, 12G). Yet, 3pRNA stimulation of organoid-forming crypts ex vivo also induced IFN-I-dependent Reglllγ expression (FIG. 5F), consistent with our in vivo findings during GVHD (FIG. 4D). Unlike with IFNAR^{-/-} organoids, we could not detect an inherent difference in organoid formation between small intestine crypts isolated from Mavs^{+/+} or Mavs^{-/-} littermates (FIG. 13A). Furthermore, no differences in Paneth cell numbers between Mavs^{+/+} and Mavs^{-/-} littermates could be detected (FIG. 13B), suggesting that MAVS signaling in the intestine may not be required for mediating gut homeostasis in the steady-state but is required for the induction of epithelial regeneration after tissue damage. Indeed, we found that the Paneth cell derived antimicrobial peptide Lysozyme P and the intestinal stem cell marker Lgr5 were both reduced in Mavs^{-/-} mice compared to Mavs^{+/+} littermates after allo-HSCT (FIG. 6A). We found elevated numbers of Paneth cells in allo-HSCT recipients pretreated with 3pRNA on day −1 (FIG. 6B) and elevated gene expression of Lysozyme P and the intestinal stem cell marker Lgr5 (FIG. 6C). Congruent with a lack of benefit for GVHD development, we found that delaying 3pRNA treatment until day +1 after allo-HSCT did not protect intestinal stem cells and Paneth cells. Indeed, Lysozyme P and Lgr5 expression was reduced in mice that received 3pRNA on day +1 after allo-HSCT (FIG. 6D). We also observed that Lysozyme P and Lgr5 expression was reduced in mice 24 hours post-TBI, suggesting that the lack of efficacy of 3pRNA post-allo-HSCT could at least in part be due to fewer target cells in the intestinal epithelium (FIG. 13C).

To determine the impact of endogenous RIG-I/MAVS signaling on intestinal regeneration during ongoing GVHD, we next analyzed the capacity to form intestinal organoids ex vivo in Mavs^{+/+} compared to Mavs^{-/-} allo-HSCT recipients. Strikingly, fewer organoids could be retrieved from Mavs^{-/-} allo-HSCT recipients than from Mavs^{+/+} littermates (FIG. 6E). We also found that in vivo 3pRNA treatment prior (day −1) to allo-HSCT induced more organoid growth compared to untreated wild-type recipients. This effect was absent in Mavs^{-/-} allo-HSCT recipients (FIG. 6E), demonstrating that 3pRNA engages the RIG-I/MAVS pathway in vivo to exert its protective function.

### Example 8

### STING Signaling Protects Allo-HSCT Recipients from GVHD and Regulates Intestinal Organoid Growth

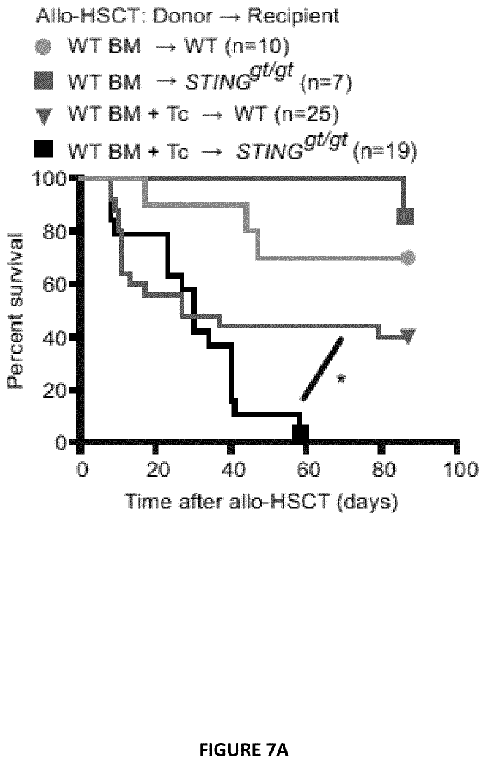

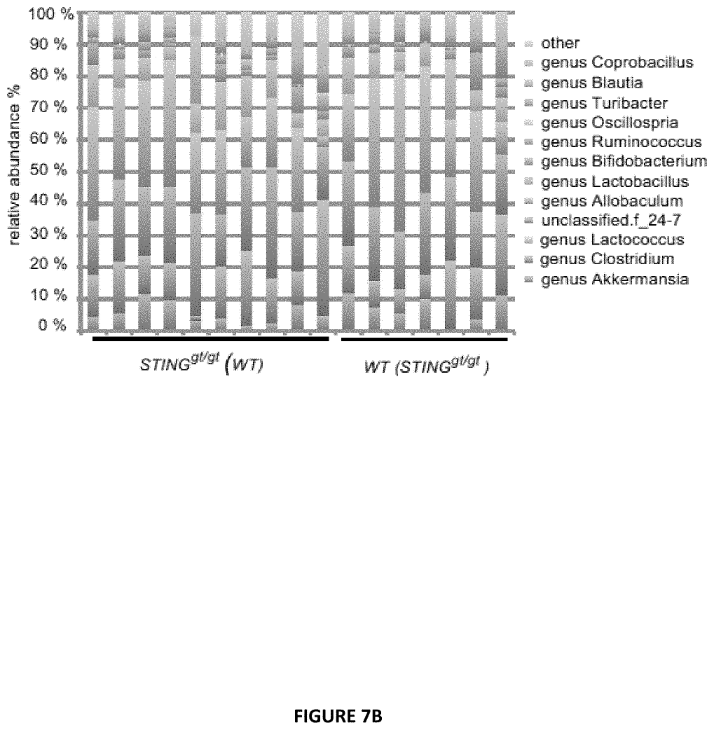

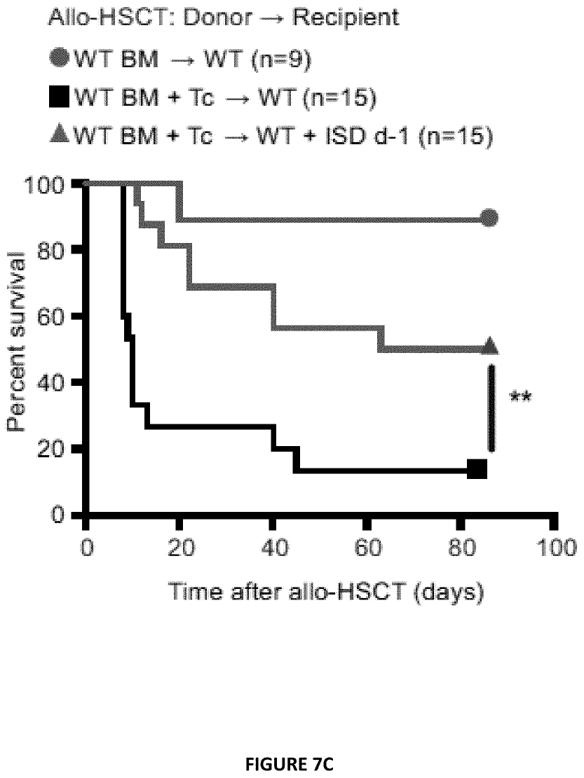

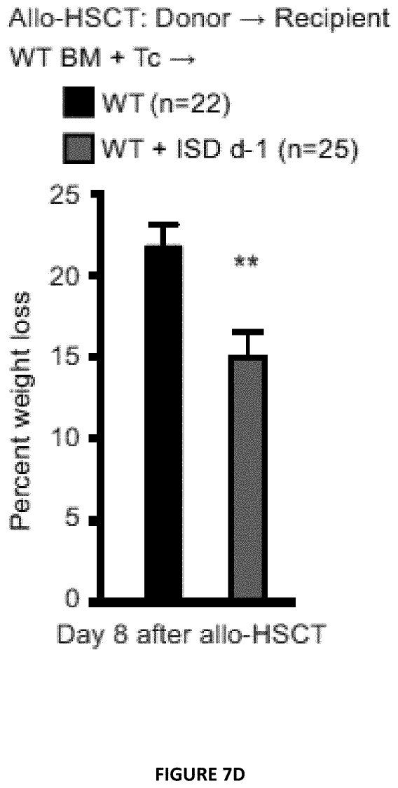

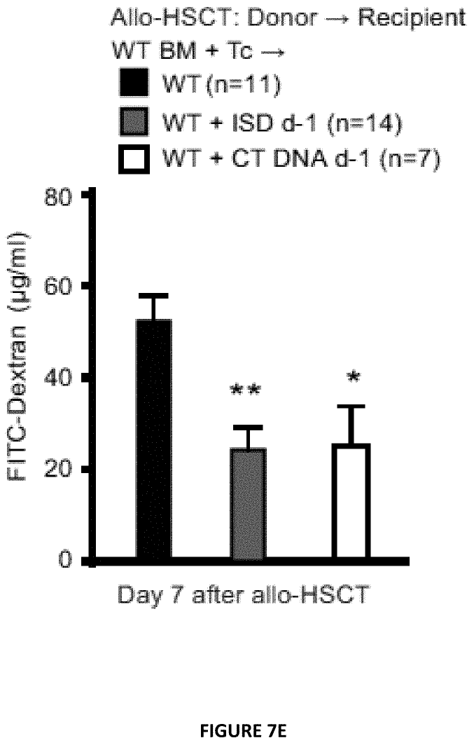

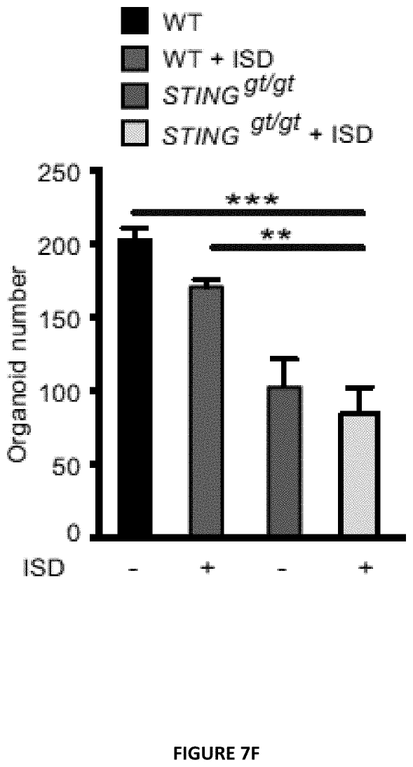

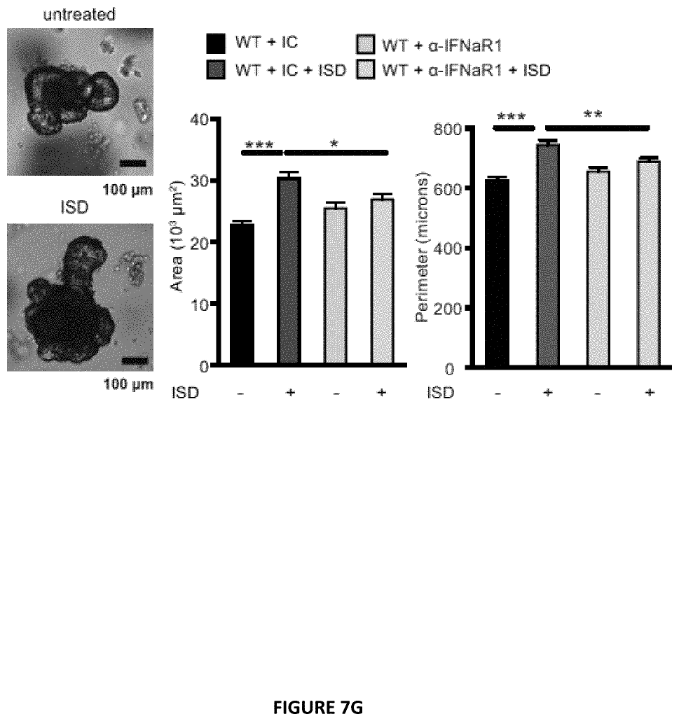

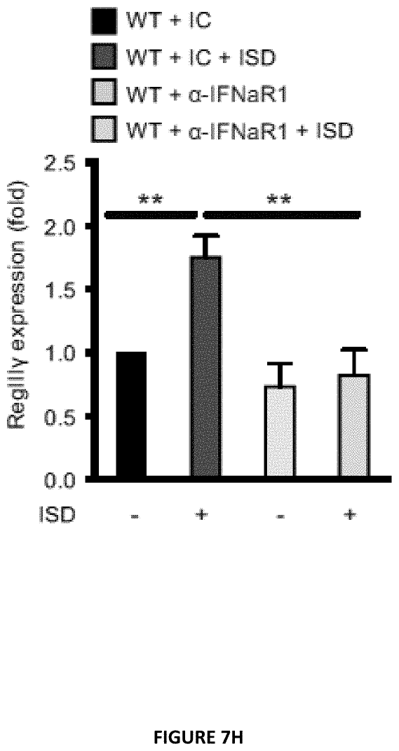

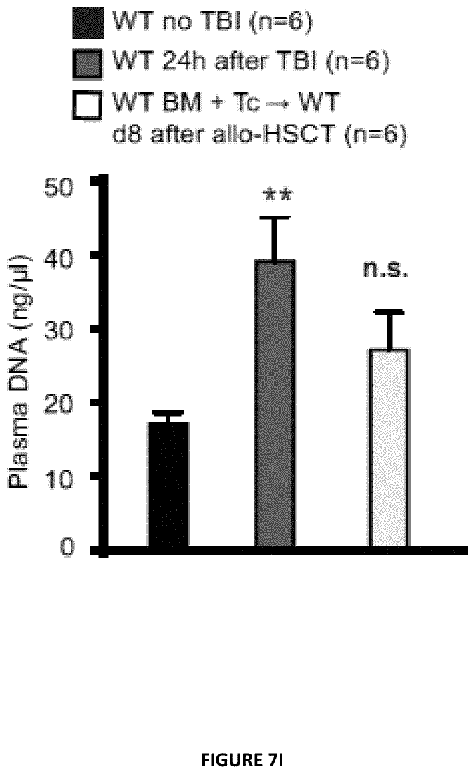

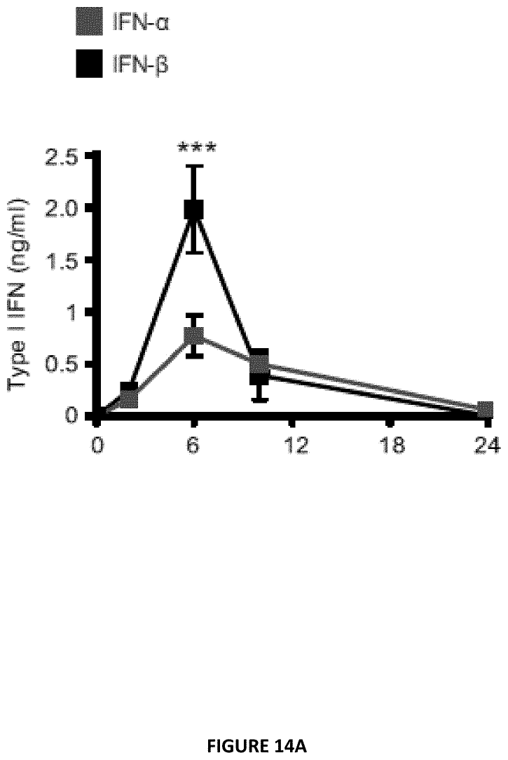

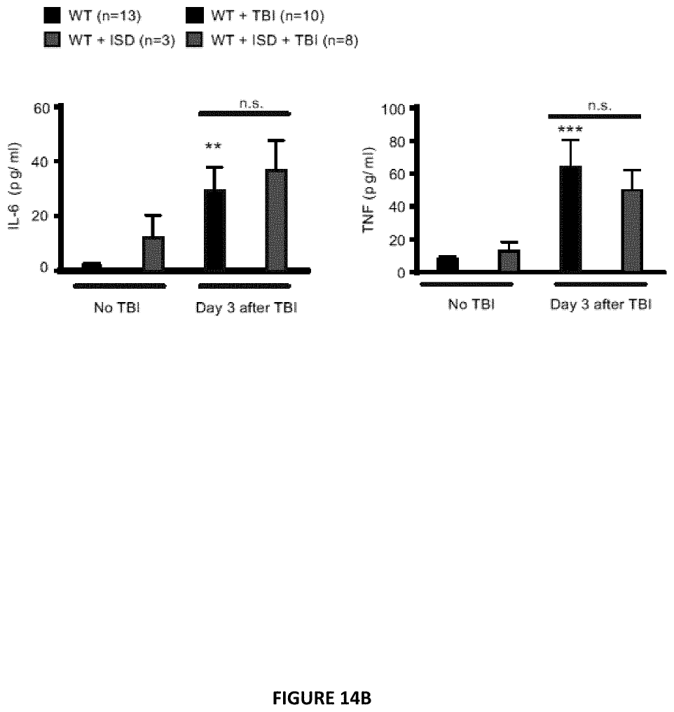

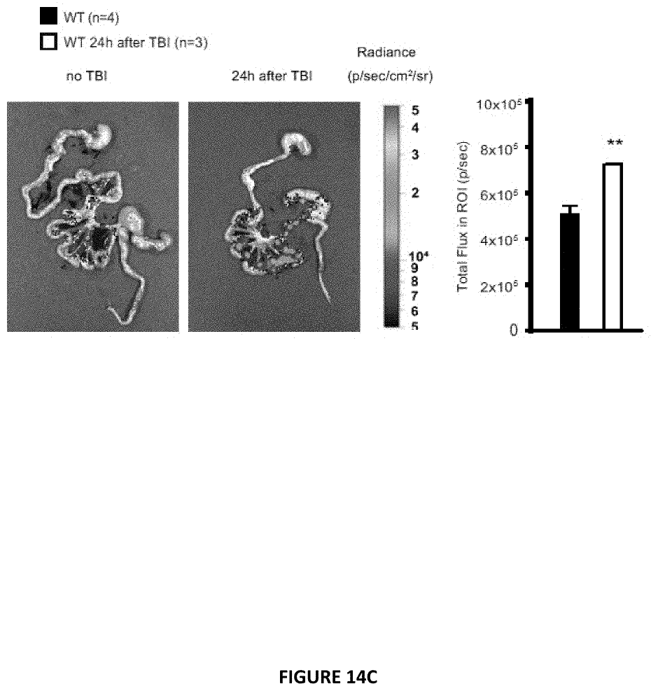

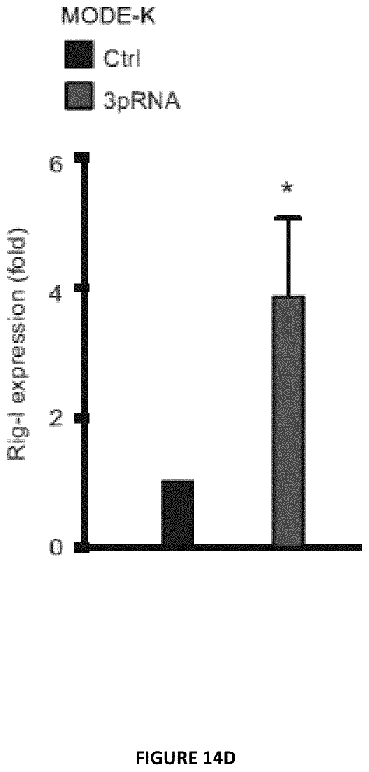

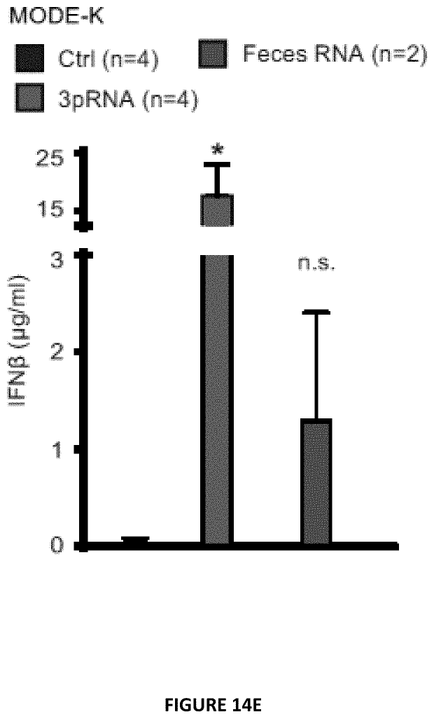

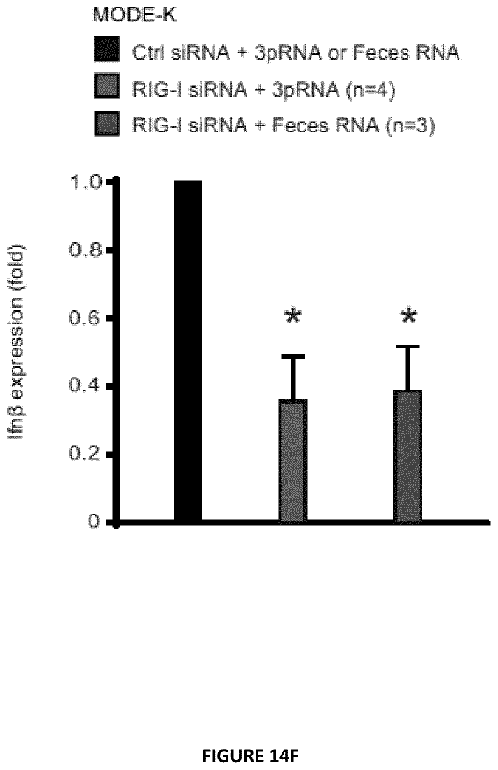

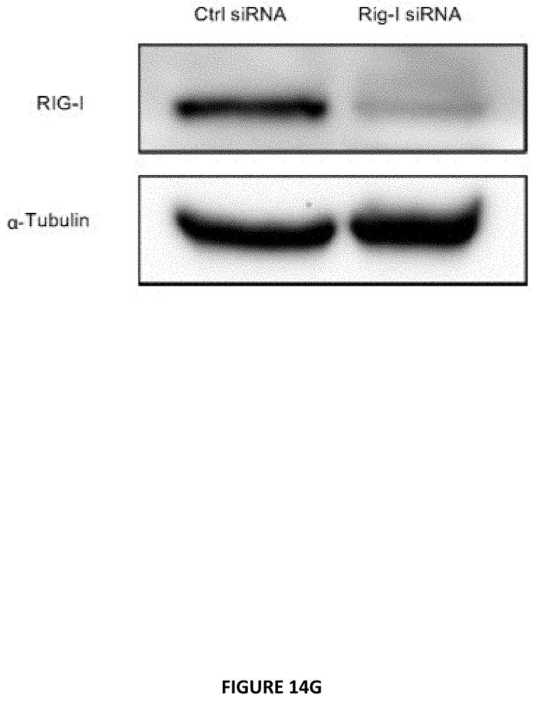

Considering the protective role of RIG-I/MAVS against genotoxic tissue damage, we wondered if other IFN-I inducing cytosolic nucleic acid sensors would have similar effects. We therefore transplanted STING Goldenticket (Sting^{gt/gt}) mice with allogeneic bone marrow and T cells. Similar to our observation in Mavs^{-/-} mice, we found that Sting^{gt/gt} allo-HSCT recipients showed increased mortality compared to cohoused wild-type mice (FIG. 7A), whereas the intestinal bacterial composition was comparable between cohoused wild-type and Sting^{gt/gt} mice (FIG. 7B). In parallel with our 3pRNA results, we found that allo-HSCT recipients treated with interferon stimulatory DNA (ISD) on day −1 showed less mortality (FIG. 7C) and weight loss (FIG. 7D). Interferon stimulatory DNA administration induced production of IFN-α and IFN-β in serum and, like 3pRNA, did not change TBI-induced production of pro-inflammatory cytokines (FIG. 14A, B). Administration of interferon stimulatory DNA also reduced translocation of FITC-dextran across the mouse gut epithelia. This effect could be reproduced after injection of calf thymus DNA, which, in contrast to interferon stimulatory DNA, contained CpG motifs (28) and induced IFN-I via the STING pathway (29) (FIG. 7E). Finally, STING signaling and stimulation of organoids with interferon stimulatory DNA contributed to growth of intestinal organoid cultures and Reglllγ expression in an IFN-I dependent manner (FIGS. 7F-7H).

Given that pre-transplant conditioning by either TBI or chemotherapy leads to accumulation of aberrant self-DNA found in apoptotic bodies, extracellular space and cytosol resulting in IFN-I production (30), we hypothesized that the STING pathway might mediate protection from GVHD via detection of endogenous DNA. Indeed, we observed increased dsDNA in the plasma of mice undergoing TBI compared to untreated mice (FIG. 7I). We next utilized the luciferase-reporter mouse system to analyze IFN-β production in the intestine 24 hours after TBI. In comparison to untreated mice, TBI induced IFN-I signaling in the small intestine (FIG. 14C).

We did not succeed in detecting endogenous RNA in mouse plasma. If endogenous RNA was released into the extracellular space upon damage, we presumed it was rapidly degraded. Given that commensal microbiota including bacteria in the gut could potentially deliver endogenous ligands required for activation of RIG-I (18), we tested if RNA isolated from mouse feces could induce RIG-I-dependent IFN-I signaling in intestinal epithelial cells. Feces-derived RNA induced a RIG-I-dependent IFN-I response in Mode-K cells, a murine intestinal epithelial cell line with morphological and phenotypic characteristics of normal enterocytes (31), arguing that 3pRNA and RNA derived from commensals including viruses, phages or bacteria could potentially induce protective IFN-I signaling via activation of RIG-I (FIGS. 140-14G).

Together, our data suggest that activation of the RIG/MAVS and STING pathways, either through endogenous or applied ligands (ISD, 3pRNA), may be essential for protection of gut epithelial integrity after genotoxic insult and for the prevention of GVHD following allo-HSCT.

Data and Materials Availability:

The RNA sequencing data for this study have been deposited in the database GEO and can be found at GEO GS E87386.

### REFERENCES

1. Z. Abdullah, M. Schlee, S. Roth, M. A. Mraheil, W. Barchet, J. Bottcher, T. Hain, S. Geiger, Y. Hayakawa, J. H. Fritz, F. Civril, K. P. Hopfner, C. Kurts, J. Ruland, G. Hartmann, T. Chakraborty, P. A. Knolle, RIG-I detects infection with live Listeria by sensing secreted bacterial nucleic acids. The EMBO journal 31, 4153-4164 (2012).
2. V. Hornung, J. Ellegast, S. Kim, K. Brzozka, A. Jung, H. Kato, H. Poeck, S. Akira, K. K. Conzelmann, M. Schlee, S. Endres, G. Hartmann, 5′-Triphosphate RNA is the ligand for RIG-I. Science 314, 994-997 (2006).
3. A. Pichlmair, O. Schulz, C. P. Tan, T. I. Naslund, P. Liljestrom, F. Weber, C. Reis e Sousa, RIG-I-mediated antiviral responses to single-stranded RNA bearing 5′-phosphates. Science 314, 997-1001 (2006).
4. G. N. Barber, STING-dependent cytosolic DNA sensing pathways. Trends in immunology 35, 88-93 (2014).
5. H. Poeck, M. Bscheider, O. Gross, K. Finger, S. Roth, M. Rebsamen, N. Hannesschlager, M. Schlee, S. Rothenfusser, W. Barchet, H. Kato, S. Akira, S. Inoue, S. Endres, C. Peschel, G. Hartmann, V. Hornung, J. Ruland, Recognition of RNA virus by RIG-I results in activation of CARD9 and inflammasome signaling for interleukin 1 beta production. Nature immunology 11, 63-69 (2010).
6. M. Yoneyama, K. Onomoto, M. Jogi, T. Akaboshi, T. Fujita, Viral RNA detection by RIG-I-like receptors. Current opinion in immunology 32C, 48-53 (2015).
7. J. Pothlichet, I. Meunier, B. K. Davis, J. P. Ting, E. Skamene, V. von Messling, S. M. Vidal, Type I IFN triggers RIG-I/TLR3/NLRP3-dependent inflammasome activation in influenza A virus infected cells. PLoS pathogens 9, e1003256 (2013).
8. L. Franchi, T. Eigenbrod, R. Munoz-Planillo, U. Ozkurede, Y. G. Kim, A. Chakrabarti, M. Gale, Jr., R. H. Silverman, M. Colonna, S. Akira, G. Nunez, Cytosolic double-stranded RNA activates the NLRP3 inflammasome via MAVS-induced membrane permeabilization and K+ efflux. Journal of immunology 193, 4214-4222 (2014).
9. J. Gregorio, S. Meller, C. Conrad, A. Di Nardo, B. Homey, A. Lauerma, N. Arai, R. L. Gallo, J. Digiovanni, M. Gilliet, Plasmacytoid dendritic cells sense skin injury and promote wound healing through type I interferons. The Journal of experimental medicine 207, 2921-2930 (2010).
10. L. Sun, H. Miyoshi, S. Origanti, T. J. Nice, A. C. Barger, N. A. Manieri, L. A. Fogel, A. R. French, D. Piwnica-Worms, H. Piwnica-Worms, H. W. Virgin, D. J. Lenschow, T. S. Stappenbeck, Type I interferons link viral infection to enhanced epithelial turnover and repair. Cell host & microbe 17, 85-97 (2015).
11. J. Y. Yang, M. S. Kim, E. Kim, J. H. Cheon, Y. S. Lee, Y. Kim, S. H. Lee, S. U. Seo, S. H. Shin, S. S. Choi, B. Kim, S. Y. Chang, H. J. Ko, J. W. Bae, M. N. Kweon, Enteric Viruses Ameliorate Gut Inflammation via Toll-like Receptor 3 and Toll-like Receptor 7-Mediated Interferon-beta Production. Immunity 44, 889-900 (2016).
12. L. W. Peterson, D. Artis, Intestinal epithelial cells: regulators of barrier function and immune homeostasis. Nature reviews. Immunology 14, 141-153 (2014).
13. S. Heidegger, M. R. van den Brink, T. Haas, H. Poeck, The role of pattern-recognition receptors in graft-versus-host disease and graft-versus-leukemia after allogeneic stem cell transplantation. Frontiers in immunology 5, 337 (2014).
14. B. R. Blazar, W. J. Murphy, M. Abedi, Advances in graft-versus-host disease biology and therapy. Nature reviews. Immunology 12, 443-458 (2012).
15. R. R. Jenq, M. R. van den Brink, Allogeneic haematopoietic stem cell transplantation: individualized stem cell and immune therapy of cancer. Nature reviews. Cancer 10, 213-221 (2010).
16. L. Schwab, L. Goroncy, S. Palaniyandi, S. Gautam, A. Triantafyllopoulou, A. Mocsai, W. Reichardt, F. J. Karlsson, S. V. Radhakrishnan, K. Hanke, A. Schmitt-Graeff, M. Freudenberg, F. D. von Loewenich, P. Wolf, F. Leonhardt, N. Baxan, D. Pfeifer, O. Schmah, A. Schonle, S. F. Martin, R. Mertelsmann, J. Duyster, J. Finke, M. Prinz, P. Henneke, H. Hacker, G. C. Hildebrandt, G. Hacker, R. Zeiser, Neutrophil granulocytes recruited upon translocation of intestinal bacteria enhance graft-versus-host disease via tissue damage. Nature medicine 20, 648-654 (2014).
17. S. Viaud, F. Saccheri, G. Mignot, T. Yamazaki, R. Daillere, D. Hannani, D. P. Enot, C. Pfirschke, C. Engblom, M. J. Pittet, A. Schlitzer, F. Ginhoux, L. Apetoh, E. Chachaty, P. L. Woerther, G. Eberl, M. Berard, C. Ecobichon, D. Clermont, C. Bizet, V. Gaboriau-Routhiau, N. Cerf-Bensussan, P. Opolon, N. Yessaad, E. Vivier, B. Ryffel, C. O. Elson, J. Dore, G. Kroemer, P. Lepage, I. G. Boneca, F. Ghiringhelli, L. Zitvogel, The intestinal microbiota modulates the anticancer immune effects of cyclophosphamide. Science 342, 971-976 (2013).
18. X. D. Li, Y. H. Chiu, A. S. Ismail, C. L. Behrendt, M. Wight-Carter, L. V. Hooper, Z. J. Chen, Mitochondrial antiviral signaling protein (MAVS) monitors commensal bacteria and induces an immune response that prevents experimental colitis. Proceedings of the National Academy of Sciences of the United States of America 108, 17390-17395 (2011).
19. J. M. Breuss, J. Gallo, H. M. DeLisser, I. V. Klimanskaya, H. G. Folkesson, J. F. Pittet, S. L. Nishimura, K. Aldape, D. V. Landers, W. Carpenter, et al., Expression of the beta 6 integrin subunit in development, neoplasia and tissue repair suggests a role in epithelial remodeling. J Cell Sci 108 (Pt 6), 2241-2251 (1995).
20. J. M. Breuss, N. Gillett, L. Lu, D. Sheppard, R. Pytela, Restricted distribution of integrin beta 6 mRNA in primate epithelial tissues. J Histochem Cytochem 41, 1521-1527 (1993).
21. S. Vaishnava, M. Yamamoto, K. M. Severson, K. A. Ruhn, X. Yu, O. Koren, R. Ley, E. K. Wakeland, L. V. Hooper, The antibacterial lectin Reglllgamma promotes the spatial segregation of microbiota and host in the intestine. Science 334, 255-258 (2011).
22. H. Poeck, R. Besch, C. Maihoefer, M. Renn, D. Tormo, S. S. Morskaya, S. Kirschnek, E. Gaffal, J. Landsberg, J. Hellmuth, A. Schmidt, D. Anz, M. Bscheider, T. Schwerd, C. Berking, C. Bourquin, U. Kalinke, E. Kremmer, H. Kato, S. Akira, R. Meyers, G. Hacker, M. Neuenhahn, D. Busch, J. Ruland, S. Rothenfusser, M. Prinz, V. Hornung, S. Endres, T. Tuting, G. Hartmann, 5′-Triphosphate-siRNA: turning gene silencing and Rig-I activation against melanoma. Nature medicine 14, 1256-1263 (2008).
23. J. A. Dudakov, A. M. Hanash, M. R. van den Brink, Interleukin-22: immunobiology and pathology. Annual review of immunology 33, 747-785 (2015).
24. J. A. Dudakov, A. M. Hanash, R. R. Jenq, L. F. Young, A. Ghosh, N. V. Singer, M. L. West, O. M. Smith, A. M. Holland, J. J. Tsai, R. L. Boyd, M. R. van den Brink, Interleukin-22 drives endogenous thymic regeneration in mice. Science 336, 91-95 (2012).
25. A. Dann, H. Poeck, A. L. Croxford, S. Gaupp, K. Kierdorf, M. Knust, D. Pfeifer, C. Maihoefer, S. Endres, U. Kalinke, S. G. Meuth, H. Wiendl, K. P. Knobeloch, S. Akira, A. Waisman, G. Hartmann, M. Prinz, Cytosolic RIG-I-like helicases act as negative regulators of sterile inflammation in the CNS. Nature neuroscience 15, 98-106 (2012).
26. T. Sato, R. G. Vries, H. J. Snippert, M. van de Wetering, N. Barker, D. E. Stange, J. H. van Es, A. Abo, P. Kujala, P. J. Peters, H. Clevers, Single Lgr5 stem cells build crypt-villus structures in vitro without a mesenchymal niche. Nature 459, 262-265 (2009).
27. B. K. Koo, H. Clevers, Stem cells marked by the R-spondin receptor LGR5. Gastroenterology 147, 289-302 (2014).
28. K. Yasuda, P. Yu, C. J. Kirschning, B. Schlatter, F. Schmitz, A. Heit, S. Bauer, H. Hochrein, H. Wagner, Endosomal translocation of vertebrate DNA activates dendritic cells via TLR9-dependent and -independent pathways. Journal of immunology 174, 6129-6136 (2005).
29. H. Ishikawa, Z. Ma, G. N. Barber, STING regulates intracellular DNA-mediated, type I interferon-dependent innate immunity. Nature 461, 788-792 (2009).
30. J. Ahn, D. Gutman, S. Saijo, G. N. Barber, STING manifests self DNA-dependent inflammatory disease. Proceedings of the National Academy of Sciences of the United States of America 109, 19386-19391 (2012).
31. K. Vidal, I. Grosjean, J. P. evillard, C. Gespach, D. Kaiserlian, Immortalization of mouse intestinal epithelial cells by the SV40-large T gene. Phenotypic and immune characterization of the MODE-K cell line. Journal of immunological methods 166, 63-73 (1993).
32. R. J. Robb, E. Kreijveld, R. D. Kuns, Y. A. Wilson, S. D. Olver, A. L. Don, N. C. Raffelt, N. A. De Weerd, K. E. Lineburg, A. Varelias, K. A. Markey, M. Koyama, A. D. Clouston, P. J. Hertzog, K. P. Macdonald, G. R. Hill, Type I-IFNs control GVHD and GVL responses after transplantation. Blood 118, 3399-3409 (2011).
33. M. Tschurtschenthaler, J. Wang, C. Fricke, T. M. Fritz, L. Niederreiter, T. E. Adolph, E. Sarcevic, S. Kunzel, F. A. Offner, U. Kalinke, J. F. Baines, H. Tilg, A. Kaser, Type I interferon signalling in the intestinal epithelium affects Paneth cells, microbial ecology and epithelial regeneration. Gut 63, 1921-1931 (2014).
34. Y. Wang, H. X. Zhang, Y. P. Sun, Z. X. Liu, X. S. Liu, L. Wang, S. Y. Lu, H. Kong, Q. L. Liu, X. H. Li, Z. Y. Lu, S. J. Chen, Z. Chen, S. S. Bao, W. Dai, Z. G. Wang, Rig-I−/−mice develop colitis associated with downregulation of G alpha i2. Cell research 17, 858-868 (2007).
35. A. M. Hanash, J. A. Dudakov, G. Hua, M. H. O'Connor, L. F. Young, N. V. Singer, M. L. West, R. R. Jenq, A. M. Holland, L. W. Kappel, A. Ghosh, J. J. Tsai, U. K. Rao, N. L. Yim, 0. M. Smith, E. Velardi, E. B. Hawryluk, G. F. Murphy, C. Liu, L. A. Fouser, R. Kolesnick, B. R. Blazar, M. R. van den Brink, Interleukin-22 protects intestinal stem cells from immune-mediated tissue damage and regulates sensitivity to graft versus host disease. Immunity 37, 339-350 (2012).
36. L. Pellegrinet, V. Rodilla, Z. Liu, S. Chen, U. Koch, L. Espinosa, K. H. Kaestner, R. Kopan, J. Lewis, F. Radtke, DII1- and dII4-mediated notch signaling are required for homeostasis of intestinal stem cells. Gastroenterology 140, 1230-1240 e1231-1237 (2011).
37. R. L. Gallo, L. V. Hooper, Epithelial antimicrobial defence of the skin and intestine. Nature reviews. Immunology 12, 503-516 (2012).
38. J. Munoz, D. E. Stange, A. G. Schepers, M. van de Wetering, B. K. Koo, S. Itzkovitz, R. Volckmann, K. S. Kung, J. Koster, S. Radulescu, K. Myant, R. Versteeg, O. J. Sansom, J. H. van Es, N. Barker, A. van Oudenaarden, S. Mohammed, A. J. Heck, H. Clevers, The Lgr5 intestinal stem cell signature: robust expression of proposed quiescent ‘+4’ cell markers. The EMBO journal 31, 3079-3091 (2012).
39. K. S. Yan, L. A. Chia, X. Li, A. Ootani, J. Su, J. Y. Lee, N. Su, Y. Luo, S. C. Heilshorn, M. R. Amieva, E. Sangiorgi, M. R. Capecchi, C. J. Kuo, The intestinal stem cell markers Bmi1 and Lgr5 identify two functionally distinct populations. Proceedings of the National Academy of Sciences of the United States of America 109, 466-471 (2012).
40. S. C. Nalle, J. R. Turner, Intestinal barrier loss as a critical pathogenic link between inflammatory bowel disease and graft-versus-host disease. Mucosal Immunol 8, 720-730 (2015).
41. B. Funke, F. Lasitschka, W. Roth, R. Penzel, S. Meuer, M. Saile, N. Gretz, B. Sido, P. Schirmacher, F. Autschbach, Selective downregulation of retinoic acid-inducible gene I within the intestinal epithelial compartment in Crohn's disease. Inflammatory bowel diseases 17, 1943-1954 (2011).
42. D. Jankovic, J. Ganesan, M. Bscheider, N. Stickel, F. C. Weber, G. Guarda, M. Follo, D. Pfeifer, A. Tardivel, K. Ludigs, A. Bouazzaoui, K. Keri, J. C. Fischer, T. Haas, A. Schmitt-Graff, A. Manoharan, L. Muller, J. Finke, S. F. Martin, O. Gorka, C. Peschel, J. Ruland, M. Idzko, J. Duyster, E. Holler, L. E. French, H. Poeck, E. Contassot, R. Zeiser, The NIrp3 inflammasome regulates acute graft-versus-host disease. The Journal of experimental medicine 210, 1899-1910 (2013).
43. O. Alpdogan, S. J. Muriglan, J. M. Eng, L. M. Willis, A. S. Greenberg, B. J. Kappel, M. R. van den Brink, IL-7 enhances peripheral T cell reconstitution after allogeneic hematopoietic stem cell transplantation. The Journal of clinical investigation 112, 1095-1107 (2003).
44. K. Neumann, M. Castineiras-Vilarino, U. Hockendorf, N. Hannesschlager, S. Lemeer, D. Kupka, S. Meyermann, M. Lech, H. J. Anders, B. Kuster, D. H. Busch, A. Gewies, R. Naumann, O. Gross, J. Ruland, Clec12a is an inhibitory receptor for uric acid crystals that regulates inflammation in response to cell death. Immunity 40, 389-399 (2014).
45. T. D. Kim, T. H. Terwey, J. L. Zakrzewski, D. Suh, A. A. Kochman, M. E. Chen, C. G. King, C. Borsotti, J. Grubin, O. M. Smith, G. Heller, C. Liu, G. F. Murphy, O. Alpdogan, M. R. van den Brink, Organ-derived dendritic cells have differential effects on alloreactive T cells. Blood 111, 2929-2940 (2008).

1. A method, administering to a subject in need thereof a therapeutically effective amount of a RIG-I agonist, a cGAS/STING agonist or a combination thereof, wherein the administering is for inhibiting treatment-associated inflammation and graft versus host disease (GVHD), for inhibiting acute intestinal injury during allogeneic hemopoietic stem cell transplantation (allo-HSCT), for inhibiting GVHD following allo-HSCT, to enhance intestinal regeneration in vivo following allo-HSCT, or any combination of two or more of the foregoing.

2. The method of claim 1, wherein the administering is for inhibiting acute intestinal injury during allogeneic hemopoietic stem cell transplantation (allo-HSCT).

3. The method of claim 1, wherein the administration is for inhibiting GVHD following allo-HSCT.

4. A method to promote growth of intestinal organoids in vitro comprising contacting said intestinal organoids with a RIG-I agonist.

5. The method of claim 1, wherein the administration is to enhance intestinal regeneration in vivo following allo-HSCT.

6. The method of claim 1, wherein the RIG-I agonist is selected from 3pRNA; interferon stimulatory DNA (ISD); in vitro transcribed 3pRNA; small endogenous non-coding RNAs (sncRNAs, U1/U2); double stranded RNA such as Poly-ICLC (Hiltonol), MCT-465 (Multicell Technologies), ImOl-100 (Rigontec), and small molecule, Kineta KIN1148, SB-9200 (Spring Bank Pharmaceuticals).

7. The method of claim 1, wherein the cGAS/STING agonist is selected from Interferon stimulatory DNA (ISD); ADU-S100 (Aduro and Novartis): cyclical dinucleotides, 2′,3′ CDNs; alimogene laherparepvec (T-Vec), herpes simplex virus-1 (HSV-1); 5,6-dimethyllxanthenone-4-acetic acid (DMXAA); and Dispiro diketopiperzine (DSDP).

8. The method of claim 1, wherein agonist is administered prior to allo-HSCT.

9. The method of claim 1, wherein agonist is administered from 72 hours prior to transplantation until immediately prior to transplantation.

10. The method of claim 1, wherein agonist is administered from 48 hours prior to transplantation until immediately prior to transplantation.

11. The method of claim 1, wherein agonist is administered from 24 hours prior to transplantation until immediately prior to transplantation.

12. The method of claim 1, wherein agonist is administered from 12 hours prior to transplantation until immediately prior to transplantation.

13. A use of the RIG-I agonist of claim 16, wherein the use is to inhibit intestinal tissue damage induced by radiation or chemotherapeutic conditioning for allo-HSCT.

14. A use of the RIG-I agonist of claim 16, wherein the use is to inhibit GVHD following allo-HSCT.

15. The use of claim 14, wherein GVHD results from pre-transplant conditioning for allo-HSCT.

16. A RIG-I agonist for use in reducing GVHD following allo-HSCT, inhibiting intestinal tissue damage induced by radiation or chemotherapeutic conditioning for allo-HSCT, or both.

17. The method of claim 1, wherein the administering is for inhibiting treatment-associated inflammation and graft versus host disease (GVHD).

18. The method of claim 6, wherein agonist is administered from 48 hours prior to transplantation until immediately prior to transplantation.

19. The method of claim 6, wherein agonist is administered from 24 hours prior to transplantation until immediately prior to transplantation.

20. The method of any claim 6, wherein agonist is administered from 12 hours prior to transplantation until immediately prior to transplantation.
